(19) United States
(12) Patent Application Publication
(10) Pub. No.: US 2017/0065638 A1
(43) Pub. Date: Mar. 9, 2017

(54) REGENERATIVE CELL THERAPY FOR CENTRAL NERVOUS SYSTEM (CNS) DISORDERS AND PTSD

(71) Applicant: Cytori Therapeutics, Inc., San Diego, CA (US)

(72) Inventor: John K. Fraser, San Diego, CA (US)

(21) Appl. No.: 15/117,665

(22) PCT Filed: Feb. 9, 2015
(86) PCT No.: PCT/US15/15028

§ 371 Date: Aug. 9, 2016

Related U.S. Application Data
(60) Provisional application No. 61/938,052, filed on Feb. 10, 2014.
(60) Provisional application No. 61/938,040, filed on Feb. 10, 2014.

(51) Int. Cl. A61K 35/28

(52) U.S. Cl. CPC A61K 35/28

(57) ABSTRACT

Provided herein are compositions comprising regenerative cells (e.g., from adipose tissue), for therapy for central nervous system (CNS) disorders, including disorders characterized by excitotoxicity, neuroinflammation, neurodegeneration, and compromise of the blood brain barrier.

### BACKGROUND

Central nervous system (CNS) disorders affect the brain and spinal cord and account for more hospitalizations, more long-term care, and more chronic suffering than nearly all other disorders combined (JAMA, 2001). At present, there are no known cures for CNS diseases and disorders such as Alzheimer's disease (AD), Parkinson's disease (PD), Huntington's Disease (HD), traumatic brain injury (TBI), amyotrophic lateral sclerosis (ALS), multiple sclerosis (MS), epilepsy, dementia, and the like. Indeed, in most instances, available treatments for CNS diseases offer relatively small symptomatic benefit but remain palliative in nature. As such, the need for therapeutics for use in the treatment, prevention, and amelioration of CNS disorders is manifest.

Post-traumatic stress disorder (PTSD) is an anxiety-type disorder that is triggered when an individual experiences or sees a traumatic event, often involving the threat of injury or death. PTSD frequently occurs in veterans and survivors of physical and sexual assault, abuse, accidents, disasters, and many other serious events. Statistics regarding PTSD indicate that approximately 7%-8% of people in the United States will likely develop PTSD in their lifetime, with the lifetime occurrence (prevalence) in combat veterans and rape victims ranging from 10% to as high as 30%.

PTSD alters the body's response to stress and otherwise has pronounced effects on the psychological and physical health of the individual. Specifically, victims of PTSD experience three main types of symptoms: (1) re-experiencing the traumatic events; (2) numbing avoiding reminders of the trauma; and (3) increased anxiety and emotional arousal, or hyperarousal. Re-experiencing can include intrusive memories, nightmares, flashbacks, exaggerated reactions to reminders of the event, and re-experiencing (including re-experiencing physical symptoms when the body ‘remembers’). Numbing/avoidance is associated with a loss of interest in life and other people, hopelessness, isolation, avoidance of thoughts and feelings associated with the traumatic event, feeling detached and estranged from others, withdrawal, depression, and emotional anesthesia. Preoccupation with avoiding trauma or feelings and thoughts related to trauma can become a central focus of the survivor's life. Hyperarousal is associated with difficulty sleeping and concentrating, being easily startled, irritability, anger, agitation, panic, and hypervigilance (being hyper-alert to danger). The symptoms of post-traumatic stress disorder (PTSD) can arise suddenly, gradually, or come and go over time, and sometimes appear for no apparent cause. At other times, the symptoms are triggered by something that reminds the victim of the original traumatic event, such as a noise, an image, certain words, or a smell. Accordingly, PTSD gravely affects the victims' life and well-being.

Current treatments for PTSD often involve cognitive therapy, in which individuals “relive” the traumatic event in an effort to desensitize them. Unfortunately, these treatments are not entirely satisfactory. A similar kind of therapy is eye movement desensitization and reprocessing (EMDR) that is used for PTSD. Selective serotonin reuptake inhibitors (SSRI), which are also used for depression, are also commonly prescribed for individuals suffering from PTSD. As these therapies are unsatisfactory, there is a need for new compositions and methods of treatment for individuals with PSTD.

### SUMMARY

Provided herein are compositions and methods related to the use of regenerative cells (and optionally secretions therefrom), in the treatment of conditions and disorders of the central nervous system, e.g., that are associated with excitotoxicity. These embodiments therefore include, for example compositions and methods useful for treating, preventing, ameliorating, etc., conditions such as severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury and amyotrophic lateral sclerosis.

In a first aspect, provided is a method for reducing vulnerability to secondary brain insult in a subject in need thereof, that includes identifying a subject that has suffered one or more mild or severe traumatic brain injuries and administering a composition comprising regenerative cells (e.g., adipose-derived regenerative cells, bone marrow-derived regenerative cells, placental-derived regenerative cells, and the like), to said subject. Also provided are uses of regenerative cells for reducing vulnerability to secondary brain insult in a subject that has suffered one or more mild or severe traumatic brain injuries. In some embodiments, method of claim 1, wherein the composition comprising regenerative cells is administered to the subject within 24 hours of said one or more mild or severe traumatic brain injuries. In some embodiments, the method also includes measurement of the cerebral metabolism of the subject. For example, in specific embodiments, the subject has a below average level of cerebral metabolism at the time of administration of the composition comprising regenerative cells.

In another aspect, provided is a method of modulating microglial activation in a subject in need thereof, including identifying a subject in need of modulation of microglial activation and administering a composition comprising regenerative cells to the subject. Also provided are uses of regenerative cells (e.g., adipose-derived regenerative cells, bone marrow-derived regenerative cells, placental-derived regenerative cells, and the like), for modulating microglial activation in a subject in need thereof. For example, the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury and amyotrophic lateral sclerosis. In some embodiments, the modulation of microglial activation comprises inhibition of nitric oxide (NO) production by microglia. In some embodiments, the modulation of microglial activation comprises increasing the ratio of M2 to M1 microglial cells. In some embodiments, the modulation of microglial activation comprises recuing the concentration of reactive oxygen species (ROS) and/or reactive nitrogen species (RNS) in the central nervous system. In some embodiments, the modulation of microglial activation comprises decreasing the expression and/or secretion of a factor selected from the group consisting of: NO, TNFα, IL-1β, IL-12, IL-18 and a matrix metalloproteinase (MMP), e.g., MMP-9. In some embodiments, the modulation of microglial activation comprises increasing the expression and/or secretion of a factor selected from the group consisting of: TGF-β, IL-10, and IL-15. In some embodiments, the modulation of microglial activation comprises decreasing the number of calcium permeable AMPA receptors on the microglia.

In another aspect, provided is a method of preventing synaptic and/or dendritic loss in a subject in need thereof that includes identifying a subject in need of prevention of synaptic and/or dendritic loss, and administering a composition comprising regenerative cells to the subject. Also provided are compositions comprising regenerative cells, (e.g., adipose-derived regenerative cells, bone marrow-derived regenerative cells, placental-derived regenerative cells, and the like), for preventing synaptic and/or dendritic loss in a subject in need thereof. For example, the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury and amyotrophic lateral sclerosis.

In another aspect, provided herein is method of increasing the loco-regional concentration of M2 macrophages in the CNS that includes identifying a subject in need of a loco-regional increase in M2 macrophages and/or microglia, and administering a composition comprising regenerative cells (e.g., adipose-derived regenerative cells, bone marrow-derived regenerative cells, placental-derived regenerative cells, and the like), to the subject. Also provided are compositions comprising regenerative cells for increasing the loco-regional concentration of M2 macrophages in the CNS in subjects in need thereof. For example, the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury and amyotrophic lateral sclerosis.

In yet another aspect, provided is a method of preventing neuronal apoptosis in a subject in need thereof, including identifying a subject in need of prevention of neuronal apoptosis, and administering a composition comprising regenerative cells (e.g., adipose-derived regenerative cells, bone marrow-derived regenerative cells, placental-derived regenerative cells, and the like), to the subject. Also provided are compositions comprising regenerative cells to a subject in need of prevention of neuronal apoptosis. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

In another aspect, provided is a method of enhancing cerebral blood flow in a subject in need thereof, including identifying a subject in need of restoration of cerebral blood flow, and administering a composition comprising regenerative cells (e.g., adipose-derived regenerative cells, bone marrow-derived regenerative cells, placental-derived regenerative cells, and the like), to the subject. Also are provided uses of compositions comprising regenerative cells for enhancing cerebral blood flow in subjects in need thereof. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

Yet another aspects provides a method of improving or repairing blood brain barrier integrity in a subject in need thereof, including identifying a subject in need of improvement in blood brain barrier integrity; and administering a composition comprising regenerative cells (e.g., adipose-derived regenerative cells, bone marrow-derived regenerative cells, placental-derived regenerative cells, and the like), to the subject. Also provided are uses of compositions comprising regenerative cells for improving or repairing blood brain barrier integrity in subjects in need thereof. Also are provided uses of compositions comprising regenerative cells for enhancing cerebral blood flow in subjects in need thereof. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

Yet other aspects provide a method of increasing the pericyte:endothelial ratio in the blood brain barrier of subject in need thereof, including identifying a subject in need an increase in pericyte:endothelial cell ratio in the blood brain barrier; and administering a composition comprising regenerative cells (e.g., adipose-derived regenerative cells, bone marrow-derived regenerative cells, placental-derived regenerative cells, and the like), to the subject. Also provided are uses of compositions comprising regenerative cells for increasing the pericyte:endothelial ration in the blood brain barrier in a subject in need thereof. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

In another aspect, provided is a method of reducing leukocyte infiltration into the central nervous system of a subject in need thereof, including identifying a subject in need of reduced leukocyte number in the central nervous system; and administering a composition comprising regenerative cells (e.g., adipose-derived regenerative cells, bone marrow-derived regenerative cells, placental-derived regenerative cells, and the like), to the subject. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

Also provided are methods of increasing cerebral metabolism in a subject in need thereof, including identifying a subject in need of increased cerebral metabolism (e.g., a subject with decreased cerebral metabolism); and administering a composition comprising regenerative cells (e.g., adipose-derived regenerative cells, bone marrow-derived regenerative cells, placental-derived regenerative cells, and the like), to the subject. Also provided are uses of compositions comprising regenerative cells for increasing cerebral metabolism in subjects in need thereof. For example the subject may have one or more of the following conditions: severe traumatic brain injury, mild traumatic brain injury, repeated traumatic brain injury, epilepsy, dementia, PTSD, multiple sclerosis, Alzheimer's disease, Huntington's disease, Parkinson's Disease, spinal cord injury, and amyotrophic lateral sclerosis.

In the embodiments described above, the regenerative cells are adipose-derived regenerative cells. In some embodiments, the regenerative cells comprise at least 0.1% adipose-derived stem cells. In some embodiments, the regenerative cells comprise adipose-derived stem cells and precursor cells. In some embodiments, the regenerative cells comprise one or more adipose-derived cells selected from the group consisting of stem cells, precursor cells, progenitor cells, endothelial cells, and leukocytes.

### BRIEF DESCRIPTION OF THE DRAWINGS

FIG. 1 is a schematic of the series of events that characterize neuroexcitotoxicity.

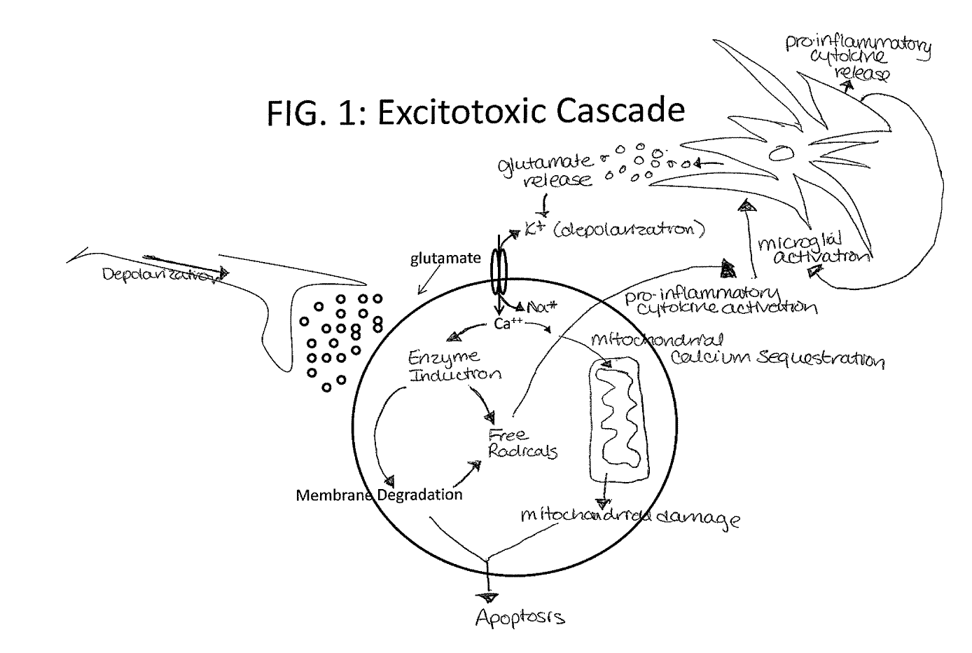

FIG. 2 is a schematic depicting signals received and released from microglial cells.

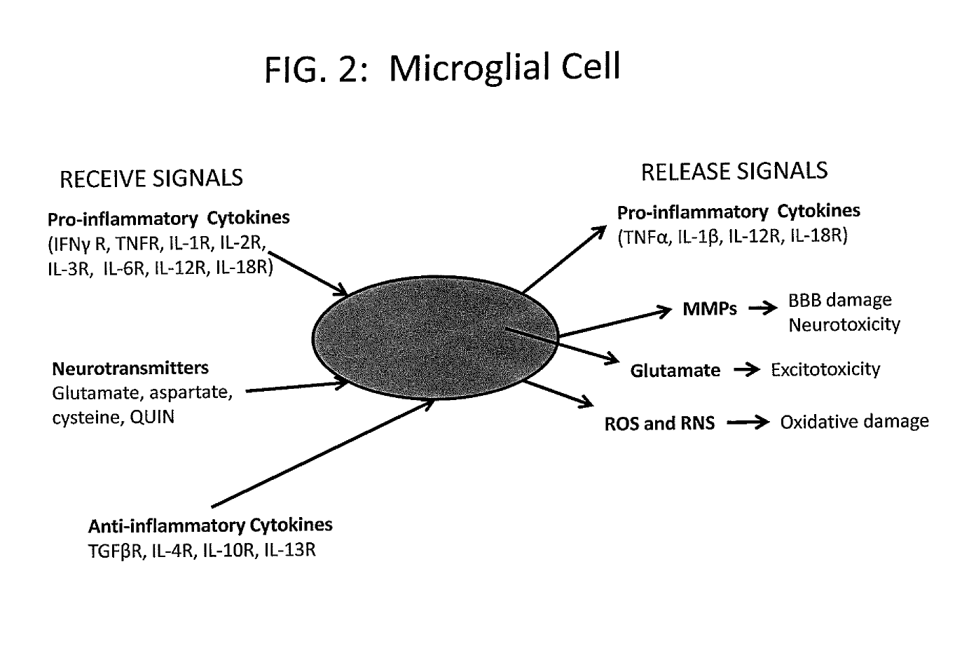

### DETAILED DESCRIPTION

Most, if not all, neuropathologies are to a various extent associated with glial activation. See, Glial Physiology and Pathophysiology, Verkhratsky, ed. © 2013 John Wiley & Sons, Hoboken, N.J. Many neuropathies share a common constellation of features including for example, neuroexcitotoxicity and neuroinflammation, and for those neuropathies relating to the brain in particular, decreased cerebral metabolism, decreased cerebral blood flow, and disrupted permeability of the blood brain barrier (BBB), as described in further detail below. Each of these features manifests in traumatic brain injury, PTSD, hypoxia, multiple sclerosis, Alzheimer's disease, epilepsy, Parkinson's disease, Huntington's Disease, epilepsy, fibromyalgia, amyotrophic lateral sclerosis (ALS), and spinal cord injury (“SCI”). Cerebral palsy can arise as a result of trauma to the infant's brain and/or severe oxygen deficiency. Accordingly, the embodiments disclosed herein are useful for the treatment, prevention, or amelioration of cerebral palsy inasmuch as this disease shares patho-physiological features with the various neuropathologies discussed herein. The embodiments disclosed herein are based, in part, on the ability of adipose-derived cells (e.g., adipose-derived regenerative cells, such as concentrated populations of adipose-derived cells comprising stem cells) to modulate, e.g., inhibit or reduce inflammation and/or neuroexcitotoxcitiy and associated glial activation in the CNS, improve cerebral metabolism and blood flow, and improve or restore BBB integrity, and recruit cells useful in CNS regeneration (e.g., neural stem cells and the like) to the CNS, and in particular to sites of injury and/or apoptosis. Accordingly, provided herein are methods and compositions that include regenerative cells, e.g., adipose-derived regenerative cells (e.g., stem cells, precursor cells, and combinations thereof), useful in the treatment, amelioration, or prevention of traumatic brain injury, hypoxia, multiple sclerosis, Alzheimer's disease, Parkinson's disease, Huntington's Disease, epilepsy, fibromyalgia and amyotrophic lateral sclerosis (ALS), cerebral palsy, spinal cord injury, and other CNS disorders that exhibit one or more of the shared physiological features discussed herein.

### DEFINITIONS

As used herein, the term “about,” when referring to a stated numeric value, indicates a value within plus or minus 10% of the stated numeric value.

As used herein, the term “derived” means isolated from or otherwise purified. For example, adipose derived stem and other regenerative cells are isolated from adipose tissue. The term “derived” does not encompass cells that are extensively cultured (e.g., placed in culture conditions in which the majority of dividing cells undergo 3, 4, 5 or less, cell doublings), from cells isolated directly from a tissue, e.g., adipose tissue, or cells cultured or expanded from primary isolates. Accordingly, “adipose derived cells,” including adipose-derived stem and other regenerative cells and combinations thereof, refers to cells obtained from adipose tissue, wherein the cells are not extensively cultured, e.g., are in their “native” form as separated from the adipose tissue matrix.

As used herein, a cell is “positive” for a particular marker when that marker is detectable. For example, an adipose derived regenerative cell is positive for, e.g., CD73 because CD73 is detectable on an adipose derived stem or regenerative cell in an amount detectably greater than background (in comparison to, e.g., an isotype control or an experimental negative control for any given assay). A cell is also positive for a marker when that marker can be used to distinguish the cell from at least one other cell type, or can be used to select or isolate the cell when present or expressed by the cell.

In some contexts, the term “adipose tissue” refers to a tissue containing multiple cell types including adipocytes and vascular cells. Adipose tissue includes multiple regenerative cell types, including adult stem cells (ASCs), endothelial progenitor and precursor cells, pericytes and the like. Accordingly, adipose tissue refers to fat, including the connective tissue that stores the fat.

In some contexts, the term “unit of adipose tissue” refers to a discrete or measurable amount of adipose tissue. A unit of adipose tissue may be measured by determining the weight and/or volume of the unit. In reference to the disclosure herein, a unit of adipose tissue may refer to the entire amount of adipose tissue removed from a subject, or an amount that is less than the entire amount of adipose tissue removed from a subject. Thus, a unit of adipose tissue may be combined with another unit of adipose tissue to form a unit of adipose tissue that has a weight or volume that is the sum of the individual units.

In some contexts, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a subject is a portion of the removed adipose tissue.

As used herein, “regenerative cells” refers to any heterogeneous or homogeneous population of cells obtained using the systems and methods of embodiments disclosed herein which cause or contribute to complete or partial regeneration, restoration, or substitution of structure or function of an organ, tissue, or physiologic unit or system to thereby provide a therapeutic, structural or cosmetic benefit. Examples of regenerative cells include: ASCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes. Accordingly, adipose-derived regenerative cells (“ADRCs”) as used herein refers to any heterogeneous or homogeneous cell population that contains one or more types of adipose-derived regenerative cells including adipose-derived stem cells, endothelial cells (including blood and lymphatic endothelial cells), endothelial precursor cells, endothelial progenitor cells, macropahges, fibroblasts, pericytes, smooth muscle cells, preadipocytes, kertainocytes, unipotent and multipotent progenitor and precursor cells (and their progeny), and lymphocytes. By way of example, the stromal vascular fraction (SVF) of adipose tissue comprises ADCs. In preferred embodiments, adipose-derived stem cells comprise at least 0.1% of the cellular component of adipose-derived cells, e.g., 2-12% of the cellular component. In some embodiments, adipose-derived stem cells comprise 15%, 20%, 30%, 40%, 50%, 60%, 70%, or more, up to 100% of the adipose-derived cells. As explained in further detail below, regenerative cells useful in the embodiments disclosed herein can be derived from various tissue types including, but not limited to bone marrow, adipose tissue, placental tissue, muscle, dental pulp, and the like.

In some contexts, the term “progenitor cell” refers to a cell that is unipotent, bipotent, or multipotent with the ability to differentiate into one or more cell types, which perform one or more specific functions and which have limited or no ability to self-renew. Some of the progenitor cells disclosed herein may be pluripotent.

In some contexts, the term “adipose tissue-derived cells” refers to cells extracted from adipose tissue that has been processed to separate the active cellular component (e.g., the cellular component that does not include adipocytes and/or red blood cells) from the mature adipocytes and connective tissue. Separation may be partial or full. That is, the “adipose tissue-derived cells” may or may not contain some adipocytes and connective tissue and may or may not contain some cells that are present in aggregates or partially disaggregated form (for example, a fragment of blood or lymphatic vessel comprising two or more cells that are connected by extracellular matrix). This fraction is referred to herein as “adipose tissue-derived cells,” “adipose derived cells,” “adipose derived regenerative cells” or “ADC.” Typically, ADC refers to the pellet of cells obtained by washing and separating the cells from the adipose tissue, e.g., the stromal vascular fraction. The pellet is typically obtained by concentrating a suspension of cells released from the connective tissue and adipose tissue matrix. By way of example, the pellet can be obtained by centrifuging a suspension of adipose-derived cells so that the cells aggregate at the bottom of a centrifuge container, e.g., the stromal vascular fraction. In some embodiments, the adipose-derived cell populations described herein include, among other cell types, leukocytes. In some embodiments, the adipose-derived cell populations described herein include, among other regenerative cell types, endothelial cells. In some embodiments, ADCs comprise adipocytes.

Adipose tissue derived cells can release microvesicles and exosomes, e.g., particles between about 40 and 1000 nm which can fuse with cellular membranes of different cell types, thereby transferring their contents into cells with which they fuse. As used herein, the term “adipose-derived micro-particles” (“ADMPs”) refers to adipose-derived microvesicles and exosomes. In some embodiments, the ADMPs comprise nucleic acids, such as mRNAs, microRNAs (“miRNAs”), DNA and the like In some embodiments the ADMPs comprise proteins such as cytokines, growth and trophic factors (e.g., angiogenic and arteriogenic factors, neurotrophic factors and the like), proteases (e.g., neprilysin, anti-fibrotic proteins, and the like), and other proteins. As used herein, the term “neurotrophic factor” refers to any factor that promotes the growth, survival and maintenance of neurons. Exemplary neurotrophic factors include nerve growth factor (NGF), brain-derived neurotrophic factor (BDNF), ciliary neurotrophic factor (CNTF), neutrophin-3 (NT-3), neutrophin-4/5 (NT-4/5), neutrophin-6 (NT-6), glia cell-derived neurotrophic factor (GDNF), axogenesis factor (AF-1) and glia growth factor (GGF2), and the like.

The embodiments disclosed herein relate to methods and compositions for the treatment of various central nervous system disorders in subjects in need thereof, and methods and compositions for the treatment of PTSD in subjects in need thereof. In certain embodiments, the subject may be a mammal. The mammal may be selected from the group consisting of mice, rats, rabbits, guinea pigs, dogs, cats, sheep, goats, cows, horses, primates, such as monkeys, chimpanzees, and apes, and humans. In some embodiments, the subject is a human. The term “subject” can be used interchangeably with the terms “individual” and “patient” herein.

As used herein, the terms “treat” or “treating” or “treatment” refers to any indicia of success in the treatment, amelioration, or prevention of an injury, pathology, condition, or symptom (e.g., motor function, cognitive function, myelenization, or other art-recognized symptoms or characteristics of the disorders disclosed herein), including any objective or subjective parameter such as abatement; remission; diminishing of symptoms or making the symptom, injury, pathology or condition more tolerable to the subject; decreasing the frequency or duration of the symptom or condition; or, in some situations, preventing the onset of the symptom or condition. The treatment, amelioration or prevention of symptoms can be based on any objective or subjective parameter as discussed in connection with the various CNS disorders discussed herein including, e.g., the result of a physical examination/diagnostic test or the like.

As used herein, the term “modulate” means to change, e.g., to increase or decrease. For example, the phrase “modulation of cytokine expression” can refer to an increase (e.g., by more than 5%, 10%, 15%, 20%, 25%, 30%, 50%, 100% or more), or a decrease (e.g., by by more than 5%, 10%, 15%, 20%, 25%, 30%, 50%, 100% or more), of cytokine levels. In another example, the phrase “modulate the immune response” or “immunomodulation” can refer to an alteration of the immune response, e.g., shifting of T cell and/or macrophage subpopulations and the like. Likewise, the phrase “modulate inflammation” can refer to an alternation of the inflammatory status, as evidenced by a change in the concentration of one or more cytokines, an increase or decrease in the relative concentrations of one or more cell types (e.g., M1:M2 macrophages and/or microglia, various T-cell populations and the like).

### Excitotoxicity and Neuroinflammation

Excitotoxicity is a term used to describe neuronal cell death induced by a cascade of events associated with excessive stimulation of certain cells of the CNS by neurotransmitters such as glutamate and similar substances, e.g., aspartate, cysteine, and quinolinic acid (QUIN). Neuronal excitotoxicity is a hallmark of CNS diseases and disorders including, but not limited to, PTSD, traumatic brain injury (including mild, moderate or severe trauma, repeated trauma, and chronic traumatic encephalopathy), hypoxia, spinal cord injury, and neurodegenerative diseases of the central nervous system (CNS) such as Multiple sclerosis, Alzheimer's disease, Amyotrophic lateral sclerosis (ALS), Fibromyalgia, Parkinson's disease, and Huntington's disease. As explained in further detail below, regenerative cells, (e.g., adipose-derived regenerative cells, such as concentrated populations of adipose-derived cells comprising stem cells) and/or cells and/or adipose-derived micro-particles (ADMPs) disclosed herein are useful in dampening, reducing and preventing excitotoxicity and/or one or more downstream effects of neuroexcitotoxicity.

In addition, stress exposure elicits excitotoxicity due to the massive release of the excitatory amino acid, glutamate, in some brain areas, such as the prefrontal cortex. See, e.g., Moghaddam, et al. (1993), J. Neurochem. 60:1650-1657. Stress is also known to induce the release of pro-inflammatory cytokines such as tumor necrosis factor-α (TNF-α) or interleukin 1/3. See, e.g. Madrigal et al, (2002) Neuropsychopharmacology 26:155-163. Stress also activates the nuclear transcription factor-kappa B (NF-κB) pathway in a TNF-α-dependent mechanism. Bierhaus et al., (2003) Proc. Nat. Acad. Sci. USA 100:1920-1925; see also, Madrigal et al., (2002) Neuropsychopharmacology 26:155-163. NF-κB activation elicits the expression and activity of proinflammatory enzymatic sources, such as inducible nitric oxide (NO) synthase (NOS-2), and cyclooxygenase-2 (COX-2), among others. See, Madrigal, et al. (2001) J. Neurochem. 76(2): 532-538; see also, Madigral, et al. (2003), Neuropsychopharmacol. 28:1579-1588. As discussed further below, the result of this sequence of events is the accumulation of oxidative and nitrosative mediators, which can attack membrane phospholipids and cause cell damage in a process known as lipid peroxidation. See, McEwen, et al. (1998) N. Engl. J Med 338:171-179; see, also, Madrigal, et al, (2001) Neuropsychopharmacol. 24(4): 420-429.

The concentration equilibrium of neurotransmitters such as glutamate in the CNS is extremely delicate, and under normal conditions these neurotransmitters are usually found in millimolar amounts extracellularly. Glutamate is the most abundant neurotransmitter in the brain, and is used in 50% of the synapses in the CNS overall and 90% in the cortex. The term “excitotoxicity” was first coined in 1969 to describe a reaction that occurs when neurons are exposed to excess extracellular glutamate, leading to a delayed reaction and ultimately resulting in neuronal death. Olney et al. (1969) Science 166:386-388. Subsequent studies demonstrated that it was the uncontrolled entry of calcium into the neuron through glutamate-receptor controlled calcium channels that caused the excitatory response, and that calcium activated a number of death events by triggering cell death signaling pathways.

Excess glutamate over-activates neural receptors such as NMDA receptors (NMDA R) and AMPA receptors on postsynaptic cells. Normally, AMPA receptors are calcium impermeable, but under conditions of trauma, hypoxia/ischemia, and in neurodegenerative diseases, such as those mentioned herein above, there is a switch to AMPA receptors that are calcium permeable. Over-activation of NMDA and AMPA receptors causes potassium release and neuronal depolarization, and a large calcium influx into the cell, which has numerous deleterious effects. For example, a large calcium influx overloads neuronal mitochondria, resulting in dysfunction of the cells' energy production capabilities, including uncoupling of oxidative phosphorylation within the electron transport chain. This leads to the creation of damaging reactive oxygen species (ROS), lipid peroxidation products (LPPs), and nitric oxide (NO). ROS, LPPs and NO have been shown, in turn, to interfere with glutamate clearance, thus further exacerbating and magnifying the neuronal excitotoxicity, and causing synaptic and dendritic loss. The large calcium influx also activates a number of enzymes, including phospholipases, endonucleases, and proteases such as calpain and matrix metalloproteases (MMPs). These enzymes damage cellular structures including components of the cytoskeleton, membrane, DNA, and extracellular matrix. As a consequence, cell death signaling pathways, e.g., apoptosis, are triggered. These events have also been shown to cause accumulation of altered proteins, e.g., amyloid-β and/or hyperphosphorylated Tau protein implicated in many neuropathologies.

The principal sources of glutamate are microglia and astrocytes and as such, these cells are central to the phenomenon of neuroexcitotoxcitiy. Microglia are the major resident immune cells of the brain and serve as the resident macrophages of the CNS. Microglial cells have various modes of activity, some of which are predominantly reparative and some that are potentially neurodestructive. Specifically, microglia can exist in a “resting” state, a “primed” state, and an “activated” state. The exact phenotype and physiology of each stage of activation is determined by a number of cues, including extra-neuronal molecules (e.g., excitatory amino acids, chemokines, cytokines and the like) and environmental conditions.

Microglia exhibit varying morphology that correlates with their modes of activity. Resting microglia exhibit a ramified morphology and express low levels of many cell surface immune molecules, including for example CD45, Fc receptors, and MHC class II molecules. See, Perry, et al. (2013) Semin. Immunopathol. 35:601-612. “Primed microglia” have a reduced threshold of activation. Priming of microglia arises as a result of ageing, brain trauma, and the like, and is known to exacerbate disease. Primed microglia have a morphological appearance similar to activated microglia discussed below, but do not express inflammatory cytokines. TNFα, IFN-γ, and complement are all implicated in microglial priming. See, Fenn, et al. (2013) Biol. Psychiatry, doi: 10.1016/j.biopscyh.2013.10.014; Norden, et al. (2013) Neuropathol. Appl. Neurobiol. 39(1):19-34; Ramaglia, et al. (2012) Proc. Nat. Acad. Sci. USA 109(3):965-970. “Activated microglia” exhibit an amoeboid morphology, increased phagocytic ability and enhanced migratory capacity within the brain, along with increased expression of cell surface glycoproteins, including CD45 and MHC class II molecules. Activated microglia release glutamate and other excitotoxins, and enhance permeability of the BBB through expression of TNF-α. See, Prat, et al. (2001) Glia 36: 145-55.

Activated microglia have been characterized into two main phenotypic classes, e.g., M1 and M2, resembling the M1 and M2-like phenotypes of peripheral macrophages. The “activated” M1 microglial phenotype is characterized by strong antigen-presenting abilities, proinflammatory cytokine production (e.g., IL-1, IL-6 and TNFα) and the production of toxic intermediates such as nitric oxide and reactive oxygen species. Activated M1 microglia also exhibit increased expression of receptors for pro-inflammatory cytokines, including Il-1β. See, Appel, et al. (2009) Trends Inmmunol. 31(1):7-17. Activation of M1 microglia occurs from exposure to pro-inflammatory cytokines such as TNFα, IL-1β. As these cytokines themselves activate microglia the excitotoxicity pattern is worsened via an autocrine mechanism.

“Alternatively activated” M2 microglia and macrophages are involved in tissue repair and wound-healing processes and are characterized by high levels of arginase expression and arise in response to cytokines such as IL-4, IL-10 and IL-13. Alternatively activated microglia are known to have neuroprotective functions, including promotion of angiogenesis and matrix remodeling, suppression destructive immunity, provision of trophic support by physically ensheathing neurons under damaging or regenerating conditions, and phagocytosis of debris and clearance of apoptotic cells. See, Perry, et al. (2010) Nat. Rev. Neurol. 6:193-201. Various cytokines are known to polarize macrophages to an M1 or M2-like phenotype. For example, classical activation and M1 polarization arise from IFN-γ and Toll-like receptor-4 (“TLR-4”) signalling. M2 polarization can occur in response to IL-4, IL-10 and IL-13. See, Cao, et al. (2013) Neurosci. Bull. 29(2): 189-198. Exposure of microglia to TGF-β causes the microglia to secrete large amounts of IL-10, thereby implicating TGF-β as a signal to polarize microglia to the alternatively activated M2 state. See, Zhao, et al. (2012) J. Neuroinflamm. 9:210. Accordingly, neuroexcitotoxicity can give rise to neuroinflammation, i.e., an inflammatory response in the CNS that damages components of the nervous system.

Inflammation is found to be a component of many neurodegenerative diseases and adds to the pathogenesis of the neurodegeneration (Minagar, et al. (2002) J. Neurological Sci. 202:13-23; Antel and Owens (1999) J. Neuroimmunol. 100: 181-189; Elliott (2001) Mol. Brain. Res. 95:172-178; Nakamura (2002) Biol. Pharm. Bull. 25:945-953; Whitton P S. (2007) Br J Pharmacol. 150:963-76). Injury to myelin is mediated by an inflammatory response (Ruffini et. al. (2004) Am. J. Pathol. 164:1519-1522).

The regenerative cells, including adipose-derived cells (e.g., adipose-derived regenerative cells, such as concentrated populations of adipose-derived cells comprising stem cells) and/or adipose-derived micro-particles disclosed herein can function to modulate inflammatory events in the CNS. For example, the regenerative cells (e.g., adipose-derived, bone marrow-derived, placental-derived, etc.), can dampen the production and release of inflammatory cytokines, such as TNF-α, IL-1, and IL-6, e.g., in microglia, astrocytes, perivascular macrophages, peripheral macrophages (e.g., that may or may not ultimately infiltrate the CNS), or the like. Regenerative cells, e.g., adipose-derived cells (e.g., adipose-derived regenerative cells, such as concentrated populations of adipose-derived cells comprising stem cells) disclosed herein can function to produce and secrete, or deliver, IL-1 receptor antagonists. Regenerative cells, e.g., adipose-derived cells (e.g., adipose-derived regenerative cells, such as concentrated populations of adipose-derived cells comprising stem cells), and/or adipose-derived micro-particles disclosed herein can function to inhibit complement-mediated activation of microglia.

Accordingly, in some embodiments, the regenerative cells, e.g., adipose-derived cells (e.g., adipose-derived regenerative cells, such as concentrated populations of adipose-derived cells comprising stem cells) and/or adipose-derived micro-particles are used to modulate inflammation in the CNS, for example by reducing microglial activation in a subject in need thereof, e.g., a subject that has been diagnosed with or who has been identified as being at risk of PTSD, traumatic brain injury (including mild, moderate, or severe trauma, repeated trauma, and chronic traumatic encephalopathy), multiple sclerosis, Alzheimer's disease, Amyotrophic lateral sclerosis (“ALS”), Fibromyalgia, Parkinson's disease, and Huntington's disease, epilepsy, spinal cord injury, cerebral palsy or the like. In some embodiments, microglial activation is reduced by more than 5%, more than 10%, more than 20%, more than 25%, more than 30%, more than 35%, more than 40%, more than 45%, more than 50%, more than 60%, more than 70%, more than 80%, more than 90%, or more, in subjects receiving treatment with adipose-derived regenerative cells as described herein, compared to control subjects (i.e., untreated subjects that have, or are at risk of developing PTSD, Multiple sclerosis, Alzheimer's disease, Amyotrophic lateral sclerosis (ALS), Fibromyalgia, Parkinson's disease, Huntington's disease, epilepsy, spinal cord injury, cerebral palsy or the like). The presence and/or abundance of activated microglia in the CNS can be readily measured using art-accepted techniques, e.g., using positron emission tomography scanning (PET scanning). [¹¹C](R)PK11195-PET is a marker of activated microglia, used in PET scanning. Edison, et al. (2008), Neurobiol. Dis. 32(3): 412-419. Microglial activation is also characterized by overexpression of mitochondrial 18 kDa Translocator Protein (TSPO). TSPO expression can be quantified in-vivo using the positron emission tomography (PET) radioligand [18F]-FEPPA. Suridjan, et al. (2014) NeuroImage 84:868-875. Accordingly, some embodiments include the steps of identifying a subject in need of a reduction in microglial activation, and administering a composition comprising regenerative cells, e.g., adipose-derived cells (e.g., adipose-derived regenerative cells, such as concentrated populations of adipose-derived cells comprising stem cells), as described elsewhere herein to the subject. In some embodiments, the method can include the further step of measuring microglial activation in the subject. Accordingly, some embodiments include the steps of identifying a subject that has or that is at risk of developing PTSD, and administering a composition comprising regenerative cells, e.g., adipose-derived cells (e.g., adipose-derived regenerative cells such as concentrated populations of adipose-derived cells comprising stem cells) and/or adipose-derived micro-particles as described elsewhere herein to the subject. In some embodiments, the method can include the further step of measuring microglial activation in the subject. Some embodiments include the steps of identifying a subject that has, or that is a risk of developing, Multiple sclerosis, Alzheimer's disease, Amyotrophic lateral sclerosis (ALS), Fibromyalgia, Parkinson's disease, Huntington's disease, epilepsy, spinal cord injury, cerebral palsy or the like.

The regenerative cells, e.g., bone marrow-derived cells, placental derived cells, adipose-derived cells (e.g., adipose-derived regenerative cells, such as concentrated populations of adipose-derived cells comprising stem cells), etc., disclosed herein shift macrophage differentiation from a pro-inflammatory M1 type to an anti-inflammatory M2 type, thereby treating, inhibiting or ameliorating PTSD, and/or the CNS disorders discussed herein. For example, in some embodiments, the regenerative cells, e.g., adipose-derived cells disclosed herein secrete or increase the concentration of TGF-β and/or IL-10, thereby shifting microglia from an M1 to an M2 phenotype. Accordingly, in some embodiments, the regenerative cells, e.g., adipose-derived cells disclosed herein (e.g., concentrated populations of adipose-derived cells comprising stem cells, precursor cells, progenitor cells, and the like) and/or adipose-derived micro-particles, increase the ratio of M2:M1 activated microglial cells in a subject in need thereof, e.g., a subject that has been diagnosed with or who has been identified as being at risk of traumatic brain injury (including severe trauma, moderate trauma, mild trauma, repeated trauma, and chronic traumatic encephalopathy), PTSD, multiple sclerosis, Alzheimer's disease, ALS, fibromyalgia, Parkinson's disease, Huntington's disease, epilepsy, spinal cord injury, cerebral palsy, or the like. Accordingly, some embodiments relate to a method of increasing the ratio of M2:M1 activated microglial cells in a subject in need thereof, including identifying a subject in need of an increase in the ratio of M2:M1 activated microglial cells, and administering a composition comprising regenerative cells, e.g., bone marrow-derived, placental derived, adipose-derived cells (e.g., a concentrated population of adipose-derived regenerative cells comprising stem cells), etc. to said subject as described elsewhere herein. For example, in some embodiments, the regenerative cells, e.g., adipose-derived cells disclosed herein can increase the ratio of M2:M1 cells to greater than 1:1, greater than 1.5:1; greater than 2:1; greater than 2.5:1; greater than 3:1, greater than 3.5:1; greater than 4:1, or the like. The ratio of M2:M1 cells can be readily determined using art-accepted means, including for example, measuring the ratio of CD206/CD86 cell surface markers (e.g., in the blood) as described in Walker et al. (2012) J. Neuroinflamm. 9:228. In some embodiments, the method includes the further step of measuring the ratio of M2:M1 activated microglia in the subject.

Adipose tissue contains M2-like macrophages. Zeyada, et al. (2007) Int. J. Obes. 31:1420-1428. Regenerative cells (e.g., adipose-derived regenerative cells, such as concentrated populations of adipose-derived cells comprising stem cells) and/or adipose-derived microparticles disclosed herein can increase the loco-regional concentration of M2 macrophages in the CNS. An increase in the number of M2 macrophages and microglia can promote CNS repair while limiting secondary inflammatory-mediated injury in the CNS disorders discussed herein. See, e.g., Kigert (2009) J. Neurosci. 29(43):13435-13444; Rawji, et al., (2013) Clin. Devel. Immunol. 2013: Article ID 948976; Chernykh (2010) Cell Ther. Transplant. 2(6):1. Accordingly, in some embodiments, the regenerative cells, e.g., adipose-derived regenerative cells disclosed herein (e.g., adipose-derived cells such as regenerative cells, and/or microparticles) and/or adipose-derived microparticles can be used to increase the prevalence of M2 macrophages in the CNS in a subject in need thereof, e.g., in a subject that has been diagnosed with or who has been identified as being at risk of traumatic brain injury (including mild, moderate or severe trauma, repeated trauma, and chronic traumatic encephalopathy), PTSD, multiple sclerosis, Alzheimer's disease, ALS, fibromyalgia, Parkinson's disease, Huntington's disease, epilepsy, spinal cord injury, cerebral palsy or the like. Accordingly, some embodiments provide a method of increasing the presence or loco-regional concentration of M2 macrophages in the CNS in a subject that includes the steps of identifying a subject in need of an increase in the presence or amount of M2 macrophages (e.g., by more than 5%, 10%, 15%, 20%, 25, 30%, 50%, or more, compared to untreated subjects) in the CNS, and administering a composition comprising regenerative cells, e.g., adipose-derived cells and/or adipose-derived microparticles as disclosed herein to the subject. In some embodiments, the method further includes the steps of measuring the prevalence of M2 macrophages in the CNS of the subject.

The regenerative cells, e.g., adipose-derived cells (e.g., adipose-derived regenerative cells, such as concentrated populations of adipose-derived cells comprising stem cells) and/or adipose-derived microparticles disclosed herein also manage oxidative stress. Accordingly, the regenerative cells, e.g., adipose-derived cells are useful in treating, inhibiting or ameliorating PTSD and/or the CNS disorders discussed herein associated with oxidative damage to tissue, including, but not limited to traumatic brain injury (including mild, moderate or severe trauma, repeated trauma, and chronic traumatic encephalopathy), PTSD, multiple sclerosis, Alzheimer's disease, ALS, fibromyalgia, Parkinson's disease, Huntington's disease, epilepsy, spinal cord injury, cerebral palsy and the like. For example, the regenerative cells, e.g., adipose-derived cells (e.g., adipose-derived regenerative cells, such as concentrated populations of adipose-derived cells comprising stem cells) can function to scavenge reactive oxygen species and reactive nitrogen species (“RNS”). As used herein, the term “ROS” and “RNS” refer to compounds such as hydrogen peroxide, peroxynitrate, hydroxyl radical (.OH), nitrogen dioxide radical (.NO₂), and carbonate radical (.CO₃). In accordance with some of the embodiments herein, the regenerative cells, e.g., adipose-derived cells (e.g., concentrated populations of adipose-derived cells comprising stem cells), and/or adipose-derived microparticles are used to reduce and/or limit oxidative damage in the CNS. For example, in some embodiments, the regenerative cells, e.g., adipose-derived cells (e.g., concentrated populations of adipose-derived cells comprising stem cells) can be used to reduce oxidative damage in, and/or reduce the concentration of ROS or RNS in the CNS, in a subject in need thereof, e.g., in a subject that has been diagnosed with or who has been identified as being at risk of PTSD, traumatic brain injury (including mild, moderate, or severe trauma, repeated trauma, and chronic traumatic encephalopathy), multiple sclerosis, Alzheimer's disease, ALS, fibromyalgia, Parkinson's disease, Huntington's disease, epilepsy, spinal cord injury, cerebral palsy, or the like. ROS and RNS can be measured using any art-accepted methods now known or discovered in the future, including, for example, the methods described in Halliwell et al. (2004) Br. J Pharmacol. 142:231-255; Tarpee, et al. (2004) Am. J. Physiol. Regul. Integr. Comp. Physiol. 286(3):R431-444. Accordingly, some embodiments provide a method of reducing ROS and/or RNA in the CNS in a subject that includes the steps of identifying a subject in need of reduced ROS and/or RNS levels in the CNS, and administering a composition comprising regenerative cells, e.g., adipose-derived cells (e.g., concentrated populations of adipose-derived cells comprising stem cells) and/or adipose-derived microparticles as disclosed herein to the subject. In some embodiments, the method further includes the steps of measuring the levels of ROS and/or RNS in the CNS of the subject.

Excitotoxicity and oxidative stress cause dendritic and synaptic loss. See, e.g., Hasbani, et al. (2001) J. Neuroci. 2197):2393-24241; Waataja, et al. (2007) J. Neurochem. 104(2): 364-375. Proinflammatory cytokines such as IFN-γ and the like can also synaptic loss, e.g., by altering synaptic proteins (see, e.g., Rao, et al. (2012) Neurochem. Res. 37(5): 903-910) and by causing dendritic retraction (see, e.g., Mizuno, et al. (2008) FASEB J. 22:1797-1806). Matrix metalloproteins (“MMPs”) also play a role in synaptic loss. See, e.g., \check{S}i\check{s}ková, et al. (2013) Neural Plasticity 2013 Article ID 425825. In some embodiments, the regenerative cells, e.g., adipose-derived cells (e.g., concentrated populations of adipose-derived cells comprising stem cells) and/or adipose-derived microparticles are used to reduce or prevent dendritic or synaptic loss (e.g., by virtue of their ability to dampen or reduce excitotoxicity, reduce oxidative stress, and reduce and/or inhibit IFNγ, destructive MMPs, and the like), in a subject in need thereof, e.g., in a subject that has been diagnosed with or who has been identified as being at risk of traumatic brain injury (e.g., mild, moderate, or severe, and/or repeated, including chronic traumatic encephalopathy), PTSD, Multiple sclerosis, Alzheimer's disease, Amyotrophic lateral sclerosis (ALS), Fibromyalgia, Parkinson's disease, Huntington's disease, epilepsy, spinal cord injury, cerebral palsy or the like. Synaptic loss can be measured using any art-accepted method, or any method discovered in the future, including for example, the methods described in Brickman, et al. (2009) Behav. Neurol. 21(1): 93-100; Soricelli et al. (1996) Eur. J. NucL Med. 23(10):1323-1328, and the like. Accordingly, some embodiments provide a method of preventing or inhibiting dendritic and/or synaptic loss in a subject that includes the steps of identifying a subject in need of prevention or inhibition of dendritic and/or synaptic loss, and administering a composition comprising regenerative cells, e.g., adipose-derived cells (e.g., concentrated populations of adipose-derived cells comprising stem cells) and/or adipose-derived microparticles as disclosed herein to the subject. In some embodiments, the method further includes the steps of measuring the levels of dendritic and/or synaptic loss in the subject.

In some embodiments, the regenerative cells, e.g., adipose-derived cells (e.g., adipose-derived regenerative cells, such as concentrated populations of adipose-derived cells comprising stem cells) and/or adipose-derived microparticles disclosed herein are used to reduce lipid peroxidation (e.g., by virtue of their ability to minimize oxidative damage and/or modulate the inflammatory status), in a subject in need thereof, e.g., in a subject that has been diagnosed with or who has been identified as being at risk of traumatic brain injury (including mild, moderate or severe trauma, repeated trauma, and chronic traumatic encephalopathy), PTSD, multiple sclerosis, Alzheimer's disease, Amyotrophic lateral sclerosis (ALS), fibromyalgia, Parkinson's disease, Huntington's disease, epilepsy, spinal cord injury, cerebral palsy or the like. Oxidative damage and lipid peroxidation can be measured using art-recognized methods or methods discovered in the future. By way of example, the methods described in Bosken, et al., “Assessments of Oxidative Damage and Lipid Peroxidation After Traumatic Brain Injury and Spinal Cord Injury” in Animal Models of Acute Neurological Injuries II, Chen, et al. Ed., (c) 2012, Humana Press, New York, N.Y., pp. 347-375; Pratico, et al. (2002) J. Neuro. 80(5): 894-898 can be used to measure lipid peroxidation. As used herein, the term “lipid peroxidation,” or “lipid peroxidation products” or “LPPs” can include, but are not limited to malondialdehyde (MDA) and 4-hydroxynonenal (HNE), acrolein, and the like. Accordingly, some embodiments provide a method of reducing lipid peroxidation in the CNS in a subject in need thereof, that includes the steps of identifying a subject in need of reduced lipid peroxidation in the CNS, and administering a composition comprising regenerative cells, e.g., adipose-derived cells (e.g., adipose-derived regenerative cells, such as concentrated populations of adipose-derived cells comprising stem cells) and/or adipose-derived microparticles as disclosed herein to the subject. In some embodiments, the method further includes the steps of measuring the levels of lipid peroxidation in the CNS of the subject.

As discussed above, neuroexcitotoxcitcity causes a cascade of events leading to apoptosis of neuronal cells. Regenerative cells, e.g., adipose-derived cells (e.g., concentrated populations of adipose-derived cells comprising stem cells) and/or adipose-derived microparticles as disclosed herein secrete and/or increase the concentration of HGF, SCF, FLT-3, SDF-1 (thrombopoietin and IL-3), and other anti-apoptotic factors. Furthermore, increasing the expression of IL-10 can activate CREB and NF-kB, which in turn, can increase expression of various neurotrophins e.g., BDNF, NT3, and NPY. In some embodiments, the regenerative cells, e.g., adipose-derived cells (e.g., adipose-derived regenerative cells, such as concentrated populations of adipose-derived cells comprising stem cells) and/or adipose-derived microparticles are used to prevent, inhibit, or reduce neuronal apoptosis (e.g., via increasing the concentration of, or secreting HGF, SCF, FLT-3, SDF-1, thrombopoietin and IL-3, or the like) in a subject in the CNS of a subject in need thereof, e.g., in a subject that has been diagnosed with or who has been identified as being at risk of traumatic brain injury (mild, severe, and/or repeated, including chronic traumatic encephalopathy), PTSD, Multiple sclerosis, Alzheimer's disease, Amyotrophic lateral sclerosis (ALS), Fibromyalgia, Parkinson's disease, Huntington's disease, epilepsy, spinal cord injury, cerebral palsy, or the like. Neuronal apoptosis can be measured using any art-accepted technique, or any technique discovered in the future. By way of example, non-limiting methods of assessing or measuring neuronal apoptosis useful in the embodiments disclosed herein can are described in Liu, et al. (1997) J. Neurosci. 17(14):5395-5406, Schutte, et al. (1998) J. Neurosci. Methods 86:63-69, and the like. Accordingly, some embodiments provide a method of preventing or inhibiting neuronal apoptosis in a subject that includes the steps of identifying a subject in need of prevention or inhibition of neuronal apoptosis, and administering a composition comprising regenerative cells, e.g., adipose-derived cells (e.g., concentrated populations of adipose-derived cells comprising stem cells) and/or adipose-derived microparticles as disclosed herein to the subject. In some embodiments, the method further includes the steps of measuring the levels of neuronal apoptosis in the subject.

CNS trauma, CNS hypoxia, and neurodegenerative disorders are all characterized by a switch from calcium impermeable to calcium permeable AMPA receptors, worsening the neuroexcitotoxic cascade. Kwak, et al. (2006) Current Opinion in Neurobiology 16(3):281-287. IFN-γ has been demonstrated to effectuate cellular calcium influx, e.g., into neuronal cells, by virtue of the association of AMPA Glu1R receptor subunits and the IFNGR receptor. See, e.g., Mizuno, et al. (2008) FASEB J. 22:1797-1806. Accordingly, in some embodiments, the regenerative cells, e.g., adipose-derived cells (e.g., concentrated populations of adipose-derived cells comprising stem cells), and/or adipose-derived microparticles, can be used to decrease the amount of calcium-permeable AMPA receptors on the post-synaptic cells of the CNS in a subject in need thereof, (e.g., by decreasing and/or dampening IFN-γ or reducing IFN-γ concentration) and/or modulate Ca⁺⁺ influx into post-synaptic cells, e.g., in a subject that has been diagnosed with or who has been identified as being at risk of traumatic brain injury (including mild, moderate or severe trauma, repeated trauma, and chronic traumatic encephalopathy), multiple sclerosis, Alzheimer's disease, ALS, fibromyalgia, Parkinson's disease, and Huntington's disease, epilepsy, spinal cord injury, cerebral palsy or the like. Accordingly, some embodiments provide a method of reducing the number of calcium permeable AMPA receptors in the CNS or modulating Ca⁺⁺ in post-synaptic cells in a subject that includes the steps of identifying a subject in need a reduction in the number of calcium-permeable AMPA receptors, or modulation of Ca⁺⁺ in post-synaptic cells, i.e., a subject having or at risk of developing traumatic brain injury (including mild, moderate or severe trauma, repeated trauma, and chronic traumatic encephalopathy), PTSD, Multiple sclerosis, Alzheimer's disease, Amyotrophic lateral sclerosis (ALS), Fibromyalgia, Parkinson's disease, and Huntington's disease, epilepsy, spinal cord injury, cerebral palsy, or the like, and administering a composition comprising regenerative cells, e.g., adipose-derived cells (e.g., adipose-derived regenerative cells, such as concentrated populations of adipose-derived cells comprising stem cells) and/or adipose-derived microparticles as disclosed herein to the subject. In some embodiments, the method further includes the steps of measuring the levels of calcium permeable AMPA receptors in the subject (e.g., in a sample obtained from the subject).

### Blood Brain Barrier Disruption

The Blood-Brain Barrier (“BBB”) is highly specialized structure of the neurovascular system that separates components of the circulating blood (including plasma components, red blood cells and leukocytes) from neurons and maintains the chemical composition of the neuronal “milieu” which is required for proper functioning of neuronal circuits, synaptic transmission, synaptic remodeling, angiogenesis and neurogenesis in the adult brain. Breakdown and/or disruption of the BBB is associated with CNS diseases and disorders including traumatic brain injury (see, e.g., Barzo, et al. (1996), J. Neurosurg. 85(6):1113-1121) and Marchi, et al. (2013) PLoS ONE 8(3): e56805); Multiple Sclerosis (see, e.g., Bennett et al. (2010) J. Neuroimmunol. 229(1-2):180-91; Alzheimer's Disease and ALS (see, e.g., Sengillo, et al. (2013) Brain Pathol. 23(3):303-310), see also, Zlokovik, et al. (2008) Neuron 57:178-201); Parkinson's Disease (see, e.g., Zlokovik, et al. supra); Huntington's Disease (see, e.g., Browne, et al. (2006), Antioxidants Redox Signal., 8(11-12): 2061-2073); and epilepsy (see, e.g., Oby, et al. (2006) Epilepsia 47(11):1761-1774). Breakdown and/or disruption of the BBB is also associated with PTSD.

The BBB consists of specialized endothelial cells (“cerebral endothelial cells,” or “CEC”), pericytes, and their basal lamina. These cells are surrounded and supported by astrocytes and perivascular macrophages. In contrast to endothelial cells elsewhere in the body, CECs lack fenestration (pores that allow rapid exchange of molecules between vessels and tissue), have few pinocytic vesicles to minimize uptake of extracellular substances, and have extensive tight junctions that severely restrict cell permeability. Limited permeability restricts movement of substances from the systemic circulation to the brain, functioning to buffer the brain from rapid changes in ionic or metabolic conditions. Limited BBB permeability also protects the brain from exposure to molecules that are harmless to peripheral organs but toxic to neurons in the brain. BBB permeability is influenced by neurons, the extracellular matrix, and non-neuronal cells including astrocytes, pericytes, and vascular endothelial cells, e.g., by virtue of cell-cell signaling, signaling molecules, and the like.

Pericytes regulate functional aspects of the BBB, including the formation of tight junctions and vesicle trafficking in CNS endothelial cells. In the BBB, pericytes use finger like projections to wrap around the endothelial cells that line the outside of the capillaries. This allows them to regulate capillary blood flow. Pericytes regulate can cerebral blood flow by inhibiting the expression of molecules that increase vascular permeability and CNS immune cell infiltration. Pericytes also play an active role in BBB functionality by controlling the flow within blood vessels and between blood vessels and the brain. As contractile cells, they can open or close a given amount to allow (or disallow) certain sized particles to flow through the vessel. Such regulation of blood flow is beneficial to neuronal function because it prevents certain particles in the blood from entering the brain. The elasticity of pericytes is beneficial as they are capable of expanding to reduce inflammation and to allow harmful substances to diffuse out of the brain. Pericyte functionality (or dysfunctionality) is implicated in many neurodegenerative diseases such as Alzheimer's (see, e.g., Sengillo, et al. (2013) Brain Pathol. 23:303-310), Parkinson's and ALS (Lou Gehrig's Disease) (see, e.g., Winkler, et al. (2012) J. Cereb. Blood Flow Metab. 32:1841-1852). Specifically, BBB breakdown and toxic extravasation of plasma proteins (e.g. albumin and the like), as well as brain hypoxia, synergistically interact at the neuronal interface and inflict neuronal degenerative changes. See, e.g., Quaegebeur, et al. (2013) Neuron 68: 321-323. BBB integrity can be compromised by loss or dysfunction of pericytes, and degradation and/or loss of the basal lamina and tight junctions.

Under conditions of stress or injury in the CNS, pericytes undergo phenotypic and functional changes such as migration, proliferation and differentiation. Pericyte dysfunction and/or the loss of pericytes in the BBB is likely to play an important role in the pathogenesis of disease. Pericyte loss or a reduced pericyte-to-endothelial cell (EC) ratio in the CNS/BBB may be achieved through: 1) migration of pericytes from their microvascular location; 2) pericyte death; 3) reduced pericyte turnover or maintenance: and 4) selective alteration of pericyte recruitment to EC that may be associated with dysregulation of angiogenesis and abnormal PDGFβ signalling. Pericyte loss leads to brain vascular damage by two parallel pathways: (1) reduction in brain microcirculation causing diminished brain capillary perfusion, cerebral blood flow and cerebral blood flow responses to brain activation leading to chronic perfusion stress and hypoxia, and (2) blood-brain barrier breakdown associated with brain accumulation of serum proteins and several vasculotoxic and/or neurotoxic macromolecules ultimately leading to secondary neuronal degenerative changes. Bell, et al. (2010) Neuron 68(3): 410-427. Platelet-derived growth factor (“PDGF”) functions to recruit pericytes. See, Bonknowski, et al. (2011) Fluids Barriers CNS 8:8. Perictye loss and focal increase in BBB permeability is implicated in many CNS disorders, including TBI (see, e.g., Dore-Duffy (2008) J. Cereb. Blood Flow Metab 26:613-624; (see, e.g., Duz, et al. (2007) Cryobiol. 55:279-284); multiple sclerosis (see, e.g., Bolton, et al. (1997) Mediators Inflamm. 6:295-302), and in Alzheimer's disease (see, e.g., Sagare, et al. (2013) Nat. Comm. 4:2932). Platelet derived growth factor (PDGF) can function to recruit pericytes in the CNS.

Central to BBB integrity is the integrity of the basal lamina and tight junctions. Many of the molecular constituents comprising the cerebrovascular basal lamina and its tight junctions are substrates for matrix metalloproteinase 9 (“MMP-9”). Matrix metalloproteinases are zinc-dependent endopeptidases. MMPs, e.g., MMP-9 are expressed in astrocytes, neurons, activated microglia, endothelial cells and pericytes. As elements of the BBB are substrates for MMPs, MMPs can, and are known to, disrupt BBB integrity. See, Shigemori, et al., (2006) Acta Neurochir Suppl. 96:130-3. While pericytes degrade the components of the extracellular matrix under normal, physiological conditions, increased levels of MMP-9 in the brain are associated with CNS disorders such as Alzheimer's disease (see, e.g., Lorenzl (2003) Neurochem. Int 43(3): 191-196); Parkinson's disease and ALS (see, e.g., He, et al. (2010) PLoSOne 8(9):e73777). In cerebral endothelial cells, astrocytes, microglia and neurons, MMP-9 production is stimulated by pro-inflammatory cytokines including TNF-α.

Cytokines are also known to have a central role in BBB integrity. For example, VEGF is known to enhance post-ischemic BBB integrity. See, e.g., Zecharia et al. (2013) 44(6):1690-1697. By contrast, exposure to pro-inflammatory cytokines, e.g., TNF-α and IFN-γ, have been shown to disrupt the BBB endothelium. See, e.g., Christante, et al. (2012) Proc. Nat. Acad. Sci. USA 110(3): 832-841.

Accordingly, in some embodiments, the regenerative cells, e.g., adipose-derived cells (e.g., concentrated populations of adipose-derived cells comprising stem cells) and/or adipose-derived microparticles can be used to enhance or improve BBB integrity (e.g., by modulating BBB permeability, reducing BBB leakage, reducing improper infiltration of cells and/or other elements into the BBB) in a subject in need thereof, (e.g., by secreting or increasing the concentration of VEGF, PDGF, and/or dampening TNF-α and/or IFN-γ, and/or inhibiting MMPs, and/or providing pericytes, endothelial precursor cells and the like) in a subject that has been diagnosed with or who has been identified as being at risk of traumatic brain injury (including mild, moderate, or severe trauma, repeated trauma, and chronic traumatic encephalopathy), PTSD, multiple sclerosis, Alzheimer's disease, ALS, fibromyalgia, Parkinson's disease, Huntington's disease, epilepsy, spinal cord injury, cerebral palsy or the like. BBB integrity can be measured using any art-accepted technique, including but not limited to the methods described in Kassner, et al. “Measuring the Integrity of the Human Blood-Brain Barrier Using Magnetic Resonance Imaging,” in The Blood-Brain and Other Neural Barriers: Reviews and Protocols, Nag, ed. © 2011, Springer Science & Business Media, LLC, Philadelphia, Pa., pp. 229-245. Accordingly, some embodiments provide a method of enhancing BBB integrity in a subject that includes the steps of identifying a subject in need of enhancement of BBB integrity, and administering a composition comprising regenerative cells, e.g., adipose adipose-derived cells (e.g., adipose-derived regenerative cells, such as concentrated populations of adipose-derived cells comprising stem cells) and/or adipose-derived microparticles as disclosed herein to the subject. In some embodiments, the method further includes the steps of measuring the BBB integrity in the subject.

In some embodiments, the regenerative cells, e.g., adipose-derived cells (e.g., adipose-derived regenerative cells, such as concentrated populations of adipose-derived cells comprising stem cells) are used to increase the number of pericytes and/or pericyte coverage and or pericyte:endothelial cell ratio in the BBB in a subject in need thereof, e.g., in a subject that has been diagnosed with or who has been identified as being at risk of traumatic brain injury (including mild, moderate, or, severe trauma, repeated trauma, and chronic traumatic encephalopathy), PTSD, multiple sclerosis, Alzheimer's disease, ALS, fibromyalgia, Parkinson's disease, Huntington's disease, epilepsy, cerebral palsy, or the like. Extravascular levels of IgG and fibrin (two plasma-derived proteins) in the brain are correlated with a deficiency in pericyte coverage of brain capillaries. Accordingly, pericyte coverage and number in the brain, and the extent of capillary leakage or BBB breakdown, can be assessed using any art-accepted means, including but not limited to measuring extravascular IgG levels and/or fibrin levels, e.g., as described in Sengillo, et al. (2012) Brain Pathol. 23: 303-310. According to some of the embodiments disclosed herein, provided is a method of increasing the number of perictyes or pericyte coverage in the BBB in a subject in need thereof, that includes the steps of identifying a subject in need of increased pericyte number or coverage in the BBB, and/or enhanced BBB integrity, and administering a composition comprising regenerative cells, e.g., adipose-derived cells (e.g., adipose-derived regenerative cells, such as concentrated populations of adipose-derived cells comprising stem cells) and/or adipose-derived microparticles disclosed herein to the subject. In some embodiments, the methods further include a step of measuring pericyte coverage and/or pericyte number in the subject.

In some embodiments, the regenerative cells, e.g., adipose-derived cells (e.g., adipose-derived regenerative cells, such as concentrated populations of adipose-derived cells comprising stem cells) and/or adipose-derived microparticles are used to improve or enhance cerebral blood flow (“CBF”) in a subject in need thereof. Defects in cerebral blood flow are associated with TBI (Kelly, et al. (1997) J. Nuero. 86(4): 633-641); PTSD and trauma to the brain, multiple sclerosis (Ota, et al. (2013) Magn. Res. Imaging 31(6): 990-995); Alzheimer's Disease (see, e.g., Firbank, et al. (2003) Neuroimage 20(2): 1309-1319); Parkinson's Disease (see, e.g., Firbank, supra), ALS (see, e.g., Tanaka et al. (2003) Neurol. Res. 25(4): 351-356); Huntington's disease (see, e.g., Hasselbach, et al. (1993) Neurol Neurosurg Psychiatry 55:1018-1023), and the like. For example, in some embodiments, regenerative cells, e.g., adipose-derived regenerative cells (e.g., adipose-derived stem cells, endothelial precursor cells, endothelial progenitor cells, endothelial cells, pericytes, and the like) and/or adipose-derived microparticles are used to restore of CBF in subjects in need thereof e.g., in a subject that has been diagnosed with or who has been identified as being at risk of traumatic brain injury (including mild, moderate, or, severe trauma, repeated trauma, and chronic traumatic encephalopathy), multiple sclerosis, Alzheimer's disease, ALS, Parkinson's disease, and Huntington's disease, epilepsy, cerebral palsy or the like. Cerebral blood flow can be measured using any art-accepted technique, including but not limited to, e.g., single-photon emission computed tomography (“SPECT”), or the like. Accordingly, some embodiments provide a method for improving or enhancing cerebral blood flow in a subject in need thereof, e.g., in a subject that has been diagnosed with or who has been identified as being at risk of traumatic brain injury (including mild, moderate, or severe trauma, repeated trauma, and chronic traumatic encephalopathy), PTSD, multiple sclerosis, Alzheimer's disease, ALS, fibromyalgia, Parkinson's disease, and Huntington's disease, epilepsy, cerebral palsy or the like, that includes the steps of identifying a subject in need of enhanced or improved cerebral blood flow, and administering to the subject a composition comprising the regenerative cells, e.g., adipose-derived cells (e.g., adipose-derived regenerative cells, such as concentrated populations of adipose-derived cells comprising stem cells) and/or adipose-derived microparticles disclosed herein. The method can also include the step of measuring the cerebral bloodflow of the subject.

Compromise of the BBB can lead to lymphocytic infiltration into the brain. Lymphocytic infiltration has been implicated in the progression of Parkinson's disease (see, Hirsch et al. (2009) Lancet Neurol. 8:382-397, Dos Santos et. al. (2008) J Neuroinflammation 5:49); multiple sclerosis (see, e.g., Deloire (2004) Mult. Scler. 10(50: 540-548); Alzheimer's disease, and epilepsy (see, e.g. Rossi, et al. (2011) J. Leuk. Biol. 89(4): 539-559). Regenerative cells, e.g., adipose-derived cells (e.g., adipose-derived regenerative cells, such as concentrated populations of adipose-derived cells comprising stem cells) and/or adipose-derived microparticles as described herein can prevent, inhibit or reduce leukocyte infiltration across the blood brain barrier. Accordingly, some embodiments provide a method of inhibiting lymphocytic infiltration across the blood brain barrier in a subject in need thereof, e.g., a subject that has been identified as having or who has been identified as being at risk of traumatic brain injury (including mild, moderate, or severe trauma, repeated trauma, and chronic traumatic encephalopathy), PTSD, multiple sclerosis, Alzheimer's disease, ALS, fibromyalgia, Parkinson's disease, and Huntington's disease, epilepsy, cerebral palsy or the like, that includes the steps of identifying a subject in need of prevention or inhibition of lymphocytic infiltration across the BBB, and administering to the subject a composition comprising the regenerative cells, e.g., adipose-derived cells (e.g., adipose-derived regenerative cells, such as concentrated populations of adipose-derived cells comprising stem cells) and/or adipose-derived microparticles disclosed herein. In some embodiments, the methods can further include the step of measuring or determining lymphocytic infiltration into the brain by any art-accepted technique.

### Cerebral Metabolism

The brain is metabolically one of the most active of all organs in the body. Not only does the brain utilize O₂ at a very rapid rate, but it is absolutely dependent on uninterrupted oxidative metabolism for maintenance of its functional and structural integrity. Regulation of cerebral blood flow is achieved mainly by control of the tone or the degree of constriction, or dilation, of the cerebral vessels. See, Clarke, et al. “Regulation of Cerebral Metabolism,” in Basic Neurochemistry Molecular, Cellular and Medical Aspects, 6^{th} Ed., Siegel G J, Ed. (c) 1999, Lippincott Raven, Philadelphia, Pa., Ch. 31. Reduced cerebral metabolism, i.e., a depressed level of cerebral glucose uptake in the brain, is associated with several CNS disorders and conditions. For example, there is a transient increase in cerebral glucose intake in the brain immediately after traumatic brain injury, which is followed by a prolonged period of depression of the glucose metabolism. See, e.g., Prins et al. (2013) J. Neurotrauma 30:30-38. Reduced cerebral metabolism, i.e., a depressed level of cerebral glucose uptake in the brain, is associated with several CNS disorders and conditions, including PTSD and trauma. See, e.g., Kim, et al. (2012) Neuroimaging 301(3):214-217; see also, Prins et al. (2013) J. Neurotrauma 30:30-38. Similarly, decreased cerebral metabolism is observed in subjects with amyotrophic lateral sclerosis (see, Marinos, et al. (2004) Ann. Neurol. 22(5):580-586; Parkinson's disease (see, e.g., Bohnen, et al. (2011) J. Nucl. Med. 52: 848-855; Alzheimer's disease (see, e.g., McGreer, et al. (1986) Can. Med. Assn. J. 134: 597-607); (see, e.g., Pusinelli, et al. (1982) Ann. Neurobiol. 11(5): 499-509); multiple sclerosis (see, e.g., Roelcke, et al. (1997) Neurol. 48(6): 1566-1571 epilepsy (see, e.g., Engel, et al. (1982) Science 218(4567): 64-66), and dementia (see, e.g., Meguro, et al. (1991) Neuroradiol. 33:305-306, Ishii, et al. (2014) Neuroradiol. 10.3174.ajnr.A3695).

In some embodiments, the adipose-derived cells (e.g., adipose-derived regenerative cells, such as concentrated populations of adipose-derived cells comprising stem cells) and/or adipose-derived microparticles are used to increase cerebral glucose metabolism, e.g., in a subject in need thereof, e.g., a subject that has suffered traumatic brain injury, a subject that has been diagnosed with or who has been identified as being at risk of traumatic brain injury (including mild, moderate or severe trauma, repeated trauma, and chronic traumatic encephalopathy), PTSD, Multiple sclerosis, Alzheimer's disease, Amyotrophic lateral sclerosis (ALS), Fibromyalgia, Parkinson's disease, and Huntington's disease, epilepsy, or the like. For example, in some embodiments, an improvement in cerebral metabolism of greater than 10%, 15% 20%, 25%, 30%, or more, in subjects in need thereof, is observed following administration of the regenerative cells and/or adipose-derived microparticles disclosed herein. Cerebral glucose metabolism can be readily measured using art-accepted techniques, such as positron emission tomography using [¹⁸F]-2-fluoro-2-doexyglucose, as described in Ishii, et al. supra, and references cited therein. Accordingly, in some embodiments, a method is provided wherein a subject in need of increased cerebral glucose metabolism is identified, and administered a composition comprising regenerative cells e.g., adipose-derived regenerative cells, such as concentrated populations of adipose-derived cells comprising stem cells) and/or adipose-derived microparticles, as disclosed herein. In some embodiments, the method can further include the step of measuring cerebral glucose metabolism.

### Diseases and Disorders of the CNS

### Traumatic Brain Injury (“TBI”) and Chronic Traumatic Encephalopathy (“CTE”)

Embodiments disclosed herein relate to the treatment, prevention, and amelioration of traumatic brain injury (“TBI”). Traumatic brain injury is a form of acquired brain injury that occurs when a sudden trauma causes damage to the brain. In the United States alone, approximately 1.5 million individuals experience TBI, although the number of unreported injuries is much higher. TBI can be mild, moderate or severe. While some symptoms appear immediately, others do not appear until days, weeks, months or even years after the TBI event(s). Symptoms of mild TBI include headache, confusion, dizziness, blurred vision, changes in mood, and impairment in cognitive function, such as memory, learning, and attention. Symptoms of moderate to severe TBI include, in addition to those observed for mild TBI, nausea, convulsions or seizures, slurring of speech, numbness of extremities, and loss of coordination.

TBI is a complex pathological process that involves three overlapping phases: primary injury to brain tissue and cerebral vasculature caused by the initial impact to the head, secondary injury including neuroinflammatory processes triggered by the primary insult. The tertiary phase of TBI includes ongoing abnormalities in glucose utilization, cellular metabolism, as well as membrane fluidity, synaptic function, and structural integrity See, e.g., Hovda, et al. (2007) Crit Care Med. 35:663-664; Aoyama et al, (2008) Brain Res. 1230:310-319.

In certain methods disclosed herein, a subject having TBI is identified. TBI can be diagnosed using any art-accepted method including, but not limited to asses a subject's physical injuries, brain and nerve functioning, and level of consciousness. Tests useful in identifying individuals that have suffered traumatic brain injury include the Glascow Coma Scale (“GCS”). The GCS measures a person's functioning in three areas: (1) speech, e.g., whether the person speaks normally, speaks in a way that doesn't make sense, or doesn't speak at all; (2) ability to open eyes, e.g., whether the person opens his or her eyes only when asked; (3) ability to move, ranging from moving one's arms easily to not moving even in response to painful stimulation. A health care provider rates a person's responses in these categories and calculates a total score. A score of 13 and higher indicates a mild TBI, 9 through 12 indicates a moderate TBI, and 8 or below indicates severe TBI. Subjects having TBI can also be identified by measuring intracranial pressure (“ICP”) using art accepted techniques. Cognition and neuropsychological assessments, including but not limited to Frontal Assessment Battery (see, e.g., Dubois et al. (2000) Neurology 55:1621-6) and the Behavioral Dyscontrol Scale (Kaye, et al. (1990) J Am Geriatr. Soc. 38:1304-1310), and the like, are useful in detection TBI, including mild, moderate and severe TBI in subjects.

The American Congress of Rehabilitation Medicine (“ACRM”) has defined mild TBI as the occurrence of any one of the following symptoms following external application of force to the brain: any period of loss of consciousness, any loss of memory for events immediately before (retrograde amnesia) or after (anterograde amnesia) the accident (collectively referred to as the period of post-traumatic amnesia, or PTA), any alteration in mental state at the time of the accident (e.g., feeling dazed, disoriented, or confused), or focal neurologic deficit(s) that may or may not be transient. The ACRM definition of mild TBI includes only those injuries in which loss of consciousness is 30 minutes or less, the GCS score at 30 minutes after injury is 13-15, and the duration of PTA is no longer than 24 hours. Injuries exceeding these criteria are considered to be of more than mild severity.

Imaging techniques such as computerized tomography (“CT”) scan, magnetic resonance imaging (“MRI”), including functional MRI (“fMRI”), proton or phosphorous magnetic resonance spectroscopy (MRS), Single photon computed tomography (SPECT), positron emission tomography (PET), are also useful in identifying subjects with TBI in the embodiments disclosed herein. Measurement of cerebral glucose metabolism, e.g., as used in the diagnosis of or identification of subjects with TBI in the embodiments disclosed herein.

Some embodiments include the identification of subjects that are “at risk of TBI” include, but are not limited to, a subject participating in a sport with occurrence of concussions. Exemplary subjects in this category include, among others, football players, boxers, soccer players, and hockey players, a combatant in an armed conflict, for example, a soldier, or a subject undergoing brain surgery.

The regenerative cells (e.g., adipose-derived regenerative cells, such as concentrated populations of adipose-derived cells comprising stem cells) and/or adipose-derived microparticles can be administered according to any of the methods disclosed herein. By way of example, according to certain embodiments, the compositions comprising regenerative cells disclosed herein are administered to a subject suffering a TBI or at risk of TBI within 30 days, within 28 days, within 14 days, within 7 days, within 6 days, within 5 days, within 4 days, within 3 days, within 2 days, within 24 hours, within 12 hours, within 11 hours, within 9 hours, within 8 hours, within 7 hours, within 6 hours, within 5 hours, within 4 hours, within 3 hours, within 2 hours, or within one hour, of a traumatic brain injury, according to any one or more of the methods of administration discussed below.

In some embodiments, the subject is administered more than one dose of regenerative cells (e.g., adipose-derived regenerative cells, such as concentrated populations of adipose-derived cells comprising stem cells) and/or adipose-derived microparticles as disclosed herein. By way of example only, in some embodiments, a subject is administered daily doses of the compositions disclosed herein over a period of time, e.g., at regular or irregular intervals. For example, in some embodiments, the subject is administered a composition as disclosed herein daily over a period of 2, 3, 4, 5, 6, 7, 8, 9, 10, 11, 12, 13, 14, or more days. In some embodiments, the subject is administered sequential doses of the compositions disclosed herein, e.g., at regular or irregular intervals until the subject exhibits normal cerebral glucose metabolism.

In some embodiments, the regenerative cells (e.g., adipose-derived regenerative cells, such as concentrated populations of adipose-derived cells comprising stem cells) e.g., adipose-derived regenerative cells, such as concentrated populations of adipose-derived cells comprising stem cells) and/or adipose-derived microparticles disclosed herein reduce or ameliorate the physiological effects of, or shorten the time period of, the secondary injury phase of TBI (e.g., reduce or ameliorate one more aspects of neuroexcitotoxcity and neuroinflammation, BBB alterations, cerebral metabolism and cerebral blood flow, and the like, discussed elsewhere herein). The compositions and methods disclosed herein are useful in reducing or modulating microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improving cerebral metabolism in subjects identified as having or at risk of having one or more TBI events.

In some embodiments, following administration of the regenerative cells (e.g., adipose-derived regenerative cells, such as concentrated populations of adipose-derived cells comprising stem cells) and/or adipose-derived microparticles disclosed herein, a subject's functional outcome is improved, the subject's probability of survival is increased, the oxygen tension in a neuronal tissue of the central nervous system of the subject is increased, the progression of damage to, or ischemic damage to, secondary ischemic damage to the central nervous system of the subject is reduced, and/or the loss of neuronal tissue in the central nervous system of the subject is reduced. In some embodiments, treatment improvement can be characterized as an increase in either the rate and/or the extent of behavioral and anatomical recovery following TBI.

Also provided are methods and compositions useful for reducing vulnerability to secondary brain insult in a subject in need thereof in a subject in need thereof. Among the 3.5 million annual new head injury cases is a subpopulation of repeated traumatic injury (“rTBI”) cases. Damage from successive TBI events is cumulative. See, e.g., Cantu, et al. (1998) Clinics in Sports Med. 17(1):37-44, 1998. When a second concussion occurs prior to recovery from the first, rapid onset of cerebral edema and death can occur, particularly for athletes prematurely returning to play. Changes in cerebral glucose metabolism (“CMRglc”) occur after TBI, including mild, moderate and severe TBI. Depressed levels of CMRglc are directly correlated with TBI severity and outcome measures, and the presence of CMRglc depression has been shown to reflect an altered, “vulnerable” cerebral state, during which secondary TBI generates a significantly worsened outcome compared to a secondary TBI occurring outside of the window of cerebral vulnerability. Prins, et al. (2013) J. Neurotrauma 30(1):30-38. As used herein, the phrase “reducing vulnerability to secondary brain insult” refers to a reduction in, and/or improvement of, any one or more physiological effects of a brain injury that may exacerbate a subsequent traumatic insult to the brain or CNS, including but not limited to, reduction in excitotoxicity and neuroinflammation as discussed above, restoration of cerebral metabolism and cerebral blood flow, restoration of BBB integrity, and the like, as described elsewhere herein following a TBI. In some embodiments, “reducing vulnerability to secondary brain injury” comprises restoring CMRglc to pre-TBI levels. In some embodiments, “reducing vulnerability to secondary brain injury” refers to decreasing the time period, e.g., by hours or days, that CMRglc is depressed following TBI. CMRglc can be readily analyzed using the methods described in Ishii, et al. supra, Cohen, et al. (2002) Magn. Reson. Med. 48(6): 1063-1067, or other art-accepted means. Accordingly, some embodiments relate to methods of reducing vulnerability to secondary brain insult in a subject in need thereof, that include a step of identifying a subject in need of a reduction to vulnerability to secondary injury in a subject in need thereof, and administering a composition comprising the adipose-derived cells (e.g., adipose-derived regenerative cells, such as concentrated populations of adipose-derived cells comprising stem cells) as disclosed herein. In some embodiments, the methods disclosed herein include the step of determining CMRglc levels.

Some embodiments provide methods and compositions for mitigating the effects of secondary brain insult in a subject in need thereof. For example, as discussed above, individuals suffering multiple traumatic brain injury events have worsened outcomes associated with subsequent injury. In a similar token, individuals that suffer one or more traumatic brain events are more likely to develop other CNS diseases and disorders such as Alzheimer's disease, Parkinson's disease, epilepsy, and the like. Accordingly, some embodiments disclosed herein relate to mitigating the effects of secondary brain insult, e.g., improving outcome associated with a subsequent brain injury, lessening the likelihood of developing one or more of Alzheimer's disease, Parkinson's disease, epilepsy and the like, in subjects that have experienced a traumatic brain insult, by administering to the subject a composition comprising regenerative cells (e.g., adipose-derived regenerative cells, such as concentrated populations of adipose-derived cells comprising stem cells) and/or adipose-derived microparticles.

Provided herein are methods for the treatment or prevention of chronic traumatic encephalopathy (“CTE”) or “dementia pugilistica (“DP”), e.g. prevention of the onset of CTE/DP, reduction of the risk of developing CTE/DP, or the reduction and/or amelioration one or more symptoms associated with CTE/DP. CTE/DP is a neurodegenerative disorder occurring in individuals with a history of multiple concussions and other forms of head injury. CTE/DP is most common in professional athletes participating in football, ice hockey, professional wrestling and other contact sports who have experienced repetitive brain trauma. CTE/DP is also reported in soldiers exposed to a blast or a concussive injury. Physiological signs of CTE/DP include brain tissue degeneration and the accumulation of tau protein, beta amyloid plaques, and reduction in brain weight. Individuals with CTE often show symptoms of dementia, such as memory loss, aggression, confusion and depression, which generally appear years or many decades after the trauma.

Some of the methods disclosed herein include the step of identifying subjects that have or that are at risk of developing CTE/DP. Methods to identify individuals with CTE/DP or who are at risk of developing CT/DP include, but are not limited to, Positron Emission Tomography (PET [¹⁸F]FDDNP. See, e.g., Zhang, et al. (2012) J. Alzheimer's Dis 31 (3): 601-612; Small, et al. (2013) Am. J. Geriatric Psych. 21 (2): 138-144. As CTE/DP is a result of rTBI, in some embodiments, identifying a subject at risk of TBI, and at risk of rTBI as discussed above.

For reasons outlined above, the regenerative cells (e.g., adipose-derived regenerative cells, such as concentrated populations of adipose-derived cells comprising stem cells) and/or adipose-derived microparticles disclosed herein are useful in ameliorating, treating, or preventing CTE/DP. Specifically, the compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibit neuronal apoptosis, prevent or inhibit dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing CTE/DP. Accordingly, some embodiments provide a method of treating or preventing CTE/DP that includes the step of identifying an individual that has or is at risk of developing CTE/DP, and administering a composition comprising regenerative cells (e.g., adipose-derived regenerative cells, such as concentrated populations of adipose-derived cells comprising stem cells) and/or adipose-derived microparticles as disclosed herein to the subject. In some embodiments, the subject will show improvement in cognitive function, psychological well-being (e.g., decreased incidence of depression, aggression, and the like), motor function (e.g., Parkinsonian-like symptoms associated with CTE/DP), and the like.

### Amyotrophic Lateral Sclerosis (“ALS”)

Provided herein are methods and compositions useful for the inhibition of, prevention of, or treatment of amyotrophic lateral sclerosis (“ALS”). ALS is a condition in which cell death of spinal cord motor neurons leads to loss of function of nerves which control muscular contraction, thereby preventing movement of muscles throughout the body (including muscles involved in respiration) and leading to death of the patient within 2-3 years after onset.

Currently physicians have limited choices for treating ALS. At this time, riluzole is the only drug that has been approved by the FDA for treatment of ALS. In clinical trials, riluzole has shown only a slight benefit in modestly increasing survival time. Thus there is an urgent need for effective therapies for ALS.

The onset of ALS is a non symptomatic stage when there is retraction of motor axons from their synapses onto muscles. Glutamate excitotoxicity and neuroinflammation have been identified with onset of ALS. See, e.g., Appel, et al., (2009) Trends Inmmunol. 31(1):7-17. During the symptomatic phase of ALS, unknown mechanisms result in deleterious immune response with subsequent neuroinflammation and neurodegeneration. The symptomatic phase of ALS is characterized by damage to microglia and astrocytes, loss of muscle strength and slurred speech. In the final stages of the disease, subjects exhibit paralysis and muscle atrophy.

In some embodiments disclosed herein, subjects are identified as having or at risk of developing ALS. Any art-recognized method can be used in the embodiments disclosed herein to identify subject that have or that are at risk of developing ALS. For example, standard criteria for diagnosis of ALS have been established by the World Federation of Neurology, and are described in Brooks et al. (2000) Amyotroph. Lateral. Scler. Other Motor Neuron Disord. 1(5):293-299. The “El Escorial” criteria for the diagnosis of ALS require: (1) the presence of (a) evidence of lower motor neuron (LMN) degeneration by clinical, electrophysiological or neuropathologic examination; (b) evidence of upper motor neuron (UMN) degeneration by clinical examination; and (c) a progressive spread of symptoms or signs within a region or to other regions as determined by history or examination; and (2) the absence of (a) electrophysiological or pathological evidence of other disease processes that might explain the signs of LMN or UMN degeneration; and (b) neuroimaging evidence of other disease processes that might explain the observed clinical and electrophysiological signs.

Subjects at risk of developing ALS can be identified using art-recognized methods that assess molecular markers indicative of or associated with ALS. By way of example only, subjects at risk of developing ALS can be identified by determining if the subject tests positive for any one of the known mutations in the SOD1 gene described in, for example, Deng et al. (1993), Science, 261:1047-1051.

One or more of the following standard clinical evaluations can be used to identify ALS symptoms or assess progress/prevention of ALS in a subject:

(1) Quantitative strength and functional markers. The TUFTS Quantitative Neuromuscular Examination (“TQNE”) is a well standardized, reliable, validated test to measure strength and function in ALS. The test involves measurement of maximum voluntary isometric contraction (MVIC) of eight muscle groups in the arms using a strain gauge tensiometer.

(2) Functional measures. The ALS Functional Rating Scale (“ALSFRS”) is an easily administered ordinal rating scale used to determine patient′ assessment of their ability and independence for ten functional activities. Validity has been established by correlating ALSFRS scores with change in strength over time.

(3) Neuropathologic examination of muscle, peripheral nerve and other tissue biopsies. The presence of neuropathologic features such as chronic denervation/reinervation in affected muscle tissue, scattered hypertrophied muscle fibers, necrotic muscle fibers, inflammatory cell infiltration and giant axonal swellings in intramuscular nerves are all indicative of ALS. An overview of neuropathologic findings in patients with ALS is presented in, for example, Hirano, et al. (1996) Neurolog, 47 (Suppl. 2): S63-S66.

In some embodiments, the regenerative cells (e.g., adipose-derived regenerative cells, such as concentrated populations of adipose-derived cells comprising stem cells) and/or adipose-derived microparticles disclosed herein reduce or ameliorate physiological features of ALS (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibit neuronal apoptosis, prevent or inhibit dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing ALS.

In some embodiments, a subject's functional outcome is improved following administration of the regenerative cells (e.g., adipose-derived regenerative cells, such as concentrated populations of adipose-derived cells comprising stem cells) and/or adipose-derived microparticles as disclosed herein. In some embodiments, the subject's probability of survival is increased following administration of the adipose-derived regenerative cells as disclosed herein. In some embodiments, treatment improvement can be characterized as an increase in either the rate and/or the extent of behavioral and anatomical recovery, or the slowing of disease progression. For example, in some embodiments, administration of the regenerative cells as disclosed herein to a subject that has or that is at risk of developing ALS, reduces or inhibits axonal demyelination, reduces the degeneration of motor neurons associated with ALS, treats or reduces paralysis and/or spread of paralysis associated with ALS, alleviates tremor associated with ALS, and the like.

### Parkinson's Disease (“PD”)

Parkinson's disease (PD) is a chronic and progressive degenerative disease of the brain that impairs motor control, speech, and other functions. Physical manifestations of Parkinson's disease include (1) a slowing down of all movements (bradykinesia), quiet and monotonous speech (akinesia or hypokinesia), absence of the physiological associated movements, a stooped posture, a small-step, partially shuffling gait, handwriting which becomes smaller as the writing continues, uncontrollable disturbances in movement, with a tendency to fall forward to the side or backward, (2) rigidity of the musculature (rigor), and (3) coarse resting tremor (trembling). Parkinson's disease is a disease that occurs relatively frequently and develops in approx. 1% of individuals aged over 60, in particular in men.

Parkinson's disease is characterized by neuronal death, including a group of neurons that synthesize the neurotransmitter dopamine (“DA”) located in the substantia nigra of the midbrain. The loss of DA in this area of the brain results in most of the motor symptoms of Parkinson's disease. Non-dopaminergic neurons, including norepinephrinergic neurons, cholinergic, and serotoninergic neurons are also affected in Parkinson's disease. In addition to the neuronal loss, Parkinson's disease is pathologically characterized by the presence of proteinaceous inclusions, such as Lewy bodies or Lewy neurites. Neuroexcitotoxicity and neuroinflammation, as well as disruption in the BBB and cerebral metabolism/cerebral blood flow have been implicated in the pathophysiology of Parkinson's Disease. See, e.g., Zlokovik, et al. supra; Hirsch, et al. (2009) Lancet Neurol. 8:382-397, Dunnett, et al. (1999) Nature 399: A32-A39; Olanow and Tatton (1999) Annu Rev. Neurosci. 22: 123-144.

In some embodiments disclosed herein, subjects are identified as having or at risk of developing Parkinson's disease. Any art-accepted method can be used in the embodiments disclosed herein to identify subjects that have or that are at risk of developing Parkinson's disease. By way of example only, as Parkinson's disease manifests in a pattern of reduced dopaminergic activity in the basal ganglia a subject that has or is at risk of developing Parkinson's disease can be identified by reduced dopaminergic activity in the basal ganglia, e.g., using art-recognized methods such as molecular scanning using DaTscan (SPECT scanning using 123-ioflupane), PET scanning using fluorine-18-dihydroxyphenylalanine, and the like.

Other non-limiting examples of art-accepted methods used in the diagnosis and outcome measures of Parkinson's disease useful in the embodiments include, for example, Physician's assessments of a subject's physical examination scored with the Unified Parkinson's Disease Rating Scale (UPDRS) (see, Fahn, et al. “UPDRS Development Committee. Unified Parkinson's disease rating scale”, in Recent Developments in Parkinson's Disease, Fahn, Ed. New York: Macmillan, 1987, 153-167); and the modified Hoehn and Yahr (H&Y) staging scale (Hoehn, et al. (1967) Neurology 17(5), 427-442). Other exemplary methods to identify a subject that has or is at risk of developing Parkinson's disease useful in the embodiments disclosed herein include, but are not limited to, testing for one or more biomarkers according to methods known to those skilled in the art. See, e.g., Molochnikov, et al., (2012) Mol Neurodegener. 31; 7:26; Scherzer, et al. (2007) Proc Natl Acad Sci USA. 104(3):955-60, European Patent Application Publication No. EP 2633078, and the like.

In some embodiments, the regenerative cells (e.g., adipose-derived regenerative cells, such as concentrated populations of adipose-derived cells comprising stem cells) and/or adipose-derived microparticles disclosed herein, reduce or ameliorate physiological features of Parkinson's (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing Parkinson's.

In some embodiments, the administration of the regenerative cells (e.g., adipose-derived regenerative cells, such as concentrated populations of adipose-derived cells comprising stem cells) and/or adipose-derived microparticles disclosed herein will effectively reduce or ameliorate one or more of the symptoms of Parkinson's, or the progression of the disease will be retarded (e.g., the subject's condition will have stabilized). In some embodiments, following treatment with the adipose-derived regenerative cells, the subject will exhibit sparing of pre- or postsynaptic dopaminergic terminals, e.g., as assessed by MRI, SPECT, or PET as discussed elsewhere herein.

### Alzheimer's Disease (“AD”)

Alzheimer's disease (“AD”) is one of the most common causes of dementia, affecting more than 5 million individuals in the United States alone. Alzheimer's Disease causes the death of several types of neuronal lineage cells within multiple regions of the brain, specifically cholinergic neurons. Hallmarks of Alzheimer's disease include intracellular neurofibrillary tangles and extracellular plaques (“Aβ plaques”) containing deposits of beta-amyloid protein. Aβ plaques are generated by γ- and β-secretases that cleave the amyloid precursor protein (“APP”) at specific amino acids. Neurofibrillary tangles are composed of tau proteins that are hyper-phosphorylated, resulting in neuron impairment. Both of these hallmarks lead to cognitive impairment and loss of memory. Currently, there is no treatment available for AD. Drugs used in the management of AD offer purely symptomatic relief.

Although the etiology of Alzheimer's disease is not completely understood, it is known that toxic buildup of beta-amyloid (Aβ) production and accumulation are associated with microglial activation, that leads to a vicious cycle of inflammation formed between Aβ accumulation, activated microglia, and microglial inflammatory mediators, which themselves further drive and enhance Aβ deposition and neuroinflammation.

According to some embodiments disclosed herein, provided are compositions and methods for the treatment or prevention of AD in a subject in need thereof. Accordingly, in some embodiments, a subject that has or is at risk of developing Alzheimer's disease is identified. In Alzheimer's disease, eight cognitive domains are most commonly impaired, including memory, language, perceptual skills, attention, constructive abilities, orientation, problem solving and functional abilities. Accordingly, the identification of a subject that has or is at risk of developing Alzheimer's disease can include measurement of any of the aforementioned cognitive indices, using any art-accepted tests.

A technique known as PiB PET has been developed for directly and clearly imaging beta-amyloid deposits in vivo using a tracer that binds selectively to the A-beta deposits. The PiB-PET compound uses carbon-11 PET scanning. Recent studies suggest that PiB-PET is 86% accurate in predicting which people with mild cognitive impairment will develop Alzheimer's disease within two years, and 92% accurate in ruling out the likelihood of developing Alzheimer's. A similar PET scanning radiopharmaceutical compound called (E)-4-(2-(6-(2-(2-(2-([¹⁸F])-fluoroethoxy)ethoxy)ethoxy)pyridin-3-yl)vinyl)-N-methyl benzenamine, or ¹⁸F AV-45, or florbetapir-fluorine-18, or simply florbetapir, contains the longer-lasting radionuclide fluorine-18, has recently been created, and tested as a possible diagnostic tool in Alzheimer's patients. Florbetapir, like PiB, binds to beta-amyloid, but due to its use of fluorine-18 has a half-life of 110 minutes, in contrast to PiB's radioactive half life of 20 minutes. Thus, in some embodiments, a subject that has or is at risk of developing Alzheimer's disease is identified as a subject having increased a-beta deposits, using PET scanning with any of the aforementioned compounds.

Volumetric MRI can detect changes in the size of brain regions. Measuring regions that atrophy during the progress of Alzheimer's disease is showing promise as a diagnostic indicator. Thus, other non-useful means of identification of subjects that are at risk of developing Alzheimer's disease is the determination of the presence of atrophic brain region(s) in the subject, e.g., using any art-recognized method.

Another recent objective marker of Alzheimer's disease useful in the identification of subject that have or that are at risk of developing Alzheimer's disease include, but are not limited to the analysis of amyloid beta or tau proteins, both total tau protein and phosphorylated tau_{181P} protein concentrations in the cerebrospinal fluid. Identification of these proteins using a spinal tap can predict the onset of Alzheimer's with a sensitivity of between 94% and 100%. Thus, subjects with elevated levels of tau and/or amyloid beta proteins in their cerebral spinal fluid can be identified as subjects at risk of developing Alzheimer's. When used in conjunction with existing neuroimaging, doctors can identify patients with significant memory loss who are already developing the disease. In some embodiments, a subject that has or is at risk of developing Alzheimer's disease can be identified using art-recognized molecular markers. By way of example only, in some embodiments, identification can be made using the methods described in Carrette, et al. (2003) Proteinomics 3(8):1486-1494.

Various other non-limiting methods for the identification of subjects that have or that are at risk of developing AD useful in the embodiments disclosed herein include, but are not limited to, those disclosed in U.S. Pat. Nos. 8,609,346; 8,577,106; 8,558,003; 8,492,107; 8,206,986; 7,993,868; 7,893,214; 7,858,803; 7,842,455; 7,815,894; 7,807,777; 7,794,948; 7,749,716; 7,611,910; 7,544,771; 7,015,044; 6,821,504; 6,485,911; 6,321,105; 6,024,707, and the like.

In some embodiments, the regenerative cells (e.g., adipose-derived regenerative cells, such as concentrated populations of adipose-derived cells comprising stem cells) and/or adipose-derived microparticles disclosed herein reduce or ameliorate physiological features of Alzheimer's (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing Alzheimer's.

In some embodiments, administration of the regenerative cells (e.g., adipose-derived regenerative cells, such as concentrated populations of adipose-derived cells comprising stem cells) and/or adipose-derived microparticles disclosed herein reduces or prevents the further accumulation of amyloid beta deposits, as discussed above. In some embodiments, administration of the regenerative cells and/or adipose-derived microparticles disclosed herein reduces or prevents the accumulation of neurofibrillary tangles and/or Tau protein variants using art-accepted methods. In some embodiments, administration of the regenerative cells and/or adipose-derived microparticles disclosed herein results in improvement in cognitive indices used in the assessment of Alzheimer's disease.

### Multiple Sclerosis (“MS”)

Multiple sclerosis (MS) is a chronic autoimmune inflammatory disease of the central nervous system. It is a common cause of persistent disability in young adults. In patients suffering from MS, the immune system destroys the myelin sheet of axons in the brain and the spinal cord, causing a variety of neurological pathologies. In the most common form of MS, Relapsing-Remitting, episodes of acute worsening of neurological function (exacerbations, attacks) are followed by partial or complete recovery periods (remissions) that are free of disease progression (stable). It has been reported that ninety percent of patients with multiple sclerosis initially present with a clinically isolated syndrome due to an inflammatory demyelinating lesion in the optic nerve, brain stem, or spinal cord. About thirty percent of those patients with a clinically isolated syndrome progress to clinically definite MS within 12 months after presentation. The subsequent progression of the disease can vary significantly from patient to patient. The progression can range from a benign course to a classic relapsing-remitting, chronic progressive, or rare fulminant course. Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction. (EMEA Guideline, 2006). Glutamate excitotoxicity and consequent neuroinflammation are central to the pathology and progression of multiple sclerosis.

According to some of the embodiments disclosed herein, provided are compositions and methods for the treatment or prevention of multiple sclerosis in a subject in need thereof. Many methods for the identification of subjects that have or that are at risk of developing multiple sclerosis, useful in the embodiments disclosed herein, are known in the art. Non-limiting examples of methods used to identify subject having or that are at risk of developing MS useful in the methods described herein include, but are not limited to, those disclosed in U.S. Pat. Nos. 8,048,639; 7,906,291; 7,572,592; 7,537,900; and 8,506,933; as well as in U.S. Patent Application Publication No's: 2013/0184173; 2013/01557270; 2011/0092383; 2010/0284933; 2010/0227337, 2006/0003327, and the like.

In some embodiments, the regenerative cells (e.g., adipose-derived regenerative cells, such as concentrated populations of adipose-derived cells comprising stem cells) and/or adipose-derived microparticles disclosed herein reduce or ameliorate physiological features of multiple sclerosis (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing multiple sclerosis.

In some embodiments, the regenerative cells (e.g., adipose-derived regenerative cells, such as concentrated populations of adipose-derived cells comprising stem cells) and/or adipose-derived microparticles disclosed herein can slow the progression of MS. Accordingly, some embodiments include assessing the progression of MS. Methods for tracking the progression of MS useful in the embodiments disclosed herein can include assessment and scoring of patients' function in attacks and accumulated disabilities during the attacks. Tools useful in the embodiments disclosed herein to assess the progression of MS include, but are not limited to the Expanded Disability Status Scale (EDSS) (see, e.g., Kurtzke, et al. (1983) Neurology 33 (11): 1444-1452); The multiple sclerosis functional composite measure (Fischer, et al., (1999) Multiple Sclerosis, 5 (4): 244-250), Poser, et al. (1983) Ann. Neurol. 13 (3): 227-230, as well as assessments using imaging techniques such as MRI, and the like (see, e.g., Barkhoff, et al. (1999), Multiple Sclerosis. 5(4):283-286).

### Huntington's Disease

Huntington disease, also called Huntington chorea, is invariably fatal, hereditary neurological disease that is characterized by irregular and involuntary movements of the muscles and progressive loss of cognitive ability. Symptoms of Huntington disease usually appear between the ages of 35 and 50 and worsen over time. They begin with occasional jerking or writhing movements, called choreiform movements, or what appear to be minor problems with coordination; these movements, which are absent during sleep, worsen over the next few years and progress to random, uncontrollable, and often violent twitchings and jerks. Symptoms of mental deterioration may appear including apathy, fatigue, irritability, restlessness, or moodiness; these symptoms may progress to memory loss, dementia, bipolar disorder, or schizophrenia.

Activated microglia are implicated in Huntington's disease, as they are prevalent in all grades of Huntington's pathology, accumulated with increasing grade, and grew in density in relation to the degree of neuronal loss. Excitotoxicity and neuroinflammation are also key features of disease pathology and progression, and it has been demonstrated that the function of GLT-1, a key glutamate receptor involved in clearance of extracellular glutamate in the CNS is impaired in Huntington's disease. In mouse models of Huntington's disease, brains have an increased extracellular glutamate concentration and a reduced expression level of GLT-1. See, e.g., Behrens (2002) Brain 125:190-1922.

Some of the embodiments disclosed herein provide compositions and methods for the treatment or prevention of Huntington's disease in a subject in need thereof. Many methods for the identification of subjects that have or that are at risk of developing Huntington's disease, useful in the embodiments disclosed herein, are known in the art. For example, diagnosis of Huntington's disease can include, for example, clinical history, neurologic and psychiatric examinations, neuroimaging, and genetic testing (including but not limited to tests that count the number of CAG repeats in each of the two HTT alleles). Various other methods of identifying subjects that have or that are at risk of developing Huntington's disease useful in the embodiments disclosed herein include, but are not limited to, those described in International Patent Application Publications WO 06/061610, WO 02/029408, WO 02008449; U.S. Pat. No. 8,506,957, and the like.

In some embodiments, the regenerative cells (e.g., adipose-derived regenerative cells, such as concentrated populations of adipose-derived cells comprising stem cells) and/or adipose-derived microparticles disclosed herein reduce or ameliorate physiological features of Huntington's disease (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing Huntington's disease.

In some embodiments, following administration of the regenerative cells (e.g., adipose-derived regenerative cells, such as concentrated populations of adipose-derived cells comprising stem cells) and/or adipose-derived microparticles as disclosed herein, the subject will show improvement in one or more art-known tests to assess the progression of Huntington's disease including, but not limited to, the Total Functional Capacity (TFC) portion of the Unified Huntington's Disease Rating Scale (UHDRS) (see, e.g., Kieburtz, et al. (1996) Mov Dis. 11:136-142), the mini-mental state examination (MMSE)(see, e.g., Folstein, et al. (1975) J. Psych. Res. 12 (3): 189-98, and the like. In some embodiments, subsequent to administration of the regenerative cells (e.g., adipose-derived regenerative cells, such as concentrated populations of adipose-derived cells comprising stem cells) disclosed herein, the subject exhibits improvement in one or more of the motor skill abnormalities associated with Huntington's disease including, of restlessness, abnormal eye movements, hyperreflexia, impaired finger tapping, rapid alternating hand movements and mild dysarthria (speech); involuntary motor abnormalities such as chorea (rapid, ceaseless movements) bradykinesia, hypokinesia, rigidity and dystonia; voluntary motor impairments such as dysphagia (swallowing), dysarthria, and gait disturbances. In some embodiments, subsequent to administration of the regenerative cells (e.g., adipose-derived regenerative cells, such as concentrated populations of adipose-derived cells comprising stem cells) and/or adipose-derived microparticles as disclosed herein, the subject exhibits improvement in one or more of muscle wasting, dehydration, and weight loss; non-motor symptoms of cognitive deficits in concentration, organization, spatial perception, memory skills (dementia), and non-cognitive psychiatric deficits of depression (low energy, sleep disturbances), personality changes (irritability, low energy, apathy anhedonia) and bipolar disorder (delusions, hallucinations, paranoia) associated with Huntington's disease.

### Epilepsy

Epilepsy is a neurological disorder in which normal brain function is disrupted as a consequence of intensive burst activity from groups of neurons. See, Wyllie, E., “The Treatment of Epilepsy Principles and Practice” Lippincot, Williams, and Wilkins, New York (2001)). Epilepsies result from long-lasting plastic changes in the brain affecting the expression of receptors and channels, and involve sprouting and reorganization of synapses, as well as reactive gliosis. Heinemann et al., (1999) “Adv. Neurol. 79:583-590 (1999); Rogawski et al., (2004) Nat. Rev. Neurosci. 5:553-564.

Studies have demonstrated a key role of glutamate, active neuroinflammation, blood brain barrier disruption, decreased cerebral blood flow, and marked cellular injury in pediatric epilepsy and epilepsy of diverse etiologies. See, e.g., Choi, et al. (2009) J. Neuroinflamm. 6:30; Theoharides (2011) J. Neuroinflamm. 8:168. For example, activation of microglia is also observed in epileptic subjects. See, e.g., Sheng et al., (1994) J. Neurochem. 63:1872.

Some of the embodiments disclosed herein provide compositions and methods for the treatment or prevention of epilepsy in a subject in need thereof. Many methods for the identification of subjects that have or that are at risk of developing epilepsy, useful in the embodiments disclosed herein, are known in the art. Besides the manifestation of observable symptoms (e.g., including convulsions, muscle spasms, loss of consciousness, etc.), epilepsy can be detected and/or diagnosed by the use of various procedures. These can include electroencephalographyy (EEG), video EEG, computerized tomography (CT) scans, magnetic resonance imaging (MRI), positron emission tomography (PET), and/or single-photon emission computer tomography (SPECT). Other methods of identifying subjects that have, or that are at risk of developing, epilepsy include, but not limited to, those disclosed in International Patent Application Publication No's WO 1999/058968, WO 2011/041433; European Patent Application Publication No. EP 1904630A1, Canadian Patent Application Publication No. CA2443550. The skilled artisan will readily appreciate that many other art-accepted methods of identifying subjects that have or that are at risk of developing epilepsy are known and useful in the embodiments disclosed herein.

In some embodiments, the regenerative cells (e.g., adipose-derived regenerative cells, such as concentrated populations of adipose-derived cells comprising stem cells) and/or adipose-derived microparticles disclosed herein reduce or ameliorate physiological features of epilepsy (e.g., reduce or ameliorate one more aspects of neuroexcitotoxcity and neuroinflammation, BBB alterations, cerebral metabolism and blood flow, and the like, discussed herein below). The compositions and methods disclosed herein are useful in modulating or reducing microglial activation, increasing the ratio of M2:M1 activated microglial cells, increasing the loco-regional concentration of M2 macrophages in the CNS, reduce oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing epilepsy.

In some embodiments, following administration of the regenerative cells (e.g., adipose-derived regenerative cells, such as concentrated populations of adipose-derived cells comprising stem cells) and/or adipose-derived microparticles disclosed herein, the subject will show improvement, e.g., in the severity and or the frequency of epileptic events or symptoms of epilepsy as described above.

### Spinal Cord Injury (“SCI”)

Spinal cord injury Spinal cord injury (SCI) resulting in severe and permanent neurologic dysfunction is a serious, though relatively rare, event with enormous impact on the quality of life of the patient. While annual incidence is low (between 20 to 50 cases per million) it is estimated that worldwide there are more than two million people with severe disability from SCI. See, e.g., Fawcett, et al. (2007) Spinal Cord 45(3):190-205. There is currently no successful treatment for this dysfunction.

As with traumatic brain injury, spinal cord injury occurs in two phases (1) initial acute trauma and (2) progressive secondary injury caused by the sequelae of trauma. Traumatic SCI causes mechanical injury and death of local neuronal and vascular tissues. This initial damage is then exacerbated by secondary effects including glutamate excitotoxicity, ischemia, edema, and inflammation that lead to additional cell death. Later phases of SCI are associated with ongoing apoptosis, activation of astrocyte proliferation leading to formation of a glial scar, Wallerian degeneration, and, ultimately, formation of cysts, Schwannoses, and replacement of the glial scar with a fibrotic scar. In the face of this progressive injury, spontaneous regenerative mechanisms such as angiogenesis, axonal sprouting, expression of neuronal growth-associated genes, and collateral sprouting of uninjured axons are inadequate to elicit meaningful recovery of neurologic function.

The term “spinal cord injury” as used herein encompasses any form of physical, chemical or genetic trauma to the spinal cord. Exemplary physical trauma can be a tissue insult such as an abrasion, incision, contusion, puncture, compression etc., such as an insult arising from traumatic contact of a foreign object with any locus of or appurtenant/adjacent to the head, neck or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor or from disease (poliomyelitis, spina bifida, Friedreich's Ataxia, etc.). Spinal cord injury can also caused be caused by compression by bone fragments or disc material.

Some of the embodiments disclosed herein provide compositions and methods for the treatment or amelioration of SCI. Several methods for the identification of subjects that have or that are at risk of developing SCI, useful in the embodiments disclosed herein, are known in the art. For example, subjects at risk of developing SCI include, for example subjects that have a genetic predisposition to spinal cord injury (e.g., in the case of spinal deformity or achondroplasia). Likewise, art-recognized methods for diagnosing SCI are well-known and include, but are not limited to CT scans or MRI scans of the spinal cord, myelograms, somatosensory evoked potential (SSEP) testing or magnetic stimulation, X-rays and the like.

In some embodiments, the regenerative cells (e.g., adipose-derived regenerative cells, such as concentrated populations of adipose-derived cells comprising stem cells) and/or adipose-derived microparticles as disclosed herein reduce or ameliorate physiological features of SCI (e.g., reduce or ameliorate one more aspects of neuroexcitotoxcity and neuroinflammation and the like, discussed herein). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibiting neuronal apoptosis, preventing or inhibiting dendritic and synaptic loss, reducing the number of calcium-permeable AMPA receptors in subjects identified as having or at risk of developing SCI.

In some embodiments, the regenerative cells (e.g., adipose-derived regenerative cells, such as concentrated populations of adipose-derived cells comprising stem cells) and/or adipose-derived microparticles disclosed herein effectuate improvement in one or more symptoms of, or a reduction in the progression of one or more symptoms of, said spinal cord injury as detected by sensory testing. Sensory testing can be performed at the following levels:

C2—Occipital protuberance

C3—Supraclavicular fossa

C4—Top of the acromioclavicular joint

C5—Lateral side of antecubital fossa

C6—Thumb

C7—Middle finger

C8—Little finger

T1—Medial side of antecubital fossa

T2—Apex of axilla

T3—Third intercostal space (IS)

T4—Fourth IS at nipple line

15—Fifth IS (midway between T4 and T6)

T6—Sixth IS at the level of the xiphisternum

T7—Seventh IS (midway between T6 and T8)

T8—Eighth IS (midway between T6 and T10)

T9—Ninth IS (midway between T8 and T10)

T10—10th IS or umbilicus

Tl 1—1 lth IS (midway between T10 and T12)

T12—Midpoint of inguinal ligament

LI—Half the distance between T12 and L2

L2—Midanterior thigh

L3—Medial femoral condyle

L4—Medial malleolus

L5—Dorsum of the foot at third metatarsophalangeal joint

SI—Lateral heel

S2—Popliteal fossa in the midline S3—Ischial tuberosity

S4-5—Perianal area (taken as 1 level)

Sensory scoring is for light touch and pinprick, as follows:

0—Absent

1—Impaired or hyperesthesia

2—Intact

A score of zero is given if the patient cannot differentiate between the point of a sharp pin and the dull edge.

Accordingly, following administration of a composition comprising regenerative cells (e.g., adipose-derived regenerative cells, such as concentrated populations of adipose-derived cells comprising stem cells) and/or adipose-derived microparticles in accordance with the embodiments disclosed herein to a subject with SCI, the subject exhibits a one or two point increase in sensory scoring corresponding to one or more of C2, C3, C4, C5, C6, C7, C8, T1, T2, T3, T4, T5, T6, T7, T8, T9, T10, Ti 1, T12, LI, L2, L3, L4, L5, S1, S2, S3, S4 and S5.

In some embodiments subjects with SCI that are administered compositions comprising regenerative cells and/or adipose-derived microparticles according to the embodiments disclosed herein exhibit improvement in the Functional Independence Measure (FIM). The FIM focuses on six areas of functioning: self-care, sphincter control, mobility, locomotion, communication and social cognition. Within each area, two or more specific activities/items are evaluated, with a total of 18 items. For example, six activity items (eating, grooming, bathing, dressing-upper body, dressing-lower body, and toileting) comprise the self-care area. Each of the 18 items is evaluated in terms of independence of functioning, using a seven-point scale:

Independent (no human assistance is required):

7=Complete independence: The activity is typically performed safely, without modification, assistive devices or aids, and within reasonable time.

6=Modified independence: The activity requires an assistive device and/or more than reasonable time and/or is not performed safely.

Dependent (human supervision or physical assistance is required):

5=Supervision or setup: No physical assistance is needed, but cuing, coaxing or setup is required.

4=Minimal contact assistance: Subject requires no more than touching and expends 75% or more of the effort required in the activity. 3=Moderate assistance: Subject requires more than touching and expends 50±75% of the effort required in the activity.

2=Maximal assistance: Subject expends 25±50% of the effort required in the activity. 1=Total assistance: Subject expends 0±25% of the effort required in the activity.

Thus, the FIM total score (summed across all items) estimates the cost of disability in terms of safety issues and of dependence on others and on technological devices. The profile of area scores and item scores pinpoints the specific aspects of daily living that have been most affected by SCI. In some embodiments following administration of the adipose-derived cells as disclosed herein to a subject with SCI according to the embodiments disclosed herein, the subject exhibits a one, two, three, four, five or six point increase in functioning of the patient according to the FIM scale.

### PTSD

The embodiments disclosed herein include identification of individuals that have or that are at risk of developing post-traumatic stress syndrome. Any art-recognized method can be used in the embodiments disclosed herein to identify subject that have or that are at risk of developing PTSD.

By way of example only, a subject can be diagnosed as having PTSD using the criteria set forth in “The Diagnostic and Statistical Manual of Mental Disorders-IV-Text revised (DSM-IV-TR), a handbook for mental health professionals that lists categories of mental disorders and the criteria, classifies post-traumatic stress disorder as an anxiety disorder. According to the DSM-IV-TR, a PTSD diagnosis can be made if:

1. the subject experienced, witnessed, or was confronted with an event or events that involved actual or threatened death or serious injury, or a threat to the physical integrity of self or others and the response involved intense fear, helplessness, or horror;

2. as a consequence of the traumatic event, the subject experiences at least 1 re-experiencing/intrusion symptom, 3 avoidance/numbing symptoms, and 2 hyperarousal symptoms, and the duration of the symptoms is for more than 1 month; and

3. the symptoms cause clinically significant distress or impairment in social, occupational, or other important areas of functioning.

In certain embodiments, a scale is used to measure a sign, symptom, or symptom cluster of posttraumatic stress disorder, and post-traumatic stress disorder is diagnosed on the basis of the measurement using that scale. In certain embodiments, a “score” on a scale is used to diagnose or assess a sign, symptom, or symptom cluster of post-traumatic stress disorder. In certain embodiments, a “score” can measure at least one of the frequency, intensity, or severity of a sign, symptom, or symptom cluster of post-traumatic stress disorder.

As used herein, the term “scale” refers to a method to measure at least one sign, symptom, or symptom cluster of post-traumatic stress disorder in a patient. In certain embodiments, a scale may be an interview or a questionnaire. Non-limiting examples of scales useful in the embodiments disclosed herein include, but are not limited to the Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

As used herein, the terms “sign” and “signs” refer to objective findings of a disorder. In certain embodiments, a sign can be a physiological manifestation or reaction of a disorder. In certain embodiments, a sign may refer to heart rate and rhythm, body temperature, pattern and rate of respiration, blood pressure. In certain embodiments, signs can be associated with symptoms. In certain embodiments, signs can be indicative of symptoms.

In some embodiments, if the subject exhibits one or more signs, symptoms, or symptom clusters of post-traumatic stress disorder, e.g., as discussed herein, the subject is diagnosed with post-traumatic stress disorder. As used herein, the term “symptom” and “symptoms” refer to subjective indications that characterize a disorder. Symptoms of post-traumatic stress disorder include, but not limited to recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In certain embodiments, potentially threatening stimuli can cause hyperarousal or anxiety. In certain embodiments, the physiological reactivity manifests in at least one of abnormal respiration, abnormal cardiac rate of rhythm, abnormal blood pressure, abnormal function of a special sense, and abnormal function of sensory organ. In certain embodiments, restricted range of effect characterized by diminished or restricted range or intensity of feelings or display of feelings can occur and a sense of a foreshortened future can manifest in thinking that one will not have a career, marriage, children, or a normal life span. In certain embodiments, children and adolescents may have symptoms of post-traumatic stress disorder such as, for example and without limitation, disorganized or agitated behavior, repetitive play that expresses aspects of the trauma, frightening dreams which lack recognizable content, and trauma-specific reenactment.

As used herein, the term “symptom cluster” refers to a set of signs, symptoms, or a set of signs and symptoms, that are grouped together because of their relationship to each other or their simultaneous occurrence. For example, in certain embodiments posttraumatic stress disorder is characterized by three symptom clusters: re-experiencing/intrusion, avoidance/numbing, and hyperarousal.

In some embodiments, diagnosis of subjects that have or that are at risk of developing PTSD can be made using any art-recognized PTSD biomarker. Non-limiting examples of biomarkers useful in the identification of subjects that have or that are at risk of developing PTSD include, but are not limited to, those described in International Patent Application Publication No's. WO 2013/0400502, WO 2012/06407, WO 2013/181472, European Patent No. EP 2334816, and the like.

In some embodiments, the regenerative cells (e.g., adipose-derived regenerative cells such as concentrated populations of adipose-derived cells comprising stem cells) and/or adipose-derived microparticles disclosed herein reduce or ameliorate physiological and/or psychiatric and/or psychological signs or symptoms of PTSD (e.g., reduce or ameliorate one more aspects of neuroexcitotoxcity and neuroinflammation, BBB alterations, cerebral metabolism and blood flow, and the like, discussed herein). The compositions and methods disclosed herein are useful in modulating or reducing microglial activation, increasing the ratio of M2:M1 activated microglial cells, increasing the loco-regional concentration of M2 macrophages in the CNS, reducing oxidative damage, reducing reactive oxygen species and/or reactive nitrogen species in the CNS, reducing lipid peroxidation, preventing or inhibit neuronal apoptosis, prevent or inhibit dendritic and synaptic loss, reducing the number of calcium-permeable AMPA receptors, enhancing BBB integrity, increasing pericyte coverage in the BBB, enhancing cerebral blood flow and improve cerebral metabolism in subjects identified as having or at risk of developing PTSD.

### Methods of Obtaining Regenerative Cells and Adipose-Derived Microparticles

As mentioned above, a population of “regenerative cells” disclosed herein can be a homogeneous or heterogeneous population of cells that cells that which cause or contribute to complete or partial regeneration, restoration, or substitution of structure or function of an organ, tissue, or physiologic unit or system to thereby provide a therapeutic, structural or cosmetic benefit. Examples of regenerative cells include, but are not limited to adult stem cells, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

The regenerative cells disclosed herein can be isolated from various tissues, including, but not limited to bone marrow, placenta, adipose tissue, skin, eschar tissue, endometrial tissue, adult muscle, corneal stroma, dental pulp, Wharton's jelly, amniotic fluid, and umbilical cord. The regenerative cells disclosed herein can be isolated from the tissues above using any means known to those skilled in the art or discovered in the future.

By way of example only, regenerative cells can be isolated from adipose tissue by a process wherein tissue is excised or aspirated. Excised or aspirated tissue can be washed, and then enzymatically or mechanically disaggregated in order to release cells bound in the adipose tissue matrix. Once released, the cells can be suspended. By way of example only, regenerative cells useful in the embodiments disclosed herein can be isolated using the methods and/or devices described in U.S. Pat. Nos. 7,390,484; 7,585,670, 7,687,059, 8,309,342, 8,440,440. US Patent Application Publication No's. 2013/0164731, 2013/0012921, 2012/0164113, US2008/0014181. International Patent Application Publication No. WO2009/073724, WO/2013030761 and the like, each of which is herein incorporated by reference.

Exemplary, non-limiting methods for isolation of regenerative cells from bone marrow useful in the embodiments disclosed herein are described in U.S. Pat. No. 5,879,940, U.S. Patent Application Publication No's 2013/0101561, 2013/0266541 European Patent Application Publication No. EP2488632A1, EP0241578A2, EP2624845A2, International Patent Application Publication No. WO2011047289A1, WO1996038482A, each of which is herein incorporated by reference.

Exemplary, non-limiting methods for isolation of regenerative cells from placental tissue useful in the embodiments disclosed herein are described in U.S. Pat. No. 8,580,563, U.S. Patent Application Publication No. 20130040281, International Patent Application Publication No. WO2003089619A, Klein, et al. (2011) Methods Mol Biol. 698:75-88, Vellasamy, et al. (2012) World J Stem Cells 4(6): 53-61; Timmins, et al. (2012) Biotechnol Bioeng. 109(7):1817-26; Semenov, et al. (2010) Am J Obstet Gynecol 202:193-e.13, and the like, each of which is herein incorporated by reference.

Exemplary, non-limiting methods for isolation of regenerative cells from skin useful in the embodiments disclosed herein are described in Toma, et al. (2001), Nat Cell Biol. 3(9):778-84; Nowak, et al. (2009), Methods Mol Biol. 482:215-32; U.S. Patent Application Publication No. 2007/0248574, and the like, each of which is herein incorporated by reference.

Exemplary, non-limiting methods for isolation of regenerative cells from eschar tissue useful in the embodiments disclosed herein are described in Van der Veen, et al. (2012), Cell Transplant. 21(5):933-42, and elsewhere herein below.

Exemplary, non-limiting methods for isolation of regenerative cells from endometrial tissue useful in the embodiments disclosed herein are described in U.S. Patent Application Publication No. 2013/0156726, 2008/0241113, and the like, each of which is herein incorporated by reference in its entirety.

Exemplary, non-limiting methods for isolation of regenerative cells from muscle tissue useful in the embodiments disclosed herein are described in U.S. Pat. No. 6,337,384, U.S. Patent Application Publication No. 2001/019966, 2011/0033428, 2005/0220775, and the like, each of which is herein incorporated by reference.

Exemplary, non-limiting methods for isolation of regenerative cells from corneal tissue useful in the embodiments disclosed herein are described in U.S. Patent Application Publication No. 2005084119, Sharifi, et al. (2010) Biocell. 34(1):53-5, and the like, each of which is herein incorporated by reference.

Exemplary, non-limiting methods for isolation of regenerative cells from dental pulp useful in the embodiments disclosed herein are described in U.S. Patent Application Publication No. 2012/0251504, Gronthos, et al. (2011) Methods Mol Biol. 698:107-21; Suchánek, et al. Acta Medica (Hradec Kralove). 2007; 50(3):195-201; Yildirm, Sibel, “Isolation Methods of Dental Pulp Stem Cells,” in Dental Pulp Stem Cells: Springer Briefs in Stem Cells, pp. 41-51, © 2013, Springer New York, New York, N.Y., and the like, each of which is herein incorporated by reference.

Exemplary, non-limiting methods for isolation of regenerative cells from Wharton's jelly useful in the embodiments disclosed herein are described in U.S. Patent Application Publication No's. 2013/0183273, 2011/0151556, International Patent Application Publication No. WO 04/072273A1, Sheshareddy, et al. (2008) Methods Cell Biol. 86:101-19, Mennan, et al. (2013) BioMed Research International, Article ID 916136, Corotchi, et al. (2013) Stem Cell Research & Therapy 4:81, and the like, each of which is herein incorporated by reference.

Exemplary, non-limiting methods for isolation of regenerative cells from amniotic fluid useful in the embodiments described herein are described in U.S. Pat. No. 8,021,876, International Patent Application Publication No. WO 2010/033969A1, WO 2012/014247A1, WO 2009/052132, U.S. Patent Application Publication No. 2013/0230924, 2005/0054093, and the like, each of which is herein incorporated by reference.

Exemplary, non-limiting methods for isolation of regenerative cells from the umbilical cord useful in the embodiments described herein are described in U.S. Patent Application Publication No. 20130065302, Reddy, et al. (2007), Methods Mol Biol. 407:149-63, Hussain, et al. (2012) Cell Biol Int 36(7):595-600, Pham, et al. (2014) Journal of Translational Medicine 2014, 12:56, Lee, et al. (2004) Blood 103(5): 1669-1675, and the like, each of which is herein incorporated by reference.

The regenerative cells in the methods and compositions described herein can be a heterogeneous population of cells that includes stem and other regenerative cells. In some embodiments, the regenerative cells in the methods and compositions described herein can include stem and endothelial precursor cells. In some embodiments, the regenerative cells can include stem and pericyte cells. In some embodiments, the regenerative cells can include stem cells and leukocytes. For example, in some embodiments, the regenerative cells can include stem cells and macrophages. In some embodiments, the regenerative cells can include stem cells and M2 macrophages. In some embodiments, the regenerative cells can include pericytes and endothelial precursor cells. In some embodiments, the regenerative cells can include platelets. Preferably, the regenerative cells comprise stem cells and endothelial precursor cells. In some embodiments, the regenerative cells can include regulatory cells such as Treg cells.

In some embodiments, the regenerative cells are not cultured prior to use. By way of example, in some embodiments, the regenerative cells are for use following isolation from the tissue of origin, e.g., bone marrow, placenta, adipose tissue, skin, eschar tissue, endometrial tissue, adult muscle, cornea stroma, dental pulp, Wharton's jelly, amniotic fluid, umbilical cord, and the like.

In some embodiments, the regenerative cells are cultured prior to use. For example, in some embodiments, the regenerative cells are subjected to “limited culture,” i.e., to separate cells that adhere to plastic from cells that do not adhere to plastic. Accordingly, in some embodiments, the regenerative cells are “adherent” regenerative cells. An exemplary, non-limiting method of isolating adherent regenerative cells from adipose tissue are described e.g., in Zuk, et al. (2001). Exemplary, non-limiting method of isolating adherent regenerative cells from bone marrow are described, e.g., Pereira (1995) Proc. Nat. Acad. Sci. USA 92:4857-4861, Castro-Malaspina et al. (1980), Blood 56:289-30125; Piersma et al. (1985) Exp. Hematol. 13:237-243; Simmons et al., 1991, Blood 78:55-62; Beresford et al., 1992, J. Cell. Sci. 102:341-3 51; Liesveld et al. (1989) Blood 73:1794-1800; Liesveld et al., Exp. Hematol 19:63-70; Bennett et al. (1991) J. Cell. Sci. 99:131-139), U.S. Pat. No. 7,056,738, and the like.

In some embodiments, the regenerative cells are cultured for more than 3 passages in vitro. For example, in some embodiments, the regenerative cells are cultured for 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more passages in vitro.

The regenerative cells described herein can be cultured according to approaches known in the art, and the cultured cells can be used in several of the embodied methods. For example, regenerative cells can be cultured on collagen-coated dishes or 3D collagen gel cultures in endothelial cell basal medium in the presence of low or high fetal bovine serum or similar product, as described in Ng, et al., (2004), Microvasc. Res. 68(3):258-64, incorporated herein by reference. Alternatively, regenerative cells can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture regenerative cells, as well.

Examples of basal culture medium that can be used to culture regenerative cells in vitro include, but are not limited to, EGM, RPMI, M199, MCDB131, DMEM, EMEM, McCoy's 5A, Iscove's medium, modified Iscove's medium, or any other medium known in the art to support the growth of blood endothelial cells. In some embodiments, the regenerative cells are cultured in EGM-2MV media. Examples of supplemental factors or compounds that can be added to the basal culture medium that could be used to culture regenerative cells include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyril cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, matrix metalloproteinases (such as MMP2 and MMP9), or any other compound known in the art to promote survival, proliferation or differentiation of endothelial cells.

Further processing of the cells may also include: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cell lines (including hybridoma cells); culture of cells for permeability studies; cells for RNAi and viral resistance studies; cells for knock-out and transgenic animal studies; affinity purification studies; structural biology applications; assay development and protein engineering applications.

In some embodiments, methods for isolating regenerative useful in the embodiments described herein can include positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

By way of example, the regenerative cells useful in the methods of treatment disclosed herein may be identified by different combinations of cellular and genetic markers. For example, in some embodiments, the regenerative cells express CD90. In some embodiments, the regenerative cells do not express significant levels of lin. In some embodiments, the regenerative cells do not express significant levels of ckit. In some embodiments, the regenerative cells are CD90+/lin−/ckit−/CD45−.

In some embodiments, the regenerative cells express STRO-1. In some embodiments, the regenerative cells express STRO-1 and CD49d. In some embodiments, the regenerative cells express STRO-1, CD49d, and one or more of CD29, CD44, CD71, CD90, C105/SH2 and SH3. In some embodiments, the regenerative cells express STRO-1, CD49d, and one or more of CD29, CD44, CD71, CD90, C105/SH2 and SH3, but express low or undetectable levels of CD106.

In some embodiments, the regenerative cells express one or more of STRO-1, CD49d, CD13, CD29, SH3, CD44, CD71, CD90, and CD105, or any combination thereof. By way of example only, in some embodiments, the regenerative cells express each of do not express significant levels of CD31, CD34, CD45 and CD104 and do not express detectable levels of CD4, CD8, CD11, CD14, CD16, CD19, CD33, CD56, CD62E, CD106 and CD58.

In some approaches, the regenerative cells are CD14 positive and/or CD11b positive.

In some embodiments, the cells are depleted for cells expressing the markers CD45(+). In some embodiments, the cells are depleted for cells expressing glycophorin-A (GlyA). In some embodiments, the cells are depleted for CD45(+) and GlyA(+) cells.

Negative selection of cells, e.g., depletion of certain cell types from a heterogeneous population of cells can done using art-accepted techniques, e.g., utilizing micromagnetic beads or the like. In some embodiments, the regenerative cells are CD34+.

In some embodiments, the regenerative cells are not cryopreserved. In some embodiments, the regenerative cells are cryopreserved. For example, in some embodiments, the regenerative cells include cryopreserved cells, e.g., as described in Liu, et al. (2010) Biotechnol Prog. 26(6):1635-43, Carvalho, et al. (2008) Transplant Proc.; 40(3):839-41, International Patent Application Publication No. WO 97/039104, WO 03/024215, WO 2011/064733, WO 2013/020492, WO 2008/09063, WO 2001/011011, European Patent No. EP0343217 B1, and the like.

In some embodiments, the regenerative cells are adipose-derived regenerative cells. The adipose-derived cells used in the embodiments described herein, (e.g., adipose-derived cell populations comprising stem cells, adipose-derived cell populations comprising regenerative cells, adipose-derived cell populations comprising stem cells and other regenerative cells, adipose-derived cell populations comprising stem cells and precursor cells) can be obtained by methods known in the art, e.g., for the preparation of the stromal vascular fraction. In some embodiments, adipose tissue is processed to obtain a refined, enriched, concentrated, isolated, or purified population of adipose-derived cells, e.g., a population of adipose-derived cells comprising stem cells (e.g., present at a frequency of more than 0.1%, more than 1%, more than 2%, of the cellular component), a population of adipose-derived cells comprising regenerative cells, a population of adipose-derived cells comprising stem and other regenerative cells, and the like useful in the embodiments disclosed herein, using a cell processing unit, gradient sedimentation, filtration, or a combination of any one or more of these approaches. In general, adipose tissue is first removed from a subject (e.g., a mammal, a domestic animal, a rodent, a horse, a dog, cat, or human) then it is processed to obtain a cell population, e.g., a population of adipose-derived cells comprising stem cells, a population of adipose-derived cells comprising regenerative cells, a population of adipose-derived cells comprising stem and other regenerative cells, and the like.

In some embodiments, the adipose-derived cells are obtained from an autologous donor (i.e., the same subject to whom the cells are administered). In some embodiments, the adipose-derived cells are obtained from a non-autologous donor (e.g., an allogeneic or xenogenic donor). In embodiments wherein the donor is allogeneic, an appropriate donor can be selected using methods known in the art, for example, methods used for selection of bone marrow donors.

In some embodiments, adipose-derived regenerative microparticles are obtained from an autologous donor (i.e., the same subject to whom the cells are administered). In some embodiments, the adipose-derived micro-particles are obtained from a non-autologous donor (e.g., an allogenic or xenogenic donor). Advantageously, administration of adipose-derived micro-particles without cells reduces the risk of adverse reactions in non-autologous subjects.

In some embodiments, adipose-derived cells (e.g., adipose-derived regenerative cells, such as concentrated populations of adipose-derived cells comprising stem cells) and/or microparticles are isolated from adipose tissue in system that maintains a closed, sterile fluid/tissue pathway. Accordingly, in some embodiments, the adipose-derived cells (e.g., adipose-derived regenerative cells, such as concentrated populations of adipose-derived cells comprising stem cells) and/or microparticles are isolated from adipose tissue using a process and/or device that does not expose the cells to the external environment during isolation and/or administration.

The volume of adipose tissue collected from the patient can vary from about 1 cc to about 2000 cc and in some embodiments up to about 3000 cc. The volume of tissue removed will vary from patient to patient and will depend on a number of factors including but not limited to: age, body habitus, coagulation profile, hemodynamic stability, severity of insufficiency or injury, co-morbidities, and physician preference.

Adipose tissue can be obtained by any method known to a person of ordinary skill in the art. For example, the adipose tissue may be removed from a subject by suction-assisted lipoplasty, ultrasound-assisted lipoplasty, or excisional lipectomy. In addition, the procedures may include a combination of such procedures, such as a combination of excisional lipectomy and suction-assisted lipoplasty. If the tissue or some fraction thereof is intended for re-implantation into a subject, the adipose tissue should be collected in a manner that preserves the viability of the cellular component and that minimizes the likelihood of contamination of the tissue with potentially infectious organisms, such as bacteria and/or viruses. Thus, the tissue extraction should be performed in a sterile or aseptic manner to minimize contamination. Suction-assisted lipoplasty may be desired to remove the adipose tissue from a patient as it provides a minimally invasive method of collecting tissue with minimal potential for stem cell damage that may be associated with other techniques, such as ultrasound-assisted lipoplasty.

Accordingly, adipose tissue provides a rich source of a population of cells that is easily enriched for adipose-derived stem cells, adipose-derived regenerative cells (e.g., one or more of adipose-derived stem cells, precursor cells, progenitor cells and the like), adipose-derived stem and other regenerative cells, and microparticles. Collection of adipose tissue is also more patient-friendly and is associated with lower morbidity than collection of a similar volume of, for example, skin or a much larger volume of tonsil.

For suction-assisted lipoplastic procedures, adipose tissue is collected by insertion of a cannula into or near an adipose tissue depot present in the patient followed by aspiration of the adipose into a suction device. In some embodiments, a small cannula may be coupled to a syringe, and the adipose tissue may be aspirated using manual force. Using a syringe or other similar device may be desirable to harvest relatively moderate amounts of adipose tissue (e.g., from 0.1 ml to several hundred milliliters of adipose tissue). Procedures employing these relatively small devices require only local anesthesia. Larger volumes of adipose tissue (e.g., greater than several hundred milliliters) may require general anesthesia at the discretion of the donor and the person performing the collection procedure. When larger volumes of adipose tissue are to be removed, relatively larger cannulas and automated suction devices may be employed.

Excisional lipectomy procedures include, and are not limited to, procedures in which adipose tissue-containing tissues (e.g., skin) is removed as an incidental part of the procedure; that is, where the primary purpose of the surgery is the removal of tissue (e.g., skin in bariatric or cosmetic surgery) and in which adipose tissue is removed along with the tissue of primary interest. Subcutaneous adipose tissue may also be extracted by excisional lipectomy in which the adipose tissue is excised from the subcutaneous space without concomitant removal of skin.

The amount of tissue collected can depend on a number of variables including, but not limited to, the body mass index of the donor, the availability of accessible adipose tissue harvest sites, concomitant and pre-existing medications and conditions (such as anticoagulant therapy), and the clinical purpose for which the tissue is being collected. Experience with transplant of hematopoietic stem cells (bone marrow or umbilical cord blood-derived stem cells used to regenerate the recipient's blood cell-forming capacity) shows that engraftment is cell dose-dependent with threshold effects (Smith, et al., 1995; Barker, et al., 2001, both incorporated herein by reference in their entirety). Thus, it is possible that the general principle that “more is better” will be applied within the limits set by other variables and that where feasible the harvest will collect as much tissue as possible.

The adipose tissue that is removed from a patient is then collected into a device (e.g., cell processing unit, centrifuge, or filtration unit) for further processing so as to remove collagen, adipocytes, blood, and saline, thereby obtaining a cell population comprising adipose derived cells, e.g., adipose-derived cells comprising stem cells, adipose-derived cells comprising regenerative cells, adipose-derived cells comprising stem and other regenerative cells, and the like, as well as adipose-derived microparticles. Preferably the population of adipose derived cells containing ADRCs (and optionally adipose-derived microparticles associated therewith) is free from contaminating collagen, adipocytes, blood, and saline. The major contaminating cells in adipose tissue (adipocytes) have low density and are easily removed by flotation.

Adipose tissue processing to obtain a refined, concentrated, and isolated population of adipose-derived cells, e.g., a population of adipose-derived cells comprising at least 0.1% stem cells, a population of adipose-derived cells comprising regenerative cells (e.g., one or more of stem cells, precursor cells, progenitor cells, the like), a population of adipose-derived cells comprising stem cells and other regenerative cells and the like, and modifications thereto are preferably performed using methods described, for example, in U.S. application Ser. No. 10/316,127 (U.S. Pat. App. Pub. No. 2003/0161816), entitled SYSTEMS AND METHODS FOR TREATING PATIENTS WITH PROCESSED LIPOASPIRATE CELLS, filed Dec. 9, 2002, and U.S. application Ser. No. 10/877,822 (U.S. Pat. App. Pub. No. 2005/0084961), entitled SYSTEMS AND METHODS FOR SEPARATING AND CONCENTRATING REGENERATIVE CELLS FROM TISSUE, filed Jun. 25, 2004; U.S. application Ser. No. 10/242,094, entitled PRESERVATION OF NON EMBRYONIC CELLS FROM NON HEMATOPOIETIC TISSUES, filed Sep. 12, 2002, which claims the benefit of U.S. App. Ser. No. 60/322,070 filed Sep. 14, 2001; U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004; all of which are hereby expressly incorporated by reference in their entireties. The applications above disclose the processing of adipose-derived cells in a system that is configured to maintain a closed, sterile fluid/tissue pathway. This can be achieved by use of a pre-assembled, linked set of closed, sterile containers and tubing allowing for transfer of tissue and fluid elements within a closed pathway. This processing set can be linked to a series of processing reagents (e.g., saline, enzymes, etc.) inserted into a device, which can control the addition of reagents, temperature, and timing of processing thus relieving operators of the need to manually manage the process. In a preferred embodiment, the entire procedure from tissue extraction through processing and placement into the recipient is performed in the same facility, indeed, even within the same room, of the patient undergoing the procedure. In preferred embodiments, the entire procedure from tissue extraction through processing is performed in a system that maintains a closed, sterile fluid/tissue pathway.

For many applications, preparation of the adipose-derived cell population, including the active cell population, e.g., the adipose-derived cells (such as adipose-derived regenerative cells, including cell populations wherein the frequency of stem cells is at least 0.1% of the cellular component) and/or adipose-derived microparticles requires depletion of the mature fat-laden adipocyte component of adipose tissue. This can be achieved by a series of washing and disaggregation steps in which the tissue is first rinsed to reduce the presence of free lipids (released from ruptured adipocytes) and peripheral blood elements (released from blood vessels severed during tissue harvest), and then disaggregated to free intact adipocytes and other cell populations from the connective tissue matrix. In some embodiments, the adipose-derived cells, e.g., adipose-derived cells comprising stem cells, adipose-derived cells comprising regenerative cells, adipose-derived cells comprising stem and other regenerative cells, and the like, are provided with blood vessel endothelial cells (BECs), BEC progenitors (EPCs), and adipose tissue-derived stem cells, adipose tissue-derived stromal cells, and other cellular elements, including adipose-derived microparticles. In some embodiments the adipose-derived cells, e.g., adipose-derived cells comprising stem cells, adipose-derived cells comprising regenerative cells, adipose-derived cells comprising stem and other regenerative cells, and the like, comprise cells that are in the form of aggregates or partially disaggregated fragments, for example, two or more vascular cells linked by extracellular matrix. In some embodiments such aggregates comprise large aggregates or fragments comprising more than 10 cells or more than 100 cells linked by extracellular matrix. Such aggregates may include, but are not limited to adipose-derived microparticles, blood or lymph vessel fragments in which several cells remain linked in an approximation of their original orientation to one another (including, by way of non-limiting example, vascular endothelial cells and pericytes or smooth muscle cells linked by some or all of the extracellular matrix that bound them together in the tissue prior to processing). In a particular embodiment such aggregates may comprise several hundred cells in contact or associated with fewer adipocytes than they were in the tissue prior to processing.

Rinsing is an optional but preferred step, wherein the tissue is mixed with a solution to wash away free lipid and single cell components, (and, optionally some adipose-derived microparticles) such as those components in blood, leaving behind intact adipose tissue fragments. In one embodiment, the adipose tissue that is removed from the patient is mixed with isotonic saline or other physiologic solution(s), e.g., Plasmalyte® of Baxter Inc. or Normosol® of Abbott Labs. Intact adipose tissue fragments can be separated from the free lipid and cells, which can include some adipose-derived microparticles, by any means known to persons of ordinary skill in the art including, but not limited to, filtration, decantation, sedimentation, or centrifugation. In some embodiments, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, and/or centrifugation techniques to separate the materials.

The intact tissue fragments are then disaggregated using any conventional techniques or methods, including mechanical force (mincing or shear forces), ultrasonic or other physical energy, lasers, microwaves, enzymatic digestion with single or combinatorial proteolytic enzymes, such as collagenase, trypsin, lipase, liberase H1, nucleases, or members of the Blendzyme family as disclosed in U.S. Pat. No. 5,952,215, “Enzyme composition for tissue dissociation,” expressly incorporated herein by reference in its entirety, and pepsin, or a combination of mechanical and enzymatic methods. For example, the cellular component of the intact tissue fragments may be disaggregated by methods using collagenase-mediated dissociation of adipose tissue, similar to the methods for collecting microvascular endothelial cells in adipose tissue, as disclosed in U.S. Pat. No. 5,372,945, expressly incorporated herein by reference in its entirety. Additional methods using collagenase that may be used are disclosed in, e.g., U.S. Pat. No. 5,830,741, “Composition for tissue dissociation containing collagenase I and II from clostridium histolyticum and a neutral protease” and by Williams, et al., 1995, “Collagenase lot selection and purification for adipose tissue digestion,” Cell Transplant 4(3):281-9, both expressly incorporated herein by reference in their entirety. Similarly, a neutral protease may be used instead of collagenase, as disclosed in Twentyman, et al. (Twentyman, et al., 1980, “Use of bacterial neutral protease for disaggregation of mouse tumours and multicellular tumor spheroids,” Cancer Lett. 9(3):225-8, expressly incorporated herein by reference in its entirety). Furthermore, the methods described herein may employ a combination of enzymes, such as a combination of collagenase and trypsin or a combination of an enzyme, such as trypsin, and mechanical dissociation.

Adipose tissue-derived cells, e.g., adipose-derived cells comprising stem cells, adipose-derived cells comprising regenerative cells, adipose-derived cells comprising stem and other regenerative cells, and the like, as well as some adipose-derived micro-particles (including, for example, apoptotic particles), may then be obtained from the disaggregated tissue fragments by reducing the number of mature adipocytes. In some embodiments, the adipose-derived micro-particles are associated with the adipose-derived cells (such as the adipose-derived regenerative cells). A suspension of the disaggregated adipose tissue and the liquid in which the adipose tissue was disaggregated is then passed to another container, such as a cell collection container. The suspension may flow through one or more conduits to the cell collection container by using a pump, such as a peristaltic pump, that withdraws the suspension from the tissue collection container and urges it to the cell collection container. Other embodiments may employ the use of gravity or a vacuum while maintaining a closed system. Separation of the cells (and associated adipose-derived microparticles) in the suspension may be achieved by buoyant density sedimentation, centrifugation, elutriation, filtration, differential adherence to and elution from solid phase moieties, antibody-mediated selection, differences in electrical charge, immunomagnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295, all incorporated herein by reference in their entirety. Many of these devices can be incorporated within the cell processing unit, while maintaining a closed system.

In some embodiments, the cells in the suspension are separated from the acellular component of the suspension using filter, such as a spinning membrane filter. In some embodiments, adipose-derived micro-particles can be separated from cellular components in the suspension, e.g., by passing the cell suspension though a filter that allows the micro-particles to pass through, while retaining cells. In some embodiments, the filter can have a pore size of about 1-5 μm (e.g., a 2 μm filter or the like). In some embodiments, adipose-derived micro-particles can be separated from the suspension using art-recognized techniques, such as ultra-centrifugation, e.g. about 20,000×g or higher, about 50,000×g or higher, about 100,000×g or higher, or the like, as described, for example, in György et al. (2011) Cell. Mol. Life Sci. 68:2667-2688, In some embodiments, the adipose-derived micro-particles described herein can be separated from the cellular component of the suspension using fluorescence activated cell sorting. Cell surface markers useful in separating and/or isolating the adipose-derived micro-particles disclosed herein include, for example, phosphatidylserine, CD34, CD44, CD105, CD106, CD166, 3G5, CD146, STRO-1, CD73, CD90, CD10, CD141, CD200, Mac-1, and the like. In some embodiments, the adipose-derived micro-particles can be separated and/or isolated from the cell suspension using dielectrophoresis.

In some embodiments, the cells in the suspension, which may or may not include adipose-derived microparticles, are separated from the acellular component using a centrifuge. In one such exemplary embodiment, the cell collection container may be a flexible bag that is structured to be placed in a centrifuge (e.g., manually or by robotics). In other embodiments, a flexible bag is not used. After centrifugation, the cellular component containing ADRCs forms a pellet, which may then be resuspended with a buffered solution so that the cells can be passed through one or more conduits to a mixing container, as discussed herein. The resuspension fluids may be provided by any suitable means. For example, a buffer may be injected into a port on the cell collection container, or the cell collection container may include a reserve of buffer that can be mixed with the pellet of cells by rupturing the reserve. When a spinning membrane filter is used, resuspension is optional since the cells remain in a volume of liquid after the separation procedure.

In one embodiment a subpopulation of the adipose-derived cells, e.g., adipose-derived cells comprising stem cells, adipose-derived cells comprising regenerative cells, adipose-derived cells comprising stem and other regenerative cells, and the like, is selected from other cells by short term adherence to a surface, for example, plastic. In some embodiments, the adipose-derived micro-particles are isolated from the cells or media from short-term adherence. In one embodiment the duration of adherence for the purpose of selection is approximately one hour. In a second embodiment the duration of adherence to the surface is 24 hours.

Although some embodiments described herein are directed to methods of fully disaggregating the adipose tissue to separate the active cells from the mature adipocytes and connective tissue, additional embodiments are directed to methods in which the adipose tissue is only partially disaggregated. For example, partial disaggregation may be performed with one or more enzymes, which are removed from at least a part of the adipose tissue early relative to an amount of time that the enzyme would otherwise be left thereon to fully disaggregate the tissue. Such a process may require less processing time and would generate fragments of tissue components within which multiple adipose-derived cells, e.g., adipose-derived cells comprising stem cells, adipose-derived cells comprising regenerative cells, adipose-derived cells comprising stem and other regenerative cells, and the like, remain in partial or full contact. In another embodiment mechanical force (for example ultrasound energy or shear force) is applied to prepare the cells, e.g., adipose-derived cells comprising stem cells, adipose-derived cells comprising regenerative cells, adipose-derived cells comprising stem and other regenerative cells, and the like, and/or fragments comprising adipose-derived cells isolated from all or some of the mature adipocytes with which they were associated in the tissue prior to processing.

In some embodiments, the tissue is washed with sterile buffered isotonic saline and incubated with collagenase at a collagenase concentration, a temperature, and for a period of time sufficient to provide adequate disaggregation. In a preferred embodiment, the collagenase enzyme used will be approved for human use by the relevant authority (e.g., the U.S. Food and Drug Administration). Suitable collagenase preparations include recombinant and non-recombinant collagenase. Non-recombinant collagenase may be obtained from F. Hoffmann-La Roche Ltd., Indianapolis, Ind. and/or Advance Biofactures Corp., Lynbrook, NY. Recombinant collagenase may also be obtained as disclosed in U.S. Pat. No. 6,475,764.

In one embodiment, solutions contain collagenase at concentrations of about 10 μg/ml to about 50 μg/ml (e.g., 10 μg/ml, 20 μg/ml, 30 μg/ml, 40 μg/ml, or 50 μg/ml) and are incubated at from about 30° C. to about 38° C. for from about 20 minutes to about 60 minutes. These parameters will vary according to the source of the collagenase enzyme, optimized by empirical studies, in order to confirm that the system is effective at extracting the desired cell populations in an appropriate time frame. A particular preferred concentration, time and temperature is 20 μg/ml collagenase (mixed with the neutral protease dispase; Blendzyme 1, Roche) and incubated for 45 minutes at about 37° C. An alternative preferred embodiment applies 0.5 units/mL collagenase (mixed with the neutral protease thermolysin; Blendzyme 3). In a particularly preferred embodiment the collagenase enzyme used is material approved for human use by the relevant authority (e.g., the U.S. Food and Drug Administration). The collagenase used should be free of micro-organisms and contaminants, such as endotoxin.

Following disaggregation the active cell population can be washed/rinsed to remove additives and/or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously. In one embodiment, the adipose-derived cells, e.g., adipose-derived cells comprising stem cells, adipose-derived cells comprising regenerative cells, adipose-derived cells comprising stem and other regenerative cells, and the like, are concentrated and the collagenase removed by passing the cell population through a continuous flow spinning membrane system or the like, such as, for example, the system disclosed in U.S. Pat. Nos. 5,034,135 and 5,234,608, all incorporated herein by reference in their entirety.

In addition to the foregoing, there are many known post-wash methods that may be applied for further purifying the adipose-derived cell population that comprises stem cells, regenerative cells, stem cells and regenerative cells, and the like. These include both positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

Many other conformations of the staged mechanisms used for cell processing will be apparent to one skilled in the art. For example, mixing of tissue and saline during washing and disaggregation can occur by agitation or by fluid recirculation. Cell washing may be mediated by a continuous flow mechanism such as the spinning membrane approach, differential adherence, differential centrifugation (including, but not limited to differential sedimentation, velocity, or gradient separation), or by a combination of means. Similarly, additional components allow further manipulation of cells, including addition of growth factors or other biological response modifiers, and mixing of cells with natural or synthetic components intended for implant with the cells into the recipient.

Post-processing manipulation may also include cell culture or further cell purification (Kriehuber, et al., 2001; Garrafa, et al., 2006). In some embodiments, once the adipose-derived cell population, cells, e.g., adipose-derived cells comprising stem cells, adipose-derived cells comprising regenerative cells, adipose-derived cells comprising stem and other regenerative cells, and the like, is obtained, it is further refined, concentrated, enriched, isolated, or purified using a cell sorting device and/or gradient sedimentation. Mechanisms for performing these functions may be integrated within the described devices or may be incorporated in separate devices. In many embodiments, however, a therapeutically effective amount of a concentrated population of adipose derived cells, e.g., adipose-derived cells comprising stem cells, adipose-derived cells comprising regenerative cells, adipose-derived cells comprising stem and other regenerative cells, and the like, is used to prepare a medicament for the reduction of inflammation (e.g., pancreatitis), wherein said concentrated population of cells is to be administered to a patient in need thereof without culturing the cells before administering them to the patient. That is, some embodiments concern methods to reduce pain, fibrosis, or both, wherein a therapeutically effective amount of a concentrated population of adipose derived cells, e.g., adipose-derived cells comprising stem cells, adipose-derived cells comprising regenerative cells, adipose-derived cells comprising stem and other regenerative cells, and the like, is administered to a patient in need thereof without culturing the cells before administering them to the patient.

In a preferred embodiment, the tissue removal system and processing set would be present in the vicinity of the patient receiving the treatment, such as the operating room or out-patient procedure room (effectively at the patient's bedside). This allows rapid, efficient tissue harvest and processing, and decreases the opportunity for specimen handling/labeling error, thereby allowing for performance of the entire process in the course of a single surgical procedure.

As described in U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004, one or more additives may be added to the cells during and/or after processing. Some examples of additives include agents that optimize washing and disaggregation, additives that enhance the viability of the active cell population (e.g., adipose-derived cells comprising stem cells, adipose-derived cells comprising regenerative cells, adipose-derived cells comprising stem and other regenerative cells, and the like), during processing, anti-microbial agents (e.g., antibiotics), additives that lyse adipocytes and/or red blood cells, or additives that enrich for cell populations of interest (by differential adherence to solid phase moieties or to otherwise promote the substantial reduction or enrichment of cell populations).

The adipose-derived cells, e.g., adipose-derived cells comprising stem cells, adipose-derived cells comprising regenerative cells, adipose-derived cells comprising stem and other regenerative cells, and the like, obtained as described herein can be cultured according to approaches known in the art, and the cultured cells can be used in several of the embodied methods. For example, adipose-derived cells, e.g., adipose-derived cells comprising stem cells, adipose-derived cells comprising regenerative cells, adipose-derived cells comprising stem and other regenerative cells, and the like, can be cultured on collagen-coated dishes or 3D collagen gel cultures in endothelial cell basal medium in the presence of low or high fetal bovine serum or similar product, as described in Ng, et al., November 2004, “Interstitial flow differentially stimulates blood and lymphatic endothelial cell morphogenesis in vitro,” Microvasc Res. 68(3):258-64, incorporated herein by reference. Alternatively, adipose-derived cells, e.g., adipose-derived cells comprising stem cells, adipose-derived cells comprising regenerative cells, adipose-derived cells comprising stem and other regenerative cells, and the like, can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture ADRCs, as well.

Examples of basal culture medium that can be used to culture adipose-derived cells, e.g., adipose-derived cells comprising stem cells, adipose-derived cells comprising regenerative cells, adipose-derived cells comprising stem and other regenerative cells, and the like, in vitro include, but are not limited to, EGM, RPMI, M199, MCDB131, DMEM, EMEM, McCoy's 5A, Iscove's medium, modified Iscove's medium or any other medium known in the art to support the growth of blood endothelial cells. Examples of supplemental factors or compounds that can be added to the basal culture medium that could be used to culture ADRCs include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyril cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, matrix metalloproteinases (such as MMP2 and MMP9), or any other compound known in the art to promote survival, proliferation or differentiation of endothelial cells.

Further processing of the cells may also include: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cell lines (including hybridoma cells); culture of cells for permeability studies; cells for RNAi and viral resistance studies; cells for knock-out and transgenic animal studies; affinity purification studies; structural biology applications; assay development and protein engineering applications.

In general, a system useful for isolating a population of adipose-derived cells, e.g., a population of adipose-derived cells comprising stem cells, a population of adipose-derived cells comprising regenerative cells, a population of adipose-derived cells comprising stem cells and regenerative cells, comprises a) a tissue collection container including i) a tissue collecting inlet port structured to receive adipose tissue removed from a subject, and ii) a filter disposed within the tissue collection container, which is configured to retain the adipose-derived cell population from said subject and to pass adipocytes, blood, and saline; b) a mixing container or cell processing chamber coupled to the tissue collection container by a conduit such that a closed pathway is maintained, wherein said mixing container receives said cell population and said mixing container comprises an additive port for introducing at least one additive to said population of adipose-derived cells; and an outlet port configured to allow removal of said population of adipose-derived cells from the mixing container or cell processing chamber for administration to a patient. In some embodiments, said mixing container or cell processing container further comprises a cell concentration device such as a spinning membrane filter and/or a centrifuge. Aspects of the embodiments disclosed herein also include a cell sorter, which is attached to said mixing chamber or cell processing chamber by a conduit and is configured to receive cells from said mixing chamber or cell processing chamber, while maintaining a closed pathway. Aspects of the embodiments above may also include a centrifuge attached to said mixing chamber or cell processing chamber by a conduit and configured to receive said population of adipose-derived cells, while maintaining a closed pathway, wherein said centrifuge comprises a gradient suitable for further separation and purification of said population of adipose-derived cells (e.g., ficoll-hypaque). Said centrifuge containing said gradient, which is configured to receive said population of adipose-derived cells may also be contained within said mixing container or cell processing chamber.

### Measuring ADRCs and ADRC Subsets in an Isolated Cell Population

A measurement, analysis, or characterization of the population of adipose-derived cells described herein to determine the presence of certain cells in the population can be undertaken within the closed system of a cell processing unit or outside of the closed system of a cell processing unit using any number of protein and/or RNA detection assays available in the art. Additionally, the measurement, analysis, or characterization of the adipose-derived cells, or certain cells (e.g., stem cells, progenitor cells, precursor cells, and the like), can be part of or can accompany the isolation procedure (e.g., cell sorting using an antibody specific for certain cell types (e.g., regenerative cells) or gradient separation using a media selective for certain cell types).

In some embodiments the measurement or characterization of the isolated cell population is conducted by detecting the presence or absence of a protein marker that is unique to certain cell types (e.g., adipose-derived regenerative cells, adipose-derived stem cells, adipose-derived precursor cells, adipose-derived progenitor cells, endothelial cells, endothelial precursor cells, or the like) is otherwise considered to confirm the presence of the specific cell type of interest by those of skill in the art. In addition to conventional Western blots using antibody probes specific for said proteins or markers, immunoselection techniques that exploit on cell surface marker expression can be performed using a number of methods known in the art and described in the literature. Such approaches can be performed using an antibody that is linked directly or indirectly to a solid substrate (e.g., magnetic beads) in conjunction with a manual, automated, or semi-automated device as described by Watts, et al., for separation of CD34-positive cells (Watts, et al., 2002, Variable product purity and functional capacity after CD34 selection: a direct comparison of the CliniMACS (v2.1) and Isolex 300i (v2.5) clinical scale devices,” Br J Haematol. 2002 July; 118(1):117-23), by panning, use of a Fluorescence Activated Cell Sorter (FACS), or other means.

Separation, measurement, and characterization can also be achieved by positive selection using antibodies that recognize cell surface markers or marker combinations that are expressed by certain cell types, but not by one or more of the other cell types or subpopulations present within the cell population. Separation, measurement, and characterization can also be achieved by negative selection, in which non-desired cell types are removed from the isolated population of adipose-derived cells using antibodies or antibody combinations that do not exhibit appreciable binding to ADRCs. Markers that are specifically expressed by ADRCs have been described. Examples of antibodies that could be used in negative selection include, but are not limited to, markers expressed by endothelial cells. There are many other antibodies well known in the art that could be applied to negative selection. The relative specificity of markers for ADRCs can also be exploited in a purification and/or characterization or measurement strategy. For example, a fluorescently-labeled ligand can be used in FACS-based sorting of cells, or an ligand conjugated directly or indirectly to a solid substrate can be used to separate in a manner analogous to the immunoselection approaches described above.

Measurement and characterization of the adipose-derived cell population to determine the presence or absence of specific cell types (e.g., specific types of regenerative cells) can also involve analysis of one or more RNAs that encode a protein that is unique to or otherwise considered by those of skill in the art to be a marker that indicates the presence or absence of a ADRCs. In some embodiments, for example, the isolated cell population or a portion thereof is analyzed for the presence or absence of an RNA that encodes one or more of, e.g., CD45, CD11b, CD14, CD68, CD90, CD73, CD31 and/or CD34. The detection of said RNAs can be accomplished by any techniques available to one of skill in the art, including but not limited to, Northern hybridization, PCR-based methodologies, transcription run-off assays, gene arrays, and gene chips.

### Compositions Comprising ADRCs and ADRC Subsets

In accordance with some of the aforementioned approaches, raw adipose tissue is processed to substantially remove mature adipocytes and connective tissue thereby obtaining a heterogeneous plurality of adipose tissue-derived cells comprising adipose-derived cells, e.g., adipose-derived cells comprising stem cells, adipose-derived cells comprising regenerative cells (e.g., one or more of stem cells, precursor cells, progenitor cells and the like), adipose-derived cells comprising stem and other regenerative cells, and the like, suitable for placement within the body of a subject. The extracted adipose-derived cells, e.g., adipose-derived cells comprising stem cells, adipose-derived cells comprising regenerative cells, adipose-derived cells comprising stem and other regenerative cells, and the like, may be provided in a neat composition comprising these cells substantially free from mature adipocytes and connective tissue or in combination with an inactive ingredient (e.g., a carrier) or a second active ingredient (e.g., adipose-derived stem cell and/or adipose-derived endothelial cell). The cells may be placed into the recipient alone or in combination (e.g., in a single composition or co-administered) with biological materials, such as cells, tissue, tissue fragments, or stimulators of cell growth and/or differentiation, supports, prosthetics, or medical devices. The composition may include additional components, such as cell differentiation factors, growth promoters, immunosuppressive agents, or medical devices, as discussed herein, for example. In some embodiments, the cells, with any of the above mentioned additives, are placed into the person from whom they were obtained (e.g., autologous transfer) in the context of a single operative procedure with the intention of providing a therapeutic benefit to the recipient.

Accordingly, aspects of the invention include compositions that comprise, consist, or consist essentially of a refined, enriched, concentrated, isolated, or purified adipose-derived cell population, e.g., adipose-derived cells comprising stem cells, adipose-derived cells comprising regenerative cells (e.g., one or more of stem cells, precursor cells, progenitor cells and the like), adipose-derived cells comprising stem and other regenerative cells, and the like, and mixtures of these cells with a biological material, additive, support, prosthetic, or medical device, including but not limited to, unprocessed adipose tissue, collagen matrix or support, cell differentiation factors, growth promoters, immunosuppressive agents, processed adipose tissue containing adipose-derived stem cells and/or progenitor cells, and cell populations already containing an enriched amount of ADRCs. In some embodiments, the aforementioned compositions comprise an amount or concentration of refined, isolated, or purified ADRCs that is greater than or equal to 0.5%-1%, 1-2%, 2%-4%, 4%-6%, 6%-8%, 8%-10%, 10%-20%, 20%-30%, 30%-40%, 40%-50%, 50%-60%, 60%-70%, 70%-80%, 80%-90%, or 90%-100% ADRCs, as compared to the total adipose-tissue cell population. In some embodiments, the ADRCs express an amount of, e.g., CD45, CD11b, CD14, CD68, CD90, CD73, CD31 and/or CD34.

In some embodiments, the adipose-derived cell e.g., adipose-derived cells comprising stem cells, adipose-derived cells comprising regenerative cells (e.g., one or more of stem cells, precursor cells, progenitor cells and the like), adipose-derived cells comprising stem and other regenerative cells, and the like, described herein is formulated in compositions that include at least one pharmaceutically acceptable diluent, adjuvant, or carrier substance, using any available pharmaceutical chemistry techniques. Generally, this entails preparing compositions that are essentially free of impurities that could be harmful to humans or animals.

Appropriate salts and buffers can be employed to stabilize and to facilitate uptake of the adipose-derived cells disclosed herein, e.g., the adipose-derived cell population that comprises ADRCs. Compositions contemplated herein can comprise an effective amount of the adipose-derived cells, e.g., adipose-derived regenerative cells stem cells, adipose-derived cells comprising regenerative cells, adipose-derived cells comprising stem and other regenerative cells and the like, and/or adipose-derived microparticles in a pharmaceutically acceptable carrier or aqueous medium.

Administration of the compositions described herein can be via any common route so long as the target tissue is available via that route. Compositions administered according to the methods described herein may be introduced into the subject by, e.g., by intravenous, intraarterial, intralymphatic, subcutaneous, intradermal, intramuscular, intramammary, intraperitoneal, intrathecal, retrobulbar, intrapulmonary (e.g., term release); by oral, sublingual, nasal, anal, vaginal, or transdermal delivery, by spray or other direct application, or by surgical implantation at a particular site. In each of these methods of administration the compositions may or may not comprise a carrier or other material that has the property of increasing retention of the composition at the site of action or of facilitating the traffic of the composition to the site of action. The introduction may consist of a single dose or a plurality of doses over a period of time. Vehicles for cell therapy agents are known in the art and have been described in the literature. See, for example Remington's Pharmaceutical Sciences, 18th Ed. (1990, Mack Publ. Co, Easton Pa. 18042) pp 1435-1712, incorporated herein by reference. Sterile solutions are prepared by incorporating the adipose-derived cell population e.g., adipose-derived cells comprising stem cells, adipose-derived cells comprising regenerative cells, adipose-derived cells comprising stem and other regenerative cells, and the like, in the required amount in the appropriate buffer with or without one or more of the other components described herein.

Combination therapy with any two or more agents described herein also is contemplated as an aspect of the invention. Similarly, every combination of agents described herein, packaged together as a new kit, or formulated together as a single composition, is considered an aspect of the invention. Compositions for use according to aspects of the invention preferably include the adipose-derived cells (e.g., adipose-derived regenerative cells, such as concentrated populations of adipose-derived cells comprising stem cells) and the like, formulated with a pharmaceutically acceptable carrier. The adipose-derived cells can also be applied with additives to enhance, control, or otherwise direct the intended therapeutic effect. For example, in some embodiments, the adipose-derived cells (e.g., adipose-derived regenerative cells, such as concentrated populations of adipose-derived cells comprising stem cells) can be further purified by use of antibody-mediated positive and/or negative cell selection to enrich the cell population to increase efficacy, reduce morbidity, or to facilitate ease of the procedure. Similarly, the adipose-derived cells disclosed herein can be applied with a biocompatible matrix, which facilitates in vivo tissue engineering by supporting and/or directing the fate of implanted cells, or the like. In the same way, cells can be administered following genetic manipulation such that they express gene products that are believed to or are intended to promote the therapeutic response provided by the cells.

The adipose-derived cells (e.g., adipose-derived regenerative cells, such as concentrated populations of adipose-derived cells comprising stem cells) and the like, can be applied alone or in combination with other cells, tissue, tissue fragments, growth factors, biologically active or inert compounds, resorbable plastic scaffolds, or other additive intended to enhance the delivery, efficacy, tolerability, or function of the population. The adipose-derived cell population that comprises ADRCs can also be modified by insertion of DNA or by placement in cell culture in such a way as to change, enhance, or supplement the function of the cells for derivation of a structural or therapeutic purpose.

In more embodiments, the adipose-derived cell population e.g., adipose-derived cells comprising stem cells, adipose-derived cells comprising regenerative cells (e.g., one or more of adipose-derived stem cells, precursor cells, progenitor cells, endothelial cells, or the like), are combined with a gene encoding a pro-drug converting enzyme which allows cells to activate pro-drugs within the site of engraftment, that is, within a tumor. Addition of the gene (or combination of genes) can be by any technology known in the art including but not limited to adenoviral transduction, “gene guns,” liposome-mediated transduction, and retrovirus or lentivirus-mediated transduction, plasmid, or adeno-associated virus. Cells can be implanted along with a carrier material bearing gene delivery vehicle capable of releasing and/or presenting genes to the cells over time such that transduction can continue or be initiated in situ. Particularly when the cells and/or tissue containing the cells are administered to a patient other than the patient from whom the cells and/or tissue were obtained, one or more immunosuppressive agents can be administered to the patient receiving the cells and/or tissue to reduce, and preferably prevent, rejection of the transplant.

Still more embodiments concern the ex vivo transfection of an adipose-derived cell population, e.g., adipose-derived cells comprising stem cells, adipose-derived cells comprising regenerative cells, adipose-derived cells comprising stem and other regenerative cells, and the like and subsequent transfer of these transfected cells to subjects. It is contemplated that such embodiments can be an effective approach to upregulate in vivo levels of the transferred gene and for providing relief from a disease or disorder resulting from under-expression of the gene(s) or otherwise responsive to upregulation of the gene (see e.g., Gelse, et al., 2003, “Articular cartilage repair by gene therapy using growth factor-producing mesenchymal cells,” Arthritis Rheum. 48:430-41; Huard, et al, 2002, “Muscle-derived cell-mediated ex vivo gene therapy for urological dysfunction,” Gene Ther. 9:1617-26; Kim, et al., 2002, “Ex vivo gene delivery of IL-1Ra and soluble TNF receptor confers a distal synergistic therapeutic effect in antigen-induced arthritis,” Mol. Ther. 6:591-600, all incorporated herein by reference). Delivery of an adipose-derived cell population, e.g., adipose-derived cells comprising stem cells, adipose-derived cells comprising regenerative cells, adipose-derived cells comprising stem and other regenerative cells and the like to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, 1998, “Human Gene Therapy,” Nature Suppl. to vol. 392 (6679):25-20, incorporated by reference herein. Gene therapy technologies are also reviewed by Friedmann, 1989, “Progress toward human gene therapy,” Science 244(4910):1275-1281, Verma (1990), “Gene therapy.” Scientific American 263(5): 68-84, and Miller (1992), “Human gene therapy comes of age,” Nature, 357:455-460, all incorporated by reference herein. An adipose-derived cell population, e.g., adipose-derived cells comprising stem cells, adipose-derived cells comprising regenerative cells, adipose-derived cells comprising stem and other regenerative cells, and the like, can be cultured ex vivo in the presence of an additive (e.g., a compound that induces differentiation or pancreatic cell formation) in order to proliferate or to produce a desired effect on or activity in such cells. Treated cells can then be introduced to a subject.

In some embodiments, the ex vivo gene therapy is conducted locally, e.g., to the site of a glial or fibrotic scar. For example, by using catheter-mediated transfer an adipose-derived cell population, e.g., adipose-derived cells comprising stem cells, adipose-derived cells comprising regenerative cells, adipose-derived cells comprising stem and other regenerative cells, and the like, can be transferred into a mammalian subject. Materials and methods for local delivery are reviewed, e.g., in Lincoff, et al. (1994), “Local drug delivery for the prevention of restenosis. Fact, fancy, and future,” Circulation, 90: 2070-2084, hereby expressly incorporated by reference. For example, adipose-derived cells e.g., adipose-derived cells comprising stem cells, adipose-derived cells comprising regenerative cells, adipose-derived cells comprising stem and other regenerative cells, and the like, can be provided to a subject by an infusion-perfusion balloon catheter (preferably a microporous balloon catheter), such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; Wolinsky, et al. (1990) (Wolinsky Infusion Catheter), “Use of a perforated balloon catheter to deliver concentrated heparin into the wall of the normal canine artery,” J. Am. Coll. Cardiol. 15: 475-481; and Lambert et al., 1993, “Local drug delivery catheters: functional comparison of porous and microporous designs,” Coron. Artery Dis. 4: 469-475. Use of such catheters for site-directed somatic cell gene therapy is described, e.g., in Mazur, et al., 1994, “Coronary restenosis and gene therapy,” Texas Heart Institute Journal 21: 104-111.

Aspects of the invention also concern the ex vivo transfection of adipose-derived cells, e.g., ADRCs (stem cells, progenitor cells, precursor cells, or combinations of stem cells and progenitor cells and/or precursor cells) with a gene encoding a therapeutic polypeptide, and administration of the transfected cells to the mammalian subject. Procedures for seeding a vascular graft with genetically modified endothelial cells are described in, e.g., U.S. Pat. No. 5,785,965, “VEGF gene transfer into endothelial cells for vascular prosthesis.”

### Administration of Regenerative Cells and Adipose-Derived Microparticles

The skilled person will readily appreciate that the regenerative cells, e.g., adipose-derived cells, (e.g., concentrated populations of adipose-derived cells comprising stem cells) and/or adipose-derived microparticles can be administered via a variety of different administration routes in accordance with the embodiments disclosed herein. For example, the regenerative cells described herein can be administered intravenously, intra-arterially, intramuscularly, intraperitoneally, intraocularly, parenterally, intrathecally, subcutaneously, into the lymphatic system (e.g., into a lymph vessel or lymph node) or transplanted directly into or onto the CNS, i.e., the brain or spinal cord.

In some embodiments the regenerative cells, e.g., adipose-derived cells, (e.g., concentrated populations of adipose-derived cells comprising stem cells), and/or adipose-derived microparticles are administered intra-arterially. Intraarterial administration of cells to the CNS has been demonstrated to be an effective, minimally invasive method for cell delivery to the CNS. See, e.g., Lu et al (2013), PLoS ONE 8(2): e54963, Guo et al. (2013) Stem Cell Research & Therapy 4:116; Osani, et al. (2012) Neurosurg. 70(2): 435-444; Misra, et al. (2012), Stem Cells Devel. 21(7): 1007-1015.

In some embodiments, the regenerative cells, e.g., adipose-derived cells, (e.g., concentrated populations of adipose-derived cells comprising stem cells) and/or adipose-derived microparticles can be delivered to the CNS intranasally, e.g., using the methods described in U.S. Patent Application Publication No. 2013/0028874.

In some embodiments, the regenerative cells, e.g., adipose-derived cells, (e.g., concentrated populations of adipose-derived cells comprising stem cells) and/or adipose-derived microparticles can be delivered to the CNS intravenously. Intravenous administration of cells to the CNS has been described, e.g., in Guzman et al. (2008) J. Neurosurg. 24(3-4):E15, and references cited therein.

The term or phrase “transplantation,” “cell replacement,” or “grafting” are used interchangeably herein and refer to the introduction of the cells described herein to target tissue. In some embodiments, the regenerative cells, e.g., adipose-derived cells, (e.g., concentrated populations of adipose-derived cells comprising stem cells) and/or adipose-derived microparticles can be grafted into the CNS or into the ventricular cavities or subdurally on to the surface of the subject's brain.

Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation.

Intraparenchymal transplantation can be effected using two approaches: (i) injection of regenerative cells, e.g., adipose-derived cells, (e.g., concentrated populations of adipose-derived cells comprising stem cells) into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor regenerative cells or by growing the regenerative cells, e.g., adipose-derived cells, (e.g., concentrated populations of adipose-derived cells comprising stem cells) in a substrate such as 3% collagen to form a plug of solid tissue which may then be implanted into the ventricle to prevent dislocation of the graft. For subdural grafting, the regenerative cells, e.g., adipose-derived cells, (e.g., concentrated populations of adipose-derived cells comprising stem cells) and/or adipose-derived microparticles may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The regenerative cells, e.g., adipose-derived cells, (e.g., concentrated populations of adipose-derived cells comprising stem cells) may also be introduced into the putamen, nucleus basalis, hippocampus cortex, striatum, substantia nigra or caudate regions of the brain, as well as the spinal cord.

The regenerative cells, e.g., adipose-derived cells, (e.g., concentrated populations of adipose-derived cells comprising stem cells) and/or adipose-derived microparticles may also be transplanted to a healthy region of the tissue. In some cases the exact location of the damaged tissue area may be unknown and the adipose-derived cells may be inadvertently transplanted to a healthy region. In other cases, it may be preferable to administer the adipose-derived cells to a healthy region, thereby avoiding any further damage to that region. Whatever the case, following transplantation, the cells and/or microparticles preferably migrate to or home to the damaged area.

For transplanting, a suspension comprising the regenerative cells, e.g., adipose-derived cells, (e.g., concentrated populations of adipose-derived cells comprising stem cells) and/or adipose-derived microparticles can be drawn up into the syringe and administered to anesthetized transplantation recipients. Multiple injections may be made using this procedure.

The suspension procedure thus permits grafting of the regenerative cells, e.g., adipose-derived cells, (e.g., concentrated populations of adipose-derived cells comprising stem cells) to any predetermined site in the brain or spinal cord, is relatively non-traumatic, allows multiple grafting simultaneously in several different sites or the same site using the same suspension, and permits mixtures of cells from different anatomical regions. Multiple grafts may consist of a mixture of cell types, and/or a mixture of transgenes inserted into the cells. Preferably from approximately 10⁴ to approximately 10⁸ cells are introduced per graft.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of the regenerative cells, e.g., adipose-derived cells, (e.g., concentrated populations of adipose-derived cells comprising stem cells) or solid tissue implants. Preferably, the site of implantation is dictated by the CNS disorder being treated.

### Combination Therapy

As explained in further detail below, some embodiments provide for treatment of subjects with combination therapy, i.e., one or more additional pharmaceutical agent, biologic agent, or other therapeutic agent, in addition to the regenerative cells, e.g., adipose-derived cells, (e.g., concentrated populations of adipose-derived cells comprising stem cells) described herein.

In some embodiments, the one or more additional “agents” described above can be administered in a single composition with the regenerative cells, e.g., adipose-derived cells, (e.g., concentrated populations of adipose-derived cells comprising stem cells) and/or adipose-derived microparticles. In some embodiments, the one or more additional “agents” can be administered separately from the regenerative cells, e.g., adipose-derived cells, (e.g., concentrated populations of adipose-derived cells comprising stem cells). For example, in some embodiments, one or more additional agents can be administered just prior to, or just after, administration of the regenerative cells, e.g., adipose-derived cells, (e.g., concentrated populations of adipose-derived cells comprising stem cells). As used herein, the term “just prior” can refer to within 15 minutes, 30 minutes, an hour, 2 hours, 3 hours, 4 hours, 5 hours, or the like. Likewise, the phrase “just after administration” can refer to within 15 minutes, 30 minutes, an hour, 2 hours, 3 hours, 4 hours, 5 hours, or the like.

Additional agents useful in combination therapy in the methods described herein include, for example, statins, growth factors, cytokines, platelet rich plasma, as well as other agents known in the art to have beneficial effects in central nervous system diseases or disorders.

a. Statins

Statins have been shown to have beneficial effects, such a neuroprotective and anti-inflammatory effects, in CNS disorders such as multiple sclerosis, traumatic brain injury, and the like. See, e.g., Xiong, et al., Curr. Opin. Investig. Drugs, 11(3): 298-308; Chen, et al. (2003) Ann. Neurol. 53(6): 743-751; Chen et al. (2007) Life Sci. 81(4): 288-298, Lu, et al. (2004) J. Neurosurg. 101(5): 813-821; Li, et al. (2009) Neurosurg. 65(1): 179-186. Non-limiting examples of statins useful in the embodiments disclosed herein include, for example, atorvastatin, lorvastatin, simvastatinfluvastatin, lovastatin, pravastatin, rosuvastatin, and the like.

Accordingly, some embodiments provide treatment of CNS disorders as disclosed herein wherein the subject is administered a therapeutically effective amount of one or more statins, in addition to the regenerative cells, e.g., adipose-derived cells, (e.g., concentrated populations of adipose-derived cells comprising stem cells) and/or adipose-derived microparticles. In some embodiments, the combination of a statin with the regenerative cells, e.g., adipose-derived cells, (e.g., concentrated populations of adipose-derived cells comprising stem cells) disclosed herein provides a more than additive beneficial effect, i.e., the combination therapy provides synergistic benefits. Accordingly, in some embodiments, one or more statins are administered concomitantly with, prior to, or following the administration of the regenerative cells, e.g., adipose-derived cells, (e.g., concentrated populations of adipose-derived cells comprising stem cells).

b. Growth Factors

Several growth factors have been shown to have beneficial effects, e.g., neruoportection, angiogenesis, improvement in functional recorder, and the like, in CNS disorders. See, e.g., Thau-Zuchman et al. (2012) J. Mol. Neurosci. 47(1): 166-172; Thau-Zuchman, et al. (2006) J. Neurosurg. 105(6): 843-852.

In some embodiments, subjects can be administered one or more growth factors. For example, in some embodiments, growth factors are administered concomitantly with, prior to, or following the administration of the regenerative cells, e.g., adipose-derived cells, (e.g., concentrated populations of adipose-derived cells comprising stem cells) and/or adipose-derived microparticles.

Non-limiting examples of growth factors useful in the embodiments disclosed herein include, but are not limited to, angiogenin, angiopoietin-1 (Ang-1), angiopoietin-2 (Ang-2), brain-derived neurotrophic factor (BDNF), Cardiotrophin-1 (CT-1), ciliary neurotrophic factor (CNTF), Del-1, acidic fibroblast growth factor (aFGF), basic fibroblast growth factor (bFGF), follistatin, ganulocyte colony-stimulating factor (G-CSF), glial cell line-derived neurotrophic factor (GDNF), hepatocyte growth factor (HGF), scatter factor (SF), Interleukin-8 (IL-8), leptin, midkine, nerve growth factor (NGF), neurotrophin-3 (NT-3), Neurotrophin-4/5, Neurturin (NTN), placental growth factor, Platelet-derived endothelial cell growth factor (PD-ECGF), Platelet-derived growth factor-BB (PDGF-BB), Pleiotrophin (PTN), Progranulin, Proliferin, PBSF/SDF-1, Transforming growth factor-alpha (TGF-alpha), Transforming growth factor-beta (TGF-beta), Tumor necrosis factor-alpha (TNF-alpha), Vascular endothelial growth factor (VEGF), vascular permeability factor (VPF), and the like.

c. Steroids

Steroids such as progesterone have been shown to have beneficial effects in CNS disorders. See, e.g., Djebaili, et al. (2005) J. Neurotrauma 22(1): 106-118. Accordingly, in some embodiments, subjects are administered on or more steroids, in addition to the regenerative cells, e.g., adipose-derived cells, (e.g., concentrated populations of adipose-derived cells comprising stem cells) and/or adipose-derived microparticles disclosed herein. For example, in some embodiments, steroids are administered concomitantly with, prior to, or following the administration of the regenerative cells, e.g., adipose-derived cells, (e.g., concentrated populations of adipose-derived cells comprising stem cells).

Non-limiting examples of steroids useful in the embodiments disclosed herein include, but are not limited to, progestegens, e.g., progesterone, and the like; corticosteroids, e.g., prednisone, aldosterone, cortisol, and the like, androgens, e.g., testosterone, and the like, and estrogens.

d. Platelet Rich Plasma (“PRP”)

Platelet rich plasma (“PRP”) has been demonstrated to promote remyelinization of peripheral nerves. See, e.g., Shen, et al. (2009) Med. Hypoth. 73(6):1038-40. PRP has been described for the treatment of neurodegenerative and psychiatric disorders. See, e.g., WO 09/155069. Accordingly, in some embodiments, subjects are administered platelet rich plasma, in addition to the regenerative cells, e.g., adipose-derived cells, (e.g., concentrated populations of adipose-derived cells comprising stem cells) and/or adipose-derived microparticles disclosed herein. For example, in some embodiments, steroids are administered concomitantly with, prior to, or following the administration of the adipose-derived cells.

The term “PRP” as used herein refers to a concentration of platelets greater than the peripheral blood concentration suspended in a solution of plasma. Methods for isolating PRP useful in the embodiments disclosed herein are known in the art. See, e.g., WO 09/155069. Platelets or PRP can suspended in an excipient other than plasma or the platelet composition includes other excipients suitable for administration to a human or non-human animal including, but not limited to isotonic sodium chloride solution, physiological saline, normal saline, dextrose 5% in water, dextrose 30% in water, Ringer solution, lactated Ringer solution, Ringer lactate, Ringer lactate solution, and the like. Typically, platelet counts in PRP as defined herein range from 500,000 to 1,200,000 per cubic millimeter, or even more. PRP may be obtained using autologous, allogenic, or pooled sources of platelets and/or plasma. PRP may be obtained from a variety of animal sources, including human sources. In preferred embodiments, PRP according to the invention is buffered to physiological pH.

e. Selective Serotonin Reuptake Inhibitors

Selective serotonin reuptake inhibitors (SSRIs) are the only FDA-approved pharmaceuticals used in the treatment of PTSD.

In some embodiments, in addition to the administration of the regenerative cells, e.g., adipose-derived cells, (e.g., concentrated populations of adipose-derived cells comprising stem cells) according to the embodiments described herein, subjects can be administered one or more SSRIs. Non-limiting examples of SSRIs useful in the embodiments disclosed herein include, but are not limited to Celexa, Lexapro, Luvox, Paxil, Prozac, Zoloft, and the like. The skilled artisan will readily appreciate that dosages and administration of SSRIs are widely known in the art.

### EXAMPLES

### Example 1

### Treatment of Traumatic Brain Injury with Adipose-Derived Cells

A blunt TBI is delivered to rats using by a captured-bolt mechanism (MyNeuroLab.com—Blackmark, Stereotaxic Impactor). Each animal is placed in a directing framework with the device set to deliver the appropriate energy to an area on the skull 8 mm behind the bregma, 5 mm lateral to the midline. After the blunt trauma is conveyed to the rat brain, the device is removed from the frame. A delivery methodology is placed into each animal, fixed in place and imaging is done with specialized CT Scans and/or MRI of the brain. Approximately 10⁴-10⁸ adipose-derived cells are administered to the treated animals.

Functional Testing for Rats Following Rota rod test: A Rota rod apparatus (Columbus Instruments, Columbus, Ohio) is used to measure motor coordination and balance of the animal. Prior to procedures/operations, rats are trained on the Rota rod at a constant speed (16 rpm) until the rat is able to remain on the machine for a minimum of 60 seconds. Following blunt TBI and adipose-derived cell treatment, rats are tested on the Rota rod performance on Day 3, 7, 14, 21, and 28 (or longer if necessary). On testing days, each rat receives two trials at each of the two constant speed levels (i.e. 20 and 25 rpm) and two trials of an accelerating speed (4-40 rpm within 2 min). The latency to fall off the Rota rod for each trial and the time on the Rota Rod is recorded and used in subsequent analysis.

Novel Object Recognition (NOR) Test: Rats have an intrinsic nature to explore a novel environment. The NOR task utilizes the rat's “curiosity” to measure its ability to discriminate an “old” familiar object from a novel object based on its memory of the “old” object (Ennaceur, A. and Delacour, J., Behav Brain Res. 1988 Nov. 1; 31(1):47-59). On Day 7, 14, 21, and 28, rats are placed in a plastic circular container (20 inches diameter.times.17 inches high) which contains two identical objects (12 inch apart) and allowed to explore for 5 min and then returned to their home cage. Three (3) hours later, one of the objects is replaced by a novel object distinctively different from the other object and the rat is placed in the container again for 5 min to explore both objects. The exploration time of the animal in both sessions is recorded by a video camera for analysis. A discrimination index (DI) is calculated as: [(Time spent exploring new object)−(Time spent exploring old object)]/[(Time spent exploring new object)+(Time spent exploring old object)]. The higher DI gives a better indication that the rat discriminates two objects based on memory of the old object.

Treated animals exhibit improved motor and cognition compared to controls.

### Example 2

### Reducing Vulnerability to Second Injury

An at-risk subject is identified as having suffered a traumatic brain injury. Within 24 hours following injury, cerebral glucose metabolism (CMRgluc) is measured in the subject using [18F]-2fluoro-2-deoxy-D-glusoe positron emission tomography (FDG-PET). The subject's cerebral glucose metabolism is below 10 μmol/100 g/min.

Within 24 hours following injury, the subject is administered a composition comprising adipose-derived cells (e.g., adipose-derived regenerative cells, such as concentrated populations of adipose-derived cells comprising stem cells) as described herein above inter-arterially and advised not to resume any at-risk activity for 24 hours. The subject's CMRglug increases above 15 μmol/100 g/min within 24 hours following treatment, thereby indicating that that the subject is not at risk of worsened outcome in the event of a subsequent insult (e.g., TBI, ischemic insult, or the like).

### Example 3

### Treatment of Amyotrophic Lateral Sclerosis

In this example, an established animal model for amyotropyhic lateral sclerosis is used to demonstrate efficacy of the adipose-derived cells disclosed herein for treatment of ALS. Transgenic mice carrying high copy numbers of the transgene with the G93A human SOD1 mutation are used in this study which is a modification of the study described by Feng et al., (2008) Neuroscience 155:567-572. All transgenic mice are genotyped by PCR amplification of DNA extracted from the tails to identify the SOD1 mutation.

Mice are divided into vehicle and treatment groups. Treatment with adipose-derived cells as disclosed herein or sham vehicle is initiated 30 days after birth and continued until the end stage. Each animal is given a first dose followed by a subsequent weekly dose of adipose-derived cells via intravenous injection (approximately 1×10⁵ cells/injection). All animals are maintained on a 12 hours light/dark cycle. Behavior tests are performed during the light period. Various tests are routinely performed starting from 12 weeks of age until death.

Rotarod performance test: Motor coordination is assessed by measuring the length of time for which mice remained on the rotating rod (16 r.p.m.). Three trials are given to each animal and the longest retention time is used as a measure of competence at the task. The evaluation scores are: grade 0, >180 s; grade 1, 60-180 s; grade 2, <60 s; grade 3, falling off the rod before rotation.

Postural reflex test: This is conducted essentially as described by Bederson et al., (1986), 17:472-476 to examine the strength of the forelimbs. The deficits are scored as follows: grade 0, no evidence of paralysis; grade 1, forelimb flexion upon tail suspension; grade 2, decreased resistance to lateral push (and forelimb flexion) without circling; grade 3, same as grade 2 but with circling; grade 4, unable to walk but maintaining upright body position; grade 5, complete paralysis

Screen test: This test serves as an indicator of general muscle strength. The animal is placed on a horizontally positioned screen with grids. The screen is then rotated to the vertical position. The deficit scores are: grade 0, grasping the screen with forepaws for more than 5 s; grade 1, temporarily holding the screen without falling off; grade 2, same as grade 1 but falling off within 5 s; grade 3, falling off instantaneously.

Animals receiving treatment with adipose-derived cells exhibited improvement or stabilization one or more of the above scores, indicative of a therapeutic effect of adipose-derived regenerative element therapy.

### Example 4

### Treatment of Amyotrophic Lateral Sclerosis

This example describes the use of adipose-derived cells in the treatment of subjects diagnosed with ALS.

Subjects eligible for treatment meet the following criteria: Subjects diagnosed using the parameters of the World Federation of Neurology criteria; More than 6 and less than 36 months of evolution of the disease; Medullar onset of the disease; More than 20 and less than 65 years old; Forced Vital Capacity equal or superior to 50%; and Total time of oxygen saturation <90% inferior to 2% of the sleeping time. Subjects with one of the following criterion are excluded: neurological or psychiatric concomitant disease; concomitant systemic disease; treatment with corticosteroids, immunoglobulins or immunosuppressors during the last 12 months.

Primary outcome measures of the study include survival rate and functional rating scale (ALS-FRS) (Cederbaum, et al., (1999) J. Neurol. Sci. 169(1-2)13-21; available at the hypertext transfer protocol address: www.outcomes-umassmed.org/als/sf12.aspx), each assessed every 3 months. Secondary outcome measures include: MRC and Norris scales, Manual Muscle Test (MMT), and adverse events. Secondary outcome measures are each assessed every 3 months.

Adipose-derived cells as disclosed herein are isolated from the subject. A portion of the adipose-derived cells are cryopreserved for later use. Each subject is administered a dose of adipose-derived cells (approximately 1×10⁶ cells) via intraarterial injection every 3 months (2^{nd} and subsequent doses are obtained from the cryopreserved cells from the individual).

Subjects show improvement in one or more of the primary and secondary outcome measures.

### Example 5

### Treatment of Parkinson's Disease with Adipose-Derived Cells

In this example, an established animal model for Parkinson's disease is used to demonstrate efficacy of the adipose-derived cells disclosed herein for treatment. -Methyl-4-phenyl-1,2,3,6-tetrahydropyridine (MPTP) is known to cause an acute Parkinson's syndrome (parkinsonism) when administered to humans. The syndrome resembles spontaneous parkinsonism in terms of cardinal symptoms (muscular rigidity, bradycinesia, and resting tremor) and pathological phenomena (extensive degeneration of the nigrostriatal dopamine system) MPTP-treated mice also exhibit the syndrome similar to parkinsonism See, Heikkila, et al. (1984) Science, 224:1451. Accordingly, MPTP-treated C57BL/6 mice have been reported to serve as a suitable model for Parkinson's disease. In this strain of mice, striatal dopamine is remarkably decreased and locomotor activity is profoundly depressed. See, Sundström, et al. (1990) Brain Res., 528:181.

30 mg/kg MPTP (Aldrich Chemical Co., Inc.) is intraperitoneally administered to each mouse once a day for five consecutive days to “treatment” and “control” groups of 7-week-old male C57BL/6 mice. Thirty minutes after the final MPTP administration, adipose-derived cells as disclosed herein (approximately 1×10⁷ cells) or vehicle alone are administered intracranially to separate groups of mice. The amount of active movements (horizontal activity) of each mouse is measured by using Automex-II (Columbus Instruments International Corp.) for the period of 30 minutes starting 30 minutes after the administration of the adipose-derived cells. The average counts of the active movements of the adipose-derived cell treated group are compared with those of the control groups.

Mice receiving adipose-derived cell treatment show significant improvement in the number of active movements compared to the control group, demonstrating efficacy of the adipose-derived cells in treatment of Parkinson's disease.

Mice are subsequently sacrificed, and histological analysis of the brain is performed to test the effect of adipose-derived cell therapy on the glial response in treated animals, the complement receptor Mac-1 is used as a specific marker of microglia. In saline-injected mice, ramified resting microglia are faintly stained with the Mac-1 antibody in the substantia nigra (“SN”), and to an even lesser extent in the surrounding ventral midbrain (FIG. 4g). In MPTP injected mice, Mac-1 staining in the SN increases markedly after intoxication and is accompanied by typical morphological changes, such as cell body enlargement, shortening of processes, and loss of ramification. In mice receiving adipose-derived cell treatment, there is strong reduction of Mac-1 expression at day 2 post MPTP, indicating a reduction in microglial activation. MPTP also induces the expression of iNOS in the SN, revealing cells with cytoplasmic staining and morphology of activated microglia at day 2 post MPTP injections, which are absent in adipose-derived cell-treated animals.

### Example 6

### Efficacy of Adipose-Derived Cells in Treatment of Alzheimer's Disease

This example describes the use of adipose-derived cells as disclosed herein in a well-accepted animal model to test efficacy in treatment of Alzheimer's disease. In the experiments, 10 male Wistar rats of 7 weeks old were used for one experimental group. Rats are anesthetized and fixed in a brain stereotaxic apparatus. The skull of each rat is exposed, and small windows were made at two sites: 1.4 mm posterior to the bregma and 2.8 mm left and right of the midline. A catheter is inserted from the exposed dura mater into the basal ganglia located at 7.6 mm in a ventral direction, and ibotenic acid solution prepared by dissolving 5 μg ibotenic acid in 0.5 μL phosphate buffer (50 mM) was injected over 5 minutes to destroy the basal ganglia, thereby preparing Alzheimer's disease model rats. Rats of sham operation group receive injection of 0.5 μK, phosphate buffered saline (50 mM) instead of ibotenic acid solution. The basal ganglia are nuclei of origin of acetylcholine neurons which project to the cerebral cortex and play a critical role in learning and memory function, and it is known that aberrant functioning of acetylcholine neurons leads to learning and memory dysfunction in rats whose basal ganglia have been destroyed. Shinoda et al., (1999) Behav. Brain Res. 99:17.

Learning and memory function of the subject rats are evaluated by the Morris water maze test. Specifically, a round pool with a diameter of 150 cm, a height of 45 cm and a depth of 30 cm is provided, and a colorless, transparent platform with a diameter of 12 cm is arranged at about 1 cm below water surface. The water temperature of the round pool is set at 23±1° C. The illuminations of the room where the round pool was provided are indirect lighting, and visual cues (calendar, desk, personal computer) for the subject rats are arranged around the round pool. The arrangement of these visual cues is not changed at all during the test period. The swimming time until a subject rat placed at the arbitrary start position in the round pool reached the platform (hereinafter “Escape latency”) is measured by recording the movement locus of the subject rat with a video image behavioral analysis system.

The day when destruction of the basal ganglia (hereinafter “destruction treatment”) was performed is taken as Day 0, and on Day 9 after destruction treatment, subject rats are made to swim in the round pool in which the platform was not arranged so that they are acclimated to water.

Measurement of the Escape latency is carried out from Day 10 to Day 12 after destruction treatment, and three trials are performed each day with 30-minute intervals. The start position is changed every trial, but the platform is arranged at the same position through all the trials. Subject rats which do not reach the platform 90 seconds after the start are allowed to stay on the platform for 30 seconds after swimming. The mean value of the Escape latencies obtained from three trials is taken as an Escape latency of each subject rat.

Adipose-derived cells (“ADCs”) as disclosed herein (approximately 1×106 cells) are intra-arterially administered to subject rats 60 minutes after the destruction treatment. 0.5% MC is administered to the rats of sham operation group and vehicle-administered group, but not the ADC-treatment group.

In the sham operation group, Escape latency of the rats is shortened as a trial of Morris water maze was repeated, which indicates that the rats of sham operation group learned and memorized the position where the platform is arranged. On the other hand, in the vehicle-administered group, Escape latency of the rats is statistically significantly prolonged compared to the sham operation group, which clearly indicates that the learning and memory function was impaired in the rats of vehicle-administered group by destruction of the basal ganglia. In the ADC-administered group, Escape latency of the rats is statistically significantly shortened compared to the vehicle-administered group, and found to be the same level as of the sham operation group rats, indicating efficacy of ADC's in the treatment of Alzheimer's disease.

### Example 7

### Efficacy of Adipose-Derived Cells in Treatment of Multiple Sclerosis

This example describes the use of adipose-derived cells as disclosed herein in a well-accepted animal model to test efficacy in treatment of multiple sclerosis. Experimental autoimmune encephalomyelitis (EAE) is a generally accepted animal model of MS, and is used by researchers worldwide to study therapeutics potentially useful in treating MS as well as studying a model of MS.

EAE is induced in SJL mice by subcutaneous immunization with a peptide from proteolipid protein (i.e. PLPi₃₉-i₅i) in complete adjuvant. After 1 and 3 days, the mice are injected intravenously with 10⁹ heat-killed Bordetella pertussis bacteria to increase the permeability of the blood-brain barrier. EAE develops as follows:

1. Activation of T cells by macrophages and dendritic cells that present PLPi₃₉₋-ₗ₅ₗ

2. Elevated expression of interleukin-12 in macrophages and dendritic cells.

3. Differentiation of T cells into effector cells that secrete pro-inflammatory cells and express unique chemokine receptors

4. Increased permeability of the blood-brain-barrier

5. Migration of effector cells and monocytes into brain parenchyma against a gradient of chemokines

6. Local (re-)activation of inflammatory cells

7. Release of mediators of inflammation and destruction of oligodendrocytes and myelin.

Clinical symptoms develop starting approximately on day 11 after immunization. These symptoms include decrease in body weight and the development of paresis and paralysis. After recovery from the first relapse, several relapses and remissions may occur in about 65% of the animals. Eventually, the paralytic symptoms are chronic in nature.

### Experimental Procedures

Female SJL mice (Age: 9-12, weight: 16-20 grams; Harlan) are acclimatized for 13 days prior to the start of the study, housed under clean conventional conditions, and were randomized over the treatment groups. The mice were divided into three groups of 12 mice each: a) Saline (day 0 to day 5); b) Treatment group 1×10⁵ adipose-derived cells as described herein, delivered intra-arterially; and c)

To induce EAE, all mice receive subcutaneous injections of 75 μg PLPi₃₉-i₅i (Isogen Bioscience B.V.) in a 200 μl emulsion (1:1) of phosphate-buffered saline and complete H37 Ra adjuvant distributed over four sites in the flanks of the mice. The mice also receive intravenous injections of 10⁹ Bordetella pertussis bacteria on days 1 and 3. All mice were monitored for a total of 42 days. Daily measurements of body weight and disability score are taken to evaluate the clinical signs of EAE. Animals are considered to be affected by EAE when a cumulative score of at least 3 is reached within a period of three consecutive days. The maximum weight loss, maximum EAE and cumulative EAE score is calculated for each mouse. In addition to the total monitoring period, the maximum and cumulative EAE scores are separately determined for the first and second phases of EAE (defined as days 0-20 and days 21-42 respectively) for the mice. In addition, the mean EAE score is determined for the early second phase of EAE (days 21-31) and the late second phase of EAE (days 32-42), which late second phase approximates RRMS phase of MS.

The following scoring system is used to monitor the degree of disability in the EAE model (Kono et al., (1988) J Exp Med 168, 213-227):

Disability scoring system to determine the severity of EAE

0: no disease

0.5: tail paresis or partial paralysis

1 complete tail paralysis

2 paraparesis: limb weakness and tail paralysis

2.5: partial limb paralysis

3 complete hind- or front limb paralysis

3.5: paraplegia

4 quadriplegia, moribund

5 death due to EAE

All vehicle treated mice develop EAE. Mice treated with adipose-derived cells exhibit significant decrease in the mean EAE score compared to the vehicle control group, indicating that the adipose-derived cells decrease the severity of symptoms of EAE, and are useful for the prophylaxis and/or treatment of multiple sclerosis.

### Example 8

### Adipose-Derived Cells Treat PTSD in Experimental Animal Models

Several animal models specific for PTSD useful for testing anxiolytic efficacy and efficacy of treatment of PTSD have been established. Exemplary animal models useful in the embodiments disclosed herein include, for example, exposure to inescapable electric shock (see, e.g., Maier et al. (2001) Biol Psychiatry. 2001; 49:763-73); high and low anxiety behavior rats (see, e.g., Muigg et al. (2008) Eur. J. Neurosci. 28:2299-2309); single prolonged stress rats (see, e.g., Liberzon (1999) Neurochem. 11(1):11-17, and the predator exposure model (Zoladz et al., (2008) Stress 11, 259-281).

This example describes the use of adipose-derived cells as disclosed herein in a well-accepted animal model (enhanced single prolonged stress procedure, i.e., ESPS) to test efficacy of the adipose-derived elements disclosed herein in the treatment of PTSD.

Following a period of acclimatization, rats are randomly assigned to one of four groups (1) Control (no exposure to ESPS, but administered vehicle); (2) Vehicle (exposure to ESPS, and administered vehicle); and (3) Treatment (exposure to ESPS and treatment with adipose-derived cells after ESPS).

Behavior testing is done at a fixed time during testing days and animals are habituated in the testing room 15 min before behavioral testing.

For ESPS, rats are restrained for 2 hours, immediately followed by forced swimming for 20 min in 24° C. water contained in a clear cylinder. After 15 min of recuperation, animals are exposed to diethyl ether until the lose consciousness, and then moved into a shock chamber. After a 30 minute recovery time, rats are administered a single electric foot shock (1 mA for 4 s) via metal grids installed in the bottom of the chamber.

14 days after ESPS, the rats are in groups (2) and (3) are administered vehicle or ADC's, respectively. All rats are subsequently tested in an open field test and an elevated plus-maze test.

To assess anxiety, rats are subjected to an open field test. Rats are placed in a black acrylic box placed in a soundproof box. The acrylic box is 47 cm³. Recording is performed in the soundproof box illuminated by a red fluorescent light. Anxiety in open spaces forces rats to spend most of their time next to the border of the arena. The fraction of time the rats spend exploring the center of the arena versus the edges is used to quantify anxiety and exploratory drive. Rats are recorded for 15 min.

Rats are also subjected to an elevated plus-maze test. A plexiglass apparatus that consists of a plus-shaped platform elevated 50 cm above the floor is used. Two of the opposing arms (50×10 cm) are enclosed by 40 cm high side and end walls (closed arms), and the other two arms are not installed with walls (open arms). rats are placed in the central area (10×10 cm) of the maze, facing a closed arm. The time spent in all areas is recorded.

Rats receiving treatment with adipose-derived cells exhibit improved outcome as compared to vehicle controls as assessed in the open field and elevated plus-maze tests, demonstrating the efficacy of adipose-derived cells in the treatment of PTSD.

### EQUIVALENTS

The compositions and methods disclosed herein are not to be limited in scope by the specific embodiments described herein. Indeed, various modifications of the compositions and methods in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method for reducing vulnerability to secondary brain insult in a subject in need thereof, comprising:
identifying a subject that has suffered one or more mild or severe traumatic brain injuries; and
administering a composition comprising regenerative cells to said subject.

2-26. (canceled)
